(19) United States
(12) Patent Application Publication
(10) Pub. No.: US 2025/0367292 A1
(43) Pub. Date: Dec. 4, 2025

(54) HLA SUPERAGONISTS AND USES THEREOF

(71) Applicant: UNIVERSITY HEALTH NETWORK, Toronto (CA)

(72) Inventors: Naoto HIRANO, Toronto (CA); Kenji MURATA, Toronto (CA); Dalam LY, Toronto (CA); Hiroshi SAIJO, Toronto (CA); Yukiko MATSUNAGA, Toronto (CA)

(73) Assignee: UNIVERSITY HEALTH NETWORK, Toronto, ON (CA)

(21) Appl. No.: 18/703,663

(22) PCT Filed: Oct. 21, 2022
(86) PCT No.: PCT/IB2022/060159

§ 371 Date: Apr. 22, 2024

Related U.S. Application Data
(60) Provisional application No. 63/262,941, filed on Oct. 22, 2021.

(51) Int. Cl. A61K 40/42; A61K 40/19; A61K 40/24; A61P 35/00; C07K 14/74

(52) U.S. Cl. CPC A61K 40/4213; A61K 40/19; A61K 40/24; A61P 35/00; C07K 14/70539; A61K 2239/57

(57) ABSTRACT

The present disclosure is directed to methods of modifying an HLA-binding pocket in an HLA molecule in a subject. Some aspects are directed to HLA molecules comprising a modified HLA-binding pocket, where the HLA molecule has increased affinity for a peptide, e.g., an antigen. Other aspects are directed to compositions comprising the same and methods of using the same.

### CROSS-REFERENCE TO RELATED APPLICATIONS

This application claims priority benefit of U.S. Provisional Application No. 63/262,941, filed Oct. 22, 2021, which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4285_022PC01_Seqlisting_ST26; Size: 2,448 bytes; and Date of Creation: Oct. 21, 2022) filed with the application is herein incorporated by reference in its entirety.

### FIELD OF THE DISCLOSURE

The present disclosure provides methods of enhancing an immune response to an antigen by modifying a human leukocyte antigen (HLA)-binding pocket expressed on an antigen-presenting cell.

### BACKGROUND OF THE DISCLOSURE

Immunotherapy has immerged as a critical tool in the battle against a variety of diseases, including cancer. T cell therapies are at the forefront of immunotherapeutic development, and adoptive transfer of antitumor T cells has been shown induce clinical responses in cancer patients. Though many T cell therapies target mutated tumor antigens, the vast majority of neoantigens are not shared and are unique to each patient.

The immunogenicity of a T cell antigen is a major factor driving effective cancer immunotherapy and is directly correlated with the ability of the antigenic epitope to bind HLA and be stably presented to T cells. This presents a limitation to the development of cancer immunotherapies, as many tumor-associated epitopes, which are derived from self-antigens, elicit relatively weak T cell responses. This has traditionally been overcome through the generation of “heteroclitic” peptides, a laborious, low-throughput approach based on trial and error, in which individual epitopes are modified to bind HLA molecules with enhanced interaction strength.

As such, there remains a need in the field of immunotherapy for novel means of improving the immunogenicity of potential target antigens.

### SUMMARY OF THE DISCLOSURE

Some aspects of the present disclosure are directed to methods of conditioning a subject in need of a therapy comprising modifying a human leukocyte antigen (HLA)-binding pocket of an HLA molecule expressed on a cell of the subject.

Some aspects of the present disclosure are directed to methods of enhancing an immune response in a subject in need thereof, comprising modifying an HLA-binding binding pocket of an HLA molecule expressed on a cell of the subject.

Some aspects of the present disclosure are directed to methods of increasing a binding affinity of an antigen to an HLA molecule on a cell comprising modifying an HLA-binding pocket of the HLA molecule. In some aspects, the cell is in a subject in need of a therapy.

Some aspects of the present disclosure are directed to methods of increasing immunogenicity of an antigen in a subject in need of a therapy comprising modifying an HLA-binding pocket of an HLA molecule on a cell in the subject, wherein the HLA molecule is capable of binding the antigen.

Some aspects of the present disclosure are directed to methods of increasing an immune response to a therapy in a subject in need thereof comprising modifying an HLA-binding pocket of an HLA molecule in a cell of the subject. In some aspects, the therapy comprises an immunotherapy.

Some aspects of the present disclosure are directed to methods of increasing an immune response to an antigen in a subject in need thereof, comprising modifying an HLA-binding pocket of an HLA molecule in a cell of the subject, wherein the HLA molecule is capable of binding the antigen.

In some aspects, the modifying increases a binding affinity of the HLA-binding pocket to an antigen.

In some aspects, the cell is an antigen-presenting cell. In some aspects, the cell is a dendritic cell.

In some aspects, the HLA molecule is an HLA class I allele. In some aspects, the HLA molecule is an allele selected from the group consisting of HLA-A, HLA-B, HLA-C, HLA-E, HLA-F, HLA-G, HLA-K, HLA-L, and any combination thereof.

In some aspects, the HLA molecule is an allele selected from: (a) the group consisting of HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, HLA-A*34, HLA-A*36, HLA-A*43, HLA-A*66, HLA-A*68, HLA-A*69, HLA-A*74, and HLA-A*80; (b) the group consisting of HLA-B*07, HLA-B*08, HLA-B*13, HLA-B*14, HLA-B*15, HLA-B*18, HLA-B*27, HLA-B*35, HLA-B*37, HLA-B*38, HLA-B*39, HLA-B*40, HLA-B*41, HLA-B*42, HLA-B*44, HLA-B*45, HLA-B*46, HLA-B*47, HLA-B*48, HLA-B*49, HLA-B*50, HLA-B*51, HLA-B*52, HLA-B*53, HLA-B*54, HLA-B*55, HLA-B*56, HLA-B*57, HLA-B*58, HLA-B*59, HLA-B*67, HLA-B*73, HLA-B*78, HLA-B*79, HLA-B*81, HLA-B*82, and HLA-B*83; (c) the group consisting of HLA-C*05:01, HLA-C*05:03, HLA-C*05:04, HLA-C*05:05, and HLA-C*05:06; and (d) any combination thereof.

In some aspects, the HLA molecule is an allele selected from the group consisting of HLA-A*23, HLA-A*24, and HLA-A*32. In some aspects, the HLA molecule is an allele selected from the group consisting of HLA-A*23:01:01:01, HLA-A*23:01:01:02, HLA-A*23:01:01:03, HLA-A*23:01:01:04, HLA-A*23:01:01:05, HLA-A*23:01:01:06, HLA-A*23:01:01:07, HLA-A*23:01:01:08, HLA-A*23:01:01:09, HLA-A*23:01:01:10, HLA-A*23:01:01:11, HLA-A*23:01:01:12, HLA-A*23:01:01:13, HLA-A*23:01:01:14, HLA-A*23:01:01:15, HLA-A*23:01:01:16, HLA-A*23:01:01:17, HLA-A*23:01:01:18, HLA-A*23:01:01:19, HLA-A*23:01:01:20, HLA-A*23:01:01:21, HLA-A*23:01:01:22, HLA-A*23:01:01:23, HLA-A*23:01:01:24, HLA-A*23:01:01:25, HLA-A*23:01:01:26, HLA-A*23:01:02, HLA-A*23:01:03, HLA-A*23:01:04, HLA-A*23:01:05, HLA-A*23:01:06, HLA-A*23:01:07, HLA-A*23:01:08, HLA-A*23:01:09, HLA-A*23:01:10, HLA-A*23:01:11, HLA-A*23:01:12, HLA-A*23:01:13, HLA-A*23:01:14, HLA-A*23:01:15, HLA-A*23:01:16, HLA-A*23:01:17, HLA-A*23:01:18, HLA-A*23:01:19, HLA-A*23:01:20, HLA-A*23:01:21, HLA-A*23:01:22, HLA-A*23:01:23, HLA-A*23:01:24, HLA-A*23:01:25, HLA-A*23:01:26, HLA-A*23:01:27, HLA-A*23:01:28, HLA-A*23:01:29, HLA-A*23:01:30, HLA-A*23:01:31, HLA-A*23:01:32, HLA-A*23:01:33, HLA-A*23:01:34, HLA-A*23:02, HLA-A*23:03:01, HLA-A*23:03:02:01, HLA-A*23:03:02:02, HLA-A*23:04, HLA-A*23:05, HLA-A*23:06, HLA-A*23:07, HLA-A*23:08, HLA-A*23:09, HLA-A*23:10, HLA-A*23:11, HLA-A*23:12, HLA-A*23:13, HLA-A*23:14:01, HLA-A*23:14:02, HLA-A*23:15, HLA-A*23:16, HLA-A*23:17:01:01, HLA-A*23:17:01:02, HLA-A*23:17:01:03, HLA-A*23:17:02, HLA-A*23:17:03, HLA-A*23:18, HLA-A*23:19, HLA-A*23:20, HLA-A*23:21, HLA-A*23:22, HLA-A*23:23, HLA-A*23:24, HLA-A*23:25, HLA-A*23:26, HLA-A*23:27, HLA-A*23:28, HLA-A*23:29, HLA-A*23:30, HLA-A*23:31, HLA-A*23:32, HLA-A*23:33, HLA-A*23:34, HLA-A*23:35, HLA-A*23:36, HLA-A*23:37:01, HLA-A*23:37:02, HLA-A*23:38, HLA-A*23:39, HLA-A*23:40, HLA-A*23:41, HLA-A*23:42, HLA-A*23:43, HLA-A*23:44, HLA-A*23:45, HLA-A*23:46, HLA-A*23:47, HLA-A*23:48, HLA-A*23:49, HLA-A*23:50, HLA-A*23:51, HLA-A*23:52, HLA-A*23:53, HLA-A*23:54, HLA-A*23:55, HLA-A*23:56, HLA-A*23:57, HLA-A*23:58, HLA-A*23:59, HLA-A*23:60, HLA-A*23:61, HLA-A*23:62, HLA-A*23:63, HLA-A*23:64, HLA-A*23:65, HLA-A*23:66, HLA-A*23:67, HLA-A*23:68, HLA-A*23:70, HLA-A*23:71, HLA-A*23:72, HLA-A*23:73, HLA-A*23:74, HLA-A*23:75, HLA-A*23:76, HLA-A*23:77, HLA-A*23:78, HLA-A*23:79, HLA-A*23:80, HLA-A*23:81, HLA-A*23:82, HLA-A*23:83, HLA-A*23:84, HLA-A*23:85, HLA-A*23:86, HLA-A*23:87, HLA-A*23:88, HLA-A*23:89, HLA-A*23:90, HLA-A*23:91, HLA-A*23:92, HLA-A*23:93, HLA-A*23:94, HLA-A*23:95, HLA-A*23:96, HLA-A*23:97, HLA-A*23:98, HLA-A*23:99, HLA-A*23:100, HLA-A*23:101, HLA-A*23:102, HLA-A*23:103, HLA-A*23:104, HLA-A*23:105, HLA-A*23:106, HLA-A*23:107, HLA-A*23:108, HLA-A*23:109, HLA-A*23:110, HLA-A*23:111, HLA-A*23:112, HLA-A*23:113, HLA-A*23:114, HLA-A*23:115, HLA-A*23:116, and HLA-A*23:117.

In some aspects, the HLA molecule is an allele selected from the group consisting of HLA-A*24:02:01:01, HLA-A*24:02:01:02, HLA-A*24:02:01:03, HLA-A*24:02:01:04, HLA-A*24:02:01:05, HLA-A*24:02:01:06, HLA-A*24:02:01:07, HLA-A*24:02:01:08, HLA-A*24:02:01:09, HLA-A*24:02:01:10, HLA-A*24:02:01:11, HLA-A*24:02:01:12, HLA-A*24:02:01:13, HLA-A*24:02:01:14, HLA-A*24:02:01:15, HLA-A*24:02:01:16, HLA-A*24:02:01:17, HLA-A*24:02:01:18, HLA-A*24:02:01:19, HLA-A*24:02:01:20, HLA-A*24:02:01:21, HLA-A*24:02:01:22, HLA-A*24:02:01:23, HLA-A*24:02:01:24, HLA-A*24:02:01:25, HLA-A*24:02:01:26, HLA-A*24:02:01:27, HLA-A*24:02:01:28, HLA-A*24:02:01:29, HLA-A*24:02:01:30, HLA-A*24:02:01:31, HLA-A*24:02:01:32, HLA-A*24:02:01:33, HLA-A*24:02:01:34, HLA-A*24:02:01:35, HLA-A*24:02:01:36, HLA-A*24:02:01:37, HLA-A*24:02:01:38, HLA-A*24:02:01:39, HLA-A*24:02:01:40, HLA-A*24:02:01:41, HLA-A*24:02:01:42, HLA-A*24:02:01:43, HLA-A*24:02:01:44, HLA-A*24:02:01:45, HLA-A*24:02:01:46, HLA-A*24:02:01:47, HLA-A*24:02:01:48, HLA-A*24:02:01:49, HLA-A*24:02:01:50, HLA-A*24:02:01:51, HLA-A*24:02:01:52, HLA-A*24:02:01:53, HLA-A*24:02:01:54, HLA-A*24:02:01:55, HLA-A*24:02:01:56, HLA-A*24:02:01:57, HLA-A*24:02:01:58, HLA-A*24:02:01:59, HLA-A*24:02:01:60, HLA-A*24:02:01:61, HLA-A*24:02:01:62, HLA-A*24:02:01:63, HLA-A*24:02:01:64, HLA-A*24:02:01:65, HLA-A*24:02:01:66, HLA-A*24:02:01:67, HLA-A*24:02:01:68, HLA-A*24:02:01:69, HLA-A*24:02:01:70, HLA-A*24:02:01:71, HLA-A*24:02:01:72, HLA-A*24:02:01:73, HLA-A*24:02:01:74, HLA-A*24:02:01:75, HLA-A*24:02:01:76, HLA-A*24:02:01:77, HLA-A*24:02:01:78, HLA-A*24:02:01:79, HLA-A*24:02:01:80, HLA-A*24:02:01:81, HLA-A*24:02:01:82, HLA-A*24:02:01:83, HLA-A*24:02:01:84, HLA-A*24:02:01:85, HLA-A*24:02:01:86, HLA-A*24:02:01:87, HLA-A*24:02:01:88, HLA-A*24:02:01:89, HLA-A*24:02:01:90, HLA-A*24:02:01:91, HLA-A*24:02:01:92, HLA-A*24:02:01:93, HLA-A*24:02:01:94, HLA-A*24:02:01:95, HLA-A*24:02:01:96, HLA-A*24:02:01:97, HLA-A*24:02:01:98, HLA-A*24:02:01:99, HLA-A*24:02:01:100, HLA-A*24:02:01:101, HLA-A*24:02:01:102, HLA-A*24:02:01:103, HLA-A*24:02:01:104, HLA-A*24:02:01:105, HLA-A*24:02:01:106, HLA-A*24:02:01:107, HLA-A*24:02:01:108, HLA-A*24:02:01:109, HLA-A*24:02:01:110, HLA-A*24:02:02, HLA-A*24:02:03, HLA-A*24:02:04, HLA-A*24:02:05, HLA-A*24:02:06, HLA-A*24:02:07, HLA-A*24:02:08, HLA-A*24:02:09, HLA-A*24:02:10, HLA-A*24:02:11, HLA-A*24:02:12, HLA-A*24:02:13, HLA-A*24:02:14, HLA-A*24:02:15, HLA-A*24:02:16, HLA-A*24:02:17, HLA-A*24:02:18, HLA-A*24:02:19, HLA-A*24:02:20, HLA-A*24:02:21, HLA-A*24:02:22, HLA-A*24:02:23, HLA-A*24:02:24, HLA-A*24:02:25, HLA-A*24:02:26, HLA-A*24:02:27, HLA-A*24:02:28, HLA-A*24:02:29, HLA-A*24:02:30, HLA-A*24:02:31, HLA-A*24:02:32, HLA-A*24:02:33, HLA-A*24:02:34, HLA-A*24:02:35, HLA-A*24:02:36, HLA-A*24:02:37, HLA-A*24:02:38, HLA-A*24:02:39, HLA-A*24:02:40:01, HLA-A*24:02:40:02, HLA-A*24:02:41, HLA-A*24:02:42, HLA-A*24:02:43, HLA-A*24:02:44, HLA-A*24:02:45, HLA-A*24:02:46, HLA-A*24:02:47, HLA-A*24:02:48, HLA-A*24:02:49, HLA-A*24:02:50, HLA-A*24:02:51, HLA-A*24:02:52, HLA-A*24:02:53, HLA-A*24:02:54, HLA-A*24:02:55, HLA-A*24:02:56, HLA-A*24:02:57, HLA-A*24:02:58, HLA-A*24:02:59, HLA-A*24:02:60, HLA-A*24:02:61, HLA-A*24:02:62, HLA-A*24:02:63, HLA-A*24:02:64, A*24:02:69, HLA-A*24:02:70, HLA-A*24:02:71, HLA-A*24:02:72, HLA-A*24:02:73, HLA-A*24:02:74, HLA-A*24:02:75, HLA-A*24:02:76, HLA-A*24:02:77, HLA-A*24:02:78, HLA-A*24:02:79, HLA-A*24:02:80, HLA-A*24:02:81, HLA-A*24:02:82, HLA-A*24:02:83, HLA-A*24:02:84, HLA-A*24:02:85, HLA-A*24:02:86, HLA-A*24:02:87, HLA-A*24:02:88, HLA-A*24:02:89, HLA-A*24:02:90, HLA-A*24:02:91, HLA-A*24:02:92, HLA-A*24:02:93, HLA-A*24:02:94, HLA-A*24:02:95, HLA-A*24:02:96, HLA-A*24:02:97, HLA-A*24:02:98, HLA-A*24:02:99, HLA-A*24:02:100, HLA-A*24:02:101, HLA-A*24:02:102:01, HLA-A*24:02:102:02, HLA-A*24:02:103, HLA-A*24:02:104, HLA-A*24:02:105, HLA-A*24:02:106, HLA-A*24:02:107, HLA-A*24:02:108, HLA-A*24:02:109, HLA-A*24:02:110, HLA-A*24:02:111, HLA-A*24:02:112, HLA-A*24:02:113, HLA-A*24:02:114, HLA-A*24:02:115:01, HLA-A*24:02:115:02, HLA-A*24:02:116, HLA-A*24:02:117, HLA-A*24:02:118, HLA-A*24:02:119, HLA-A*24:02:120, HLA-A*24:02:121, HLA-A*24:02:122, HLA-A*24:02:123, HLA-A*24:02:124, HLA-A*24:02:125, HLA-A*24:02:126, HLA-A*24:02:127, HLA-A*24:02:128, HLA-A*24:02:129, HLA-A*24:02:130, HLA-A*24:02:131, HLA-A*24:02:132, HLA-A*24:02:133, HLA-A*24:02:134, HLA-A*24:02:135, HLA-A*24:02:136, HLA-A*24:02:137, HLA-A*24:02:138, HLA-A*24:02:139, HLA-A*24:02:140, HLA-A*24:02:141, HLA-A*24:02:142, HLA-A*24:02:143, HLA-A*24:02:144, HLA-A*24:02:145, HLA-A*24:02:146, HLA-A*24:02:147, HLA-A*24:02:148, HLA-A*24:02:149, HLA-A*24:03:01:01, HLA-A*24:03:01:02, HLA-A*24:03:01:03, HLA-A*24:03:02, HLA-A*24:03:03, HLA-A*24:03:04, HLA-A*24:04, HLA-A*24:05:01, HLA-A*24:05:02, HLA-A*24:06, HLA-A*24:07:01:01, HLA-A*24:07:01:02, HLA-A*24:07:01:03, HLA-A*24:07:02, HLA-A*24:07:03, HLA-A*24:07:04, HLA-A*24:08, HLA-A*24:09, HLA-A*24:10:01:01, HLA-A*24:10:01:02, HLA-A*24:10:02, HLA-A*24:11, HLA-A*24:13:01, HLA-A*24:13:02, HLA-A*24:14:01:01, HLA-A*24:14:01:02, HLA-A*24:14:01:03, HLA-A*24:14:01:04, HLA-A*24:15, HLA-A*24:17:01:01, HLA-A*24:17:01:02, HLA-A*24:18, HLA-A*24:19, HLA-A*24:20:01:01, HLA-A*24:20:01:02, HLA-A*24:20:02, HLA-A*24:21:01, HLA-A*24:21:02, HLA-A*24:21:03, HLA-A*24:22, HLA-A*24:23, HLA-A*24:24, HLA-A*24:25, HLA-A*24:26, HLA-A*24:27, HLA-A*24:28, HLA-A*24:29, HLA-A*24:30, HLA-A*24:31, HLA-A*24:32, HLA-A*24:33, HLA-A*24:34, HLA-A*24:35, HLA-A*24:36, HLA-A*24:37, HLA-A*24:38, HLA-A*24:39, HLA-HLA-A*24:46, HLA-A*24:47, HLA-A*24:48, HLA-A*24:49, HLA-A*24:50, HLA-A*24:51, HLA-A*24:52, HLA-A*24:53, HLA-A*24:54, HLA-A*24:55, HLA-A*24:56:01, HLA-A*24:56:02, HLA-A*24:57, HLA-A*24:58, HLA-A*24:59, HLA-A*24:60, HLA-A*24:61, HLA-A*24:62, HLA-A*24:63, HLA-A*24:64, HLA-A*24:66, HLA-A*24:67, HLA-A*24:68, HLA-A*24:69, HLA-A*24:70, HLA-A*24:71, HLA-A*24:72, HLA-A*24:73, HLA-A*24:74:01, HLA-A*24:74:02, HLA-A*24:75, HLA-A*24:76, HLA-A*24:77, HLA-A*24:78, HLA-A*24:79, HLA-A*24:80, HLA-A*24:81, HLA-A*24:82, HLA-A*24:83, HLA-A*24:84, HLA-A*24:85, HLA-A*24:86, HLA-A*24:87, HLA-A*24:88, HLA-A*24:89, HLA-A*24:90:01, HLA-A*24:90:02, HLA-A*24:91, HLA-A*24:92, HLA-A*24:93, HLA-A*24:94, HLA-A*24:95, HLA-A*24:96, HLA-A*24:97, HLA-A*24:98, HLA-A*24:99, HLA-A*24:100, HLA-A*24:101, HLA-A*24:102, HLA-A*24:103, HLA-A*24:104, HLA-A*24:105, HLA-A*24:106, HLA-A*24:107, HLA-A*24:108, HLA-A*24:109, HLA-A*24:110, HLA-A*24:111, HLA-A*24:112, HLA-A*24:113, HLA-A*24:114, HLA-A*24:115, HLA-A*24:116, HLA-A*24:117, HLA-A*24:118, HLA-A*24:119, HLA-A*24:120, HLA-A*24:121, HLA-A*24:122, HLA-A*24:123, HLA-A*24:124, HLA-A*24:125, HLA-A*24:126, HLA-A*24:127, HLA-A*24:128, HLA-A*24:129, HLA-A*24:130, HLA-A*24:131, HLA-A*24:132, HLA-A*24:133, HLA-A*24:134, HLA-A*24:135:01, HLA-A*24:135:02, HLA-A*24:136, HLA-A*24:137, HLA-A*24:138, HLA-A*24:139, HLA-A*24:140, HLA-A*24:141, HLA-A*24:142:01, HLA-A*24:142:02, HLA-A*24:143, HLA-A*24:144, HLA-A*24:145, HLA-A*24:146, HLA-A*24:147, HLA-A*24:148, HLA-A*24:149, HLA-A*24:150, HLA-A*24:151, HLA-A*24:152, HLA-A*24:153, HLA-A*24:154, HLA-A*24:155, HLA-A*24:156, HLA-A*24:157, HLA-A*24:158, HLA-A*24:159, HLA-A*24:160, HLA-A*24:161, HLA-A*24:162, HLA-A*24:163, HLA-A*24:164, HLA-A*24:165, HLA-A*24:166, HLA-A*24:167, HLA-A*24:168, HLA-A*24:169, HLA-A*24:170, HLA-A*24:171, HLA-A*24:172:01, HLA-A*24:172:02, HLA-A*24:173, HLA-A*24:174, HLA-A*24:175, HLA-A*24:176, HLA-A*24:177, HLA-A*24:178, HLA-A*24:179, HLA-A*24:180, HLA-A*24:181, HLA-A*24:182, HLA-A*24:183, HLA-A*24:184, HLA-A*24:185, HLA-A*24:186, HLA-A*24:187, HLA-A*24:188, HLA-A*24:189, HLA-A*24:190, HLA-A*24:191, HLA-A*24:192, HLA-A*24:193, HLA-A*24:194, HLA-A*24:195, HLA-A*24:196, HLA-A*24:197, HLA-A*24:198, HLA-A*24:199, HLA-A*24:200, HLA-A*24:201, HLA-A*24:202, HLA-A*24:203, HLA-HLA-A*24:208:01, HLA-A*24:208:02:01, HLA-A*24:208:02:02, HLA-A*24:209, HLA-A*24:210, HLA-A*24:212, HLA-A*24:213, HLA-A*24:214, HLA-A*24:215, HLA-A*24:216, HLA-A*24:217, HLA-A*24:218, HLA-A*24:219, HLA-A*24:220, HLA-A*24:221, HLA-A*24:222, HLA-A*24:223, HLA-A*24:224, HLA-A*24:225:01, HLA-A*24:225:02, HLA-A*24:226:01, HLA-A*24:226:02, HLA-A*24:227, HLA-A*24:228, HLA-A*24:229, HLA-A*24:230, HLA-A*24:231, HLA-A*24:232, HLA-A*24:233, HLA-A*24:234, HLA-A*24:235, HLA-A*24:236, HLA-A*24:237, HLA-A*24:238, HLA-A*24:239, HLA-A*24:240, HLA-A*24:241, HLA-A*24:242, HLA-A*24:243, HLA-A*24:244, HLA-A*24:245, HLA-A*24:246, HLA-A*24:247, HLA-A*24:248, HLA-A*24:249, HLA-A*24:250, HLA-A*24:251, HLA-A*24:252, HLA-A*24:253, HLA-A*24:254, HLA-A*24:255, HLA-A*24:256, HLA-A*24:257, HLA-A*24:258, HLA-A*24:259, HLA-A*24:260, HLA-A*24:261, HLA-A*24:262, HLA-A*24:263, HLA-A*24:264, HLA-A*24:265, HLA-A*24:266, HLA-A*24:267, HLA-A*24:268, HLA-A*24:269, HLA-A*24:270, HLA-A*24:271, HLA-A*24:272, HLA-A*24:273, HLA-A*24:274, HLA-A*24:275, HLA-A*24:276, HLA-A*24:277, HLA-A*24:278, HLA-A*24:279, HLA-A*24:280, HLA-A*24:281, HLA-A*24:282, HLA-A*24:283, HLA-A*24:284, HLA-A*24:285, HLA-A*24:286, HLA-A*24:287, HLA-A*24:288, HLA-A*24:289, HLA-A*24:290, HLA-A*24:291, HLA-A*24:292, HLA-A*24:293, HLA-A*24:294, HLA-A*24:295, HLA-A*24:296, HLA-A*24:297, HLA-A*24:298, HLA-A*24:299, HLA-A*24:300, HLA-A*24:301, HLA-A*24:302, HLA-A*24:303, HLA-A*24:304, HLA-A*24:305, HLA-A*24:306, HLA-A*24:307, HLA-A*24:308, HLA-A*24:309, HLA-A*24:310:01, HLA-A*24:310:02, HLA-A*24:311, HLA-A*24:312, HLA-A*24:313:01, HLA-A*24:313:02, HLA-A*24:314, HLA-A*24:315, HLA-A*24:316, HLA-A*24:317, HLA-A*24:318, HLA-A*24:319, HLA-A*24:320, HLA-A*24:321, HLA-A*24:322, HLA-A*24:323, HLA-A*24:324, HLA-A*24:325, HLA-A*24:326, HLA-A*24:327, HLA-A*24:328, HLA-A*24:329, HLA-A*24:330, HLA-A*24:331, HLA-A*24:332, HLA-A*24:333, HLA-A*24:334, HLA-A*24:335, HLA-A*24:336, HLA-A*24:337, HLA-A*24:338, HLA-A*24:339, HLA-A*24:340, HLA-A*24:341, HLA-A*24:342, HLA-A*24:343, HLA-A*24:344, HLA-A*24:345, HLA-A*24:346, HLA-A*24:347:01, HLA-A*24:347:02, HLA-A*24:348, HLA-A*24:349, HLA-A*24:350, HLA-A*24:351, HLA-A*24:352, HLA-A*24:353, HLA-A*24:354, HLA-A*24:355, HLA-A*24:356, HLA-A*24:357, HLA-A*24:358, HLA-A*24:359, HLA-A*24:360, HLA-A*24:366, HLA-A*24:367, HLA-A*24:368, HLA-A*24:369, HLA-A*24:370, HLA-A*24:371, HLA-A*24:372, HLA-A*24:373, HLA-A*24:374, HLA-A*24:375, HLA-A*24:376, HLA-A*24:377, HLA-A*24:378, HLA-A*24:379, HLA-A*24:380, HLA-A*24:381, HLA-A*24:382, HLA-A*24:383, HLA-A*24:384, HLA-A*24:385, HLA-A*24:386, HLA-A*24:387, HLA-A*24:388, HLA-A*24:389, HLA-A*24:390, HLA-A*24:391, HLA-A*24:392, HLA-A*24:393, HLA-A*24:394, HLA-A*24:395, HLA-A*24:396, HLA-A*24:397, HLA-A*24:398, HLA-A*24:399, HLA-A*24:400, HLA-A*24:401, HLA-A*24:402, HLA-A*24:403, HLA-A*24:404, HLA-A*24:405, HLA-A*24:406, HLA-A*24:407, HLA-A*24:408, HLA-A*24:409, HLA-A*24:410, HLA-A*24:411, HLA-A*24:412, HLA-A*24:413, HLA-A*24:414, HLA-A*24:415, HLA-A*24:416, HLA-A*24:417, HLA-A*24:418, HLA-A*24:419, HLA-A*24:420, HLA-A*24:421, HLA-A*24:422, HLA-A*24:423, HLA-A*24:424, HLA-A*24:425, HLA-A*24:426, HLA-A*24:427, HLA-A*24:428, HLA-A*24:429, HLA-A*24:430, HLA-A*24:431:01, HLA-A*24:431:02, HLA-A*24:432, HLA-A*24:433, HLA-A*24:434, HLA-A*24:435, HLA-A*24:436, HLA-A*24:437, HLA-A*24:438, HLA-A*24:439, HLA-A*24:440, HLA-A*24:441, HLA-A*24:442, HLA-A*24:443, HLA-A*24:444, HLA-A*24:445, HLA-A*24:446, HLA-A*24:447, HLA-A*24:448, HLA-A*24:449, HLA-A*24:450, HLA-A*24:451, HLA-A*24:452, HLA-A*24:453, HLA-A*24:454, HLA-A*24:455, HLA-A*24:456, HLA-A*24:457, HLA-A*24:458, HLA-A*24:459, HLA-A*24:460:01:01, HLA-A*24:460:01:02, HLA-A*24:461, HLA-A*24:462, HLA-A*24:463, HLA-A*24:464, HLA-A*24:465, HLA-A*24:466, HLA-A*24:467, HLA-A*24:468, HLA-A*24:469, HLA-A*24:470, HLA-A*24:472, HLA-A*24:473, HLA-A*24:474, HLA-A*24:475, HLA-A*24:476, HLA-A*24:477, HLA-A*24:478, HLA-A*24:479, HLA-A*24:480, HLA-A*24:481, HLA-A*24:482, HLA-A*24:483, HLA-A*24:484, HLA-A*24:485, HLA-A*24:486, HLA-A*24:487, HLA-A*24:488, HLA-A*24:489, HLA-A*24:490, HLA-A*24:491, HLA-A*24:492, HLA-A*24:493, HLA-A*24:494, HLA-A*24:495, HLA-A*24:496, HLA-A*24:497, HLA-A*24:498, HLA-A*24:499, HLA-A*24:500, HLA-A*24:501, HLA-A*24:502, HLA-A*24:503, HLA-A*24:504, HLA-A*24:505, HLA-A*24:506, HLA-A*24:507, HLA-A*24:508, HLA-A*24:509, HLA-A*24:510, HLA-A*24:511, HLA-A*24:512, HLA-A*24:513, HLA-A*24:514, HLA-A*24:515, HLA-A*24:516, HLA-A*24:517, HLA-A*24:518, HLA-A*24:519, HLA-A*24:520, HLA-A*24:521, HLA-A*24:522, HLA-A*24:523, HLA-A*24:529, HLA-A*24:530, HLA-A*24:531, HLA-A*24:532, HLA-A*24:533, HLA-A*24:534, HLA-A*24:535, HLA-A*24:536, HLA-A*24:537, HLA-A*24:538, HLA-A*24:539, HLA-A*24:540, HLA-A*24:541, HLA-A*24:542, HLA-A*24:543, HLA-A*24:544, HLA-A*24:545, HLA-A*24:546, HLA-A*24:547, HLA-A*24:548, HLA-A*24:549, HLA-A*24:550, and HLA-A*24:551.

In some aspects, the HLA molecule is an allele selected from the group consisting of HLA-A*32:01:01:01, HLA-A*32:01:01:02, HLA-A*32:01:01:03, HLA-A*32:01:01:04, HLA-A*32:01:01:05, HLA-A*32:01:01:06, HLA-A*32:01:01:07, HLA-A*32:01:01:08, HLA-A*32:01:01:09, HLA-A*32:01:01:10, HLA-A*32:01:01:11, HLA-A*32:01:01:12, HLA-A*32:01:01:13, HLA-A*32:01:01:14, HLA-A*32:01:01:15, HLA-A*32:01:01:16, HLA-A*32:01:01:17, HLA-A*32:01:01:18, HLA-A*32:01:01:19, HLA-A*32:01:01:20, HLA-A*32:01:01:21, HLA-A*32:01:01:22, HLA-A*32:01:01:23, HLA-A*32:01:01:24, HLA-A*32:01:01:25, HLA-A*32:01:01:26, HLA-A*32:01:01:27, HLA-A*32:01:01:28, HLA-A*32:01:01:29, HLA-A*32:01:01:30, HLA-A*32:01:02, HLA-A*32:01:03, HLA-A*32:01:04, HLA-A*32:01:05, HLA-A*32:01:06, HLA-A*32:01:07, HLA-A*32:01:08, HLA-A*32:01:09, HLA-A*32:01:10, HLA-A*32:01:11, HLA-A*32:01:12, HLA-A*32:01:13, HLA-A*32:01:14, HLA-A*32:01:15, HLA-A*32:01:16, HLA-A*32:01:17, HLA-A*32:01:18, HLA-A*32:01:19, HLA-A*32:01:20, HLA-A*32:01:21, HLA-A*32:01:22, HLA-A*32:01:23, HLA-A*32:01:24, HLA-A*32:01:25, HLA-A*32:01:26, HLA-A*32:01:27, HLA-A*32:01:28, HLA-A*32:01:29, HLA-A*32:01:30, HLA-A*32:01:31, HLA-A*32:01:32, HLA-A*32:01:33, HLA-A*32:01:34, HLA-A*32:01:35, HLA-A*32:01:36, HLA-A*32:01:37, HLA-A*32:01:38, HLA-A*32:01:39, HLA-A*32:01:40, HLA-A*32:01:41, HLA-A*32:01:42, HLA-A*32:01:43, HLA-A*32:01:44, HLA-A*32:01:45, HLA-A*32:01:46, HLA-A*32:01:47, HLA-A*32:02, HLA-A*32:03:01:01, HLA-A*32:03:01:02, HLA-A*32:04, HLA-A*32:05, HLA-A*32:06, HLA-A*32:07, HLA-A*32:08, HLA-A*32:09, HLA-A*32:10, HLA-A*32:11, HLA-A*32:12, HLA-A*32:13, HLA-A*32:14, HLA-A*32:15, HLA-A*32:16, HLA-A*32:17, HLA-A*32:18, HLA-A*32:19, HLA-A*32:20, HLA-A*32:21, HLA-A*32:22, HLA-A*32:23, HLA-A*32:24, HLA-A*32:25, HLA-A*32:26:01, HLA-A*32:26:02, HLA-A*32:27, HLA-A*32:28, HLA-A*32:29, HLA-A*32:30:01, HLA-A*32:30:02, HLA-A*32:31, HLA-A*32:32, HLA-A*32:33:01, HLA-A*32:33:02, HLA-A*32:33:03, HLA-A*32:34, HLA-A*32:35, HLA-A*32:36, HLA-A*32:37, HLA-A*32:38, HLA-A*32:39, HLA-A*32:40, HLA-A*32:41, HLA-A*32:42, HLA-A*32:43:01, HLA-A*32:43:02, HLA-A*32:44, HLA-A*32:45, HLA-A*32:46:01, HLA-A*32:46:02, HLA-A*32:47, HLA-A*32:48, HLA-A*32:49, HLA-A*32:50, HLA-A*32:51, HLA-A*32:52, HLA-A*32:53, HLA-A*32:54, HLA-A*32:55:01, HLA-A*32:55:02, HLA-A*32:55:03, HLA-A*32:56, HLA-A*32:57, HLA-A*32:58, HLA-A*32:59, HLA-A*32:60, HLA-A*32:61, HLA-A*32:62, HLA-A*32:63, HLA-A*32:64, HLA-A*32:65, HLA-A*32:66, HLA-A*32:67, HLA-A*32:68, HLA-A*32:69, HLA-A*32:70, HLA-A*32:71, HLA-A*32:72, HLA-A*32:73, HLA-A*32:74, HLA-A*32:75, HLA-A*32:76, HLA-A*32:77, HLA-A*32:78, HLA-A*32:79, HLA-A*32:80, HLA-A*32:81, HLA-A*32:82, HLA-A*32:83, HLA-A*32:84, HLA-A*32:85, HLA-A*32:86, HLA-A*32:87, HLA-A*32:88, HLA-A*32:89, HLA-A*32:90, HLA-A*32:91, HLA-A*32:92, HLA-A*32:93, HLA-A*32:94, HLA-A*32:95, HLA-A*32:96, HLA-A*32:97, HLA-A*32:98, HLA-A*32:99, HLA-A*32:100, HLA-A*32:101, HLA-A*32:102, HLA-A*32:103, HLA-A*32:104, HLA-A*32:105, HLA-A*32:106:01:01, HLA-A*32:106:01:02, HLA-A*32:107, HLA-A*32:108, HLA-A*32:109, HLA-A*32:110, HLA-A*32:111, HLA-A*32:112, HLA-A*32:113, HLA-A*32:114, HLA-A*32:115, HLA-A*32:116, HLA-A*32:117, HLA-A*32:118, HLA-A*32:119, HLA-A*32:120, HLA-A*32:121, HLA-A*32:122, HLA-A*32:123, HLA-A*32:124, HLA-A*32:125, HLA-A*32:126, HLA-A*32:127, HLA-A*32:128, HLA-A*32:129, HLA-A*32:130, HLA-A*32:131, HLA-A*32:132, HLA-A*32:133, HLA-A*32:134, HLA-A*32:135, HLA-A*32:136, HLA-A*32:137, HLA-A*32:138, HLA-A*32:139, HLA-A*32:140, HLA-A*32:141, HLA-A*32:142, HLA-A*32:143, HLA-A*32:144, HLA-A*32:145, HLA-A*32:146, HLA-A*32:147, HLA-A*32:148, HLA-A*32:149, HLA-A*32:150, HLA-A*32:151, HLA-A*32:152, HLA-A*32:153, HLA-A*32:154, HLA-A*32:155, and HLA-A*32:156. In some aspects, the HLA-binding pocket is A pocket, B pocket, C pocket, D pocket, E pocket, F pocket, or any combination thereof. In some aspects, the binding pocket is F pocket. In some aspects, more than one binding pocket is modified.

In some aspects, the modifying increases the display of the antigen on the surface of the cell. In some aspects, the modifying increases an antigen-specific T cell response. In some aspects, the antigen is a tumor antigen. In some aspects, the modifying increases expansion of tumor-antigen specific T cells.

The method of any one of claims 1 to 25, wherein the modifying comprises mutating an amino acid in the HLA-binding pocket of the HLA molecule. In some aspects, the mutating comprises an amino acid substitution. In some aspects, the modifying comprises an amino acid substitution, wherein the amino acid to be replaced is an alanine. In some aspects, the new amino acid that replaces the original amino acid is selected from the group consisting of leucine, valine, isoleucine, methionine, phenylalanine, tyrosine, and tryptophan. In some aspects, the new amino acid that replaces the original amino acid is a leucine. In some aspects, the modification comprises mutating an amino acid residue selected from amino acid residue 79, amino acid residue 80, amino acid residue 81, amino acid residue 82, or amino acid residue 83, wherein the positions correspond to the amino acid sequence set forth in SEQ ID NO: 1. In some aspects, the modification comprises mutating amino acid residue 81, corresponding to the amino acid sequence set forth in SEQ ID NO: 1. In some aspects, the modification comprises a substitution of the amino acid residue at position 81, corresponding to the amino acid sequence set forth in SEQ ID NO: 1. In some aspects, the modification comprises an A81L substitution, corresponding to the amino acid sequence set forth in SEQ ID NO: 1.

In some aspects, the modifying is performed by a gene editing tool. In some aspects, the gene editing tool comprises CRISPR technology, a zinc finger nuclease, TALEN, a shRNA, siRNA, miRNA, antisense oligonucleotides, meganuclease, restriction endonuclease, or any combination thereof.

In some aspects, the method further comprises administering an immunotherapy to the subject.

In some aspects, the immunotherapy comprises an immune cell therapy. In some aspects, the immune cell therapy comprises administering a T cell, an NK cell, a tumor-infiltrating lymphocyte, or any combination thereof. In some aspects, the immune cell therapy comprises administering an engineered T cell, wherein the engineered T cell comprises a nucleic acid molecule encoding a chimeric antigen receptor (CAR), a heterologous T cell receptor (TCR), an engineered TCR, or any combination thereof.

In some aspects, the immunotherapy comprises administering a checkpoint inhibitor. In some aspects, the immunotherapy comprises administering a PD-1 inhibitor, a PD-L1 inhibitor, a CTLA-4 inhibitor, a LAG3 inhibitor, a TIM3 inhibitor, or any combination thereof. In some aspects, the immunotherapy comprises administering an antibody or an antigen-binding portion thereof that specifically binds PD-1 and inhibits the interaction of PD-1 to PD-L1. In some aspects, the antibody or an antigen-binding portion thereof that specifically binds PD-1 and inhibits the interaction of PD-1 to PD-L1 is selected from nivolumab and pembrolizumab. In some aspects, the immunotherapy comprises administering an antibody or an antigen-binding portion thereof that specifically binds and inhibits CTLA-4. In some aspects, the antibody or an antigen-binding portion thereof that specifically binds and inhibits CTLA-4 is selected from ipilimumab and tremelimumab.

In some aspects, the immunotherapy comprises administering an antibody or an antigen-binding portion thereof that specifically binds and inhibits LAG3. In some aspects, the immunotherapy comprises a cancer vaccine.

In some aspects, the subject is afflicted with a cancer. In some aspects, the cancer is selected from the group consisting of melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers.

Some aspects of the present disclosure are directed to an HLA molecule comprising a modified HLA-binding pocket.

In some aspects, the HLA molecule is an HLA class I allele. In some aspects, the HLA molecule is an allele selected from the group consisting of HLA-A, HLA-B, HLA-C, HLA-E, HLA-F, HLA-G, HLA-K, HLA-L, and any combination thereof. In some aspects, the HLA molecule is an allele selected from: (a) the group consisting of HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, HLA-A*34, HLA-A*36, HLA-A*43, HLA-A*66, HLA-A*68, HLA-A*69, HLA-A*74, and HLA-A*80; (b) the group consisting of HLA-B*07, HLA-B*08, HLA-B*13, HLA-B*14, HLA-B*15, HLA-B*18, HLA-B*27, HLA-B*35, HLA-B*37, HLA-B*38, HLA-B*39, HLA-B*40, HLA-B*41, HLA-B*42, HLA-B*44, HLA-B*45, HLA-B*46, HLA-B*47, HLA-B*48, HLA-B*49, HLA-B*50, HLA-B*51, HLA-B*52, HLA-B*53, HLA-B*54, HLA-B*55, HLA-B*56, HLA-B*57, HLA-B*58, HLA-B*59, HLA-B*67, HLA-B*73, HLA-B*78, HLA-B*79, HLA-B*81, HLA-B*82, and HLA-B*83; (c) the group consisting of HLA-C*05:01, HLA-C*05:03, HLA-C*05:04, HLA-C*05:05, and HLA-C*05:06; and (d) any combination thereof. In some aspects, the HLA molecule is an allele selected from the group consisting of HLA-A*23, HLA-A*24, HLA-A*25, HLA-A*31, and HLA-A*32. In some aspects, the HLA molecule is an HLA-A*24 allele.

In some aspects, the HLA-binding pocket is A pocket, B pocket, C pocket, D pocket, E pocket, F pocket, or any combination thereof. In some aspects, the HLA-binding pocket is F pocket.

In some aspects, the HLA molecule comprises more than one modified HLA-binding pocket.

In some aspects, the HLA molecule has a higher affinity for an antigen than an HLA molecule comprising an unmodified HLA-binding pocket. In some aspects, the antigen is a tumor antigen.

In some aspects, the modified HLA-binding pocket comprises an amino acid substitution relative to an unmodified HLA-binding pocket. In some aspects, the original amino acid to be replaced by the substitution is an alanine. In some aspects, the new amino acid that replaces the original amino acid is selected from the group consisting of leucine, valine, isoleucine, methionine, phenylalanine, tyrosine, and tryptophan. In some aspects, the new amino acid that replaces the original amino acid is a leucine.

In some aspects, the HLA-binding pocket comprises an amino acid substitution at a residue selected from amino acid residue 79, amino acid residue 80, amino acid residue 81, amino acid residue 82, or amino acid residue 83, wherein the positions correspond to the amino acid sequence set forth in SEQ ID NO: 1. In some aspects, the HLA-binding pocket comprises an amino acid substitution at amino acid residue 81, corresponding to the amino acid sequence set forth in SEQ ID NO: 1. In some aspects, the HLA-binding pocket comprises an A81L substitution, corresponding to the amino acid sequence set forth in SEQ ID NO: 1.

Some aspects of the present disclosure are directed to a nucleic acid or a set of nucleic acids encoding an HLA molecule disclosed herein. Some aspects of the present disclosure are directed to a vector or a set of vectors comprising a nucleic acid disclosed herein. In some aspects, the vector is a viral vector.

Some aspects of the present disclosure are directed to an HLA-antigen complex comprising an HLA disclosed herein and an antigen. In some aspects, the antigen is a tumor antigen.

Some aspects of the present disclosure are directed to an antigen-presenting cell comprising an HLA molecule disclosed herein, a nucleic acid or a set of nucleic acids disclosed herein, a vector or a set of vectors disclosed herein, or an HLA-antigen complex disclosed herein. In some aspects, the antigen-presenting cell is a dendritic cell. In some aspects, the antigen-presenting cell is an artificial antigen-presenting cell.

Some aspects of the present disclosure are directed to a pharmaceutical composition comprising an HLA molecule disclosed herein, a nucleic acid or a set of nucleic acids disclosed herein, a vector or a set of vectors disclosed herein, an HLA-antigen complex disclosed herein, or an antigen-presenting cell disclosed herein and a pharmaceutically acceptable excipient.

Some aspects of the present disclosure are directed to a vaccine comprising an HLA molecule disclosed herein, a nucleic acid or a set of nucleic acids disclosed herein, a vector or a set of vectors disclosed herein, an HLA-antigen complex disclosed herein, or an antigen-presenting cell disclosed herein.

Some aspects of the present disclosure are directed to a method of treating a subject in need thereof comprising administering to the subject an HLA molecule disclosed herein, a nucleic acid or a set of nucleic acids disclosed herein, a vector or a set of vectors disclosed herein, an HLA-antigen complex disclosed herein, an antigen-presenting cell disclosed herein, a pharmaceutical composition disclosed herein, or a vaccine disclosed herein.

In some aspects, the method further comprises administering to the subject an immunotherapy.

In some aspects, the immunotherapy comprises an immune cell therapy. In some aspects, the immune cell therapy comprises administering a T cell, an NK cell, a tumor-infiltrating lymphocyte, or any combination thereof. In some aspects, the immune cell therapy comprises administering an engineered T cell, wherein the engineered to T cell comprises a nucleic acid molecule encoding a chimeric antigen receptor CAR, a heterologous T cell receptor (TCR), an engineered TCR, or any combination thereof.

In some aspects, the immunotherapy comprises administering a checkpoint inhibitor. The method of any one of claims 80 to 84, wherein the immunotherapy comprises administering a PD-1 inhibitor, a PD-L1 inhibitor, a CTLA-4 inhibitor, a LAG3 inhibitor, a TIM3 inhibitor, or any combination thereof. In some aspects, the immunotherapy comprises administering an antibody or an antigen-binding portion thereof that specifically binds PD-1 and inhibits the interaction of PD-1 to PD-L1, an antibody or an antigen-binding portion thereof that specifically binds and inhibits CTLA-4, an antibody or an antigen-binding portion thereof that specifically binds and inhibits LAG3, or any combination thereof. In some aspects, (i) the antibody or an antigen-binding portion thereof that specifically binds PD-1 and inhibits the interaction of PD-1 to PD-L1 is selected from nivolumab and pembrolizumab, (ii) the antibody or an antigen-binding portion thereof that specifically binds and inhibits CTLA-4 is selected from ipilimumab and tremelimumab. (iii) any combination of (i) and (ii).

In some aspects, the subject is afflicted with a cancer. In some aspects, the cancer is selected from the group consisting of melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers.

Some aspects of the present disclosure are directed to methods of enriching a population of T cells obtained from a human subject comprising contacting the T cells with an HLA molecule disclosed herein, an HLA-antigen complex disclosed herein, or an antigen-presenting cell disclosed herein.

### BRIEF DESCRIPTION OF THE DRAWINGS

FIG. 1 provides an amino acid sequence alignment of the α1 domain of the most prevalent HLA class I alleles expressed within the population. FIG. 1 demonstrates that the amino acid sequence of HLA-A*24:02 differs from other HLA class I alleles at position 81-83. The square represents amino acid residues at position 81 to 83.

FIGS. 2A and 2B graphical representations of peptide-HLA complex surface formation. FIG. 2A provides surface expression of ANGER, HLA class I, and HLA-A*24:02 in T2 cells transduced with the full-length HLA-A*24:02 genes tagged with ΔNGFR and analyzed by flow cytometry followed by staining with indicated antibodies (open curve) or isotype control (filled curve). FIG. 2B provides mean fluorescence intensity (MFI) of biotinylated peptides with streptavidin-PE on the T2-HLA-A*24:02 (wild-type or A81L) cells. Peptides used are shown in Table 1. The B7-restricted HPV E7₅₋₁₃ and C7-restricted MART1₅₁₋₆₁ peptides were used as irrelevant controls. For data analysis, the MFI of the samples to the nonbiotinylated peptide was subtracted from the MFI of the samples to the biotinylated peptide. P values were determined by repeated measures one-way analysis of variance (ANOVA) with Tukey's multiple comparisons test for each peptide (F=1128 (Adenovirus 5 Hexon), 45.78 (CMV-IE1), 453.3 (CMV pp65), 484.2 (HIV env), 20.08 (EpCAM), and 430.2 (KM-HN-1); degrees of freedom=11).

TABLE 1
Peptides used for peptide-HLA binding assay.

| | | | Peptide sequence | Peptide sequence |
|---|---|---|---|---|
| No. | Protein | Position | (biotinylated) | (nonbiotinylated) |
| 1 | CMV IE1 | 248 | AYAQKIFK(-biotin) | AYAQKIFKI |
| 2 | Adenovirus 5 | 37 | TYFSLNNK(-biotin)F | TYFSLNNKF |
| | Hexon | | | |
| 3 | CMV pp65 | 113 | VYALPLK(-biotin)ML | VYALPLKML |
| 4 | HIV env | 67 | RYLK(-biotin)DQQLL | RYLKDQQLL |
| 5 | EpCAM | 173 | RYQLDPK(-biotin)FI | RYQLDPKFI |
| 6 | KM-HN-1 | 770 | EYLSLSDK(-biotin)I | EYLSLSDKI |
| 7 | HPV E7 | 5 | KPTLK(-biotin)EYVL | KPTLKEYVL |
| 8 | MART1 | 51 | RNGYRALMDK(-biotin)S | RNGYRALMDKS |

FIGS. 3A-3D illustrate that position 81 is critical for the surface expression of HLA-A*02:01. FIGS. 3A and 3B are graphical representations of surface (FIG. 3A) and intracellular (FIG. 3B) expression of endogenous β2m and HLA class I molecules in T2 cells, and β2m, and HLA class I derived from transduced β2m-HLA-A*02:01 (wild-type or L81A) in T2/β2mKO cells, analyzed via flow cytometry following staining with an anti-β2m and HLA-class I mAbs (open curve) and an isotype control (filled curve). FIG. 3A provides surface expression, and FIG. 3B provides intracellular expression after fixation and permeabilization. FIGS. 3C and 3D show Jurkat 76/CD8 cells transduced with A2/NY-ESO-1₁₅₇₋₁₆₅ TCR (clone 1G4LY; FIG. 3C) or A2/gp100₁₅₄₋₁₆₂ TCR (FIG. 3D) were used as responder cells in IFN-γ ELISPOT analysis. The indicated T2 cells pulsed with 10 μg/ml heteroclitic NY-ESO-1₁₅₇₋₁₆₅, gp100₁₅₄₋₁₆₂, or HIV pol₄₇₆₋₄₈₄ control peptide were utilized as stimulator cells. The data shown represent the mean±SD of experiments performed in triplicate. **P<0.01, ***P<0.001 (two-tailed Welch's t tests).

FIGS. 4A and 4B are graphical representations illustrating that HLA-A*24:02 (A81L) enhances the presentation of antigens to T cells. FIG. 4A provides Jurkat 76/CD8 cells individually transduced with the indicated HLA-A*24:02-restricted WT1₂₃₅₋₂₄₃ TCR used as responder cells in IL-2 ELISPOT analysis. T2 cells transduced with HLA-A*24:02 (wild-type or A81L) pulsed with graded concentrations of A24/heteroclitic WT1₂₃₅₋₂₄₃ peptide were cocultured with T cells in the presence of peptide (FIG. 4A, top row) or T2 cells pulsed with peptide (FIG. 4A, bottom row). Secretion of IL-2 relative to the maximal response was analyzed. FIG. 4B provides primary T cells transduced with HLA-A*24/gp100-intron4₁₇₀₋₁₇₈ TCR used as responder cells in IFN-γ ELISPOT analysis. T2 or T2 transduced with HLA-A*24:02 (wild-type or A81L) cells were pulsed with the indicated gp100-intron4 peptide in serum-free media were utilized as stimulator cells. The HIV env₅₈₄₋₅₉₂ peptide and untransduced primary T cells were employed as negative controls. *P<0.05, **P<0.01 (two-tailed Welch's t tests).

FIGS. 5A-5C are graphical representations demonstrating peptide-loaded HLA-A*24:02 (A81L) multimers can be used to stain low affinity TCRs. FIGS. 5A and 5B provide peptide-exchange efficiency in soluble monomeric HLA-A*24:02 wild-type (FIG. 5A) or with A81L substitution (FIG. 5B) measured by peptide competition binding assay and ELISA. The HLA-A*24:02-restricted peptides employed are shown in Table 1. HLA-B*18-restricted MAGE-A3₁₆₇₋₁₇₆ (No. 65), HLA-B*27-restricted VEGF (No. 66), and HLA-C*16-restricted MAGE-A4₂₉₃₋₃₀₁ (No. 67) peptides were used as irrelevant controls. The data shown represent the mean±SD of experiments performed in triplicate. FIG. 5C provides HLA-A*24:02 multimers with A81L substitution efficiently stain low affinity the cognate TCRs. Jurkat 76/CD8 cells transduced with three different A24/WT1 TCRs or A24/gp100-intron4₁₇₀₋₁₇₈ TCR as control, were stained with the HLA-A*24:02^{Q115E}-K^{b}/wild-type WT1 (FIG. 5C, top row) or HLA-A*24:02^{Q115E/A81L}-K^{b}/wild-type WT1 (FIG. 5C, bottom row) multimers. The percentage of multimer⁺ cells in CD8⁺ T cells is shown.

FIGS. 6A-6C are graphical representations demonstrating aAPC/HLA-A*24:02 (A81L) enhances expansion of tumor-antigen specific T cells. CD8⁺ T cells isolated from melanoma TILs were stimulated with the aAPC/HLA-A*24:02 (wild-type or A81L) pulsed with 10 μg/ml gp100-intron4₁₇₀₋₁₇₈ or HTLV-1 tax₃₀₁₋₃₀₉ peptide. FIG. 6A provides staining with the indicated multimers before stimulation (day 0) and after 14 days stimulation. Gates indicate percentage multimer⁺ CD8⁺ T cells. FIG. 6B provides cumulative frequency of gp100-intron4₁₇₀₋₁₇₈-multimer⁺ CD8⁺ T cells after 14 days of expansion cultures. FIG. 6C provides cumulative fold-change of gp100-intron4₁₇₀₋₁₇₈-multimer⁺ CD8⁺ T cells after 14 days of expansion cultures. The data shown represent the mean±SD. *P<0.05 (paired test).

FIGS. 7A and 7B are protein models showing A81L substitution in HLA-A*24:02 peptide complex. FIGS. 7A and 7B show a top view of the peptide-binding grove of HLA-A*24:02 complexed with Flu PB1₄₉₈₋₅₀₅ peptide including a position 81 side chain (RCSB PDB: 4F7T). The models with alanine at position 81 (A81) (FIG. 7A) and leucine at position 81 (81L) (FIG. 7B) are shown.

### DETAILED DESCRIPTION OF THE DISCLOSURE

Some aspects of the present disclosure are directed to methods of conditioning a subject in need of a therapy comprising modifying a human leukocyte antigen (HLA)-binding pocket expressed on a cell of the subject. Some aspects of the present disclosure are directed to methods of enhancing an immune response in a subject in need thereof, comprising modifying an HLA-binding binding pocket of an HLA molecule expressed on a cell of the subject. Other aspects of the present disclosure are directed to methods of increasing a binding affinity of an antigen to an HLA molecule on a cell comprising modifying an HLA-binding pocket of the HLA molecule. In some aspects, the binding pocket of the HLA is modified to increase binding affinity of the HLA to an antigen. Further aspects of the present disclosure are directed to engineered antigen-presenting cells, which comprise a binding pocket that is modified to increase the affinity of the HLA to an antigen.

Some aspects of the present disclosure are further directed to methods of identifying novel T cell receptors (TCRs) that are capable of binding a target antigen-HLA complex, comprising (i) contacting a target antigen with an engineered antigen-presenting cell, wherein the engineered antigen-presenting cell comprises a binding pocket that is modified to increase the affinity of the HLA to an antigen, and (ii) contacting a plurality of TCRs with the target antigen-HLA complex.

Other aspects of the present disclosure are directed to a vaccine comprising an engineered antigen-presenting cell complexed with a target antigen, wherein the engineered antigen-presenting cell comprises a binding pocket that is modified to increase the affinity of the HLA to an antigen.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “T cell receptor” (TCR), as used herein, refers to a heteromeric cell-surface receptor capable of specifically interacting with a target antigen. As used herein, “TCR” includes but is not limited to naturally occurring and non-naturally occurring TCRs; full-length TCRs and antigen binding portions thereof; chimeric TCRs; TCR fusion constructs; and synthetic TCRs. In human, TCRs are expressed on the surface of T cells, and they are responsible for T cell recognition and targeting of antigen presenting cells. Antigen presenting cells (APCs) display fragments of foreign proteins (antigens) complexed with the major histocompatibility complex (MHC; also referred to herein as complexed with an HLA molecule, e.g., an HLA class 1 molecule). A TCR recognizes and binds to the antigen:HLA complex and recruits CD3 (expressed by T cells), activating the TCR. The activated TCR initiates downstream signaling and an immune response, including the destruction of the EPC.

In general, a TCR can comprise two chains, an alpha chain and a beta chain (or less commonly a gamma chain and a delta chain), interconnected by disulfide bonds. Each chain comprises a variable domain (alpha chain variable domain and beta chain variable domain) and a constant region (alpha chain constant region and beta chain constant region). The variable domain is located distal to the cell membrane, and the variable domain interacts with an antigen. The constant region is located proximal to the cell membrane. A TCR can further comprises a transmembrane region and a short cytoplasmic tail. As used herein, the term “constant region” encompasses the transmembrane region and the cytoplasmic tail, when present, as well as the traditional “constant region.”

The variable domains can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, FR4. Each variable domain contains a binding domain that interacts with an antigen. Though all three CDRs on each chain are involved in antigen binding, CDR3 is believed to be the primary antigen binding region. CDR1 is also interacts with the antigen, while CD2 is believed to primarily recognize the HLA complex.

Where not expressly stated, and unless the context indicates otherwise, the term “TCR” also includes an antigen-binding fragment or an antigen-binding portion of any TCR disclosed herein, and includes a monovalent and a divalent fragment or portion, and a single chain TCR. The term “TCR” is not limited to naturally occurring TCRs bound to the surface of a T cell. As used herein, the term “TCR” further refers to a TCR described herein that is expressed on the surface of a cell other than a T cell (e.g., a cell that naturally expresses or that is modified to express CD3, as described herein), or a TCR described herein that is free from a cell membrane (e.g., an isolated TCR or a soluble TCR).

An “antigen binding molecule,” “portion of a TCR,” or “TCR fragment” refers to any portion of an TCR less than the whole. An antigen binding molecule can include the antigenic complementarity determining regions (CDRs).

An “antigen” refers to any molecule, e.g., a peptide, that provokes an immune response or is capable of being bound by a TCR. An “epitope,” as used herein, refers to a portion of a polypeptide that provokes an immune response or is capable of being bound by a TCR. The immune response may involve either antibody production, or the activation of specific immunologically-competent cells, or both. A person of skill in the art would readily understand that any macromolecule, including virtually all proteins or peptides, can serve as an antigen. An antigen and/or an epitope can be endogenously expressed, i.e. expressed by genomic DNA, or can be recombinantly expressed. An antigen and/or an epitope can be specific to a certain tissue, such as a cancer cell, or it can be broadly expressed. In addition, fragments of larger molecules can act as antigens. In one aspect, antigens are tumor antigens. An epitope can be present in a longer polypeptide (e.g., in a protein), or an epitope can be present as a fragment of a longer polypeptide. In some aspects, an epitope is complexed with a major histocompatibility complex (MHC; also referred to herein as complexed with an HLA molecule, e.g., an HLA class 1 molecule).

The term “HLA,” as used herein, refers to the human leukocyte antigen. HLA genes encode the major histocompatibility complex (MHC) proteins in humans. MHC proteins are expressed on the surface of cells, and are involved in activation of the immune response. HLA class I genes encode MHC class I molecules, which are expressed on the surface of cells in complex with peptide fragments (antigens) of self or non-self proteins. T cells expressing TCR and CD3 recognize the antigen: MHC class I complex and initiate an immune response to target and destroy antigen presenting cells displaying non-self proteins.

As used herein, an “HLA class I molecule” or “HLA class I molecule” refers to a protein product of a wild-type or variant HLA class I gene encoding an MHC class I molecule. Accordingly, “HLA class I molecule” and “MHC class I molecule” are used interchangeably herein.

The MHC Class I molecule comprises two protein chains: the alpha chain and the β2-microglobulin (β2m) chain. Human β2m is encoded by the B2M gene. The alpha chain of the MHC Class I molecule is encoded by the HLA gene complex. The HLA complex is located within the 6p21.3 region on the short arm of human chromosome 6 and contains more than 220 genes of diverse function. The HLA gene are highly variant, with over 20,000 HLA alleles and related alleles, including over 15,000 HLA Class I alleles, known in the art, encoding thousands of HLA proteins, including over 10,000 HLA Class I proteins (see, e.g., hla.alleles.org, last visited Feb. 27, 2019). There are at least three genes in the HLA complex that encode an MHC Class I alpha chain protein: HLA-A, HLA-B, and HLA-C. In addition, HLA-E, HLA-F, and HLA-G encode proteins that associate with the MHC Class I molecule.

The term “autologous” refers to any material derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some aspects, the methods of the present invention can be used to reduce the tumor size of a tumor derived from, for example, bone cancer, renal cancer, prostate cancer, breast cancer, colon cancer, lung cancer, cutaneous or intraocular malignant melanoma, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. The particular cancer can be responsive to chemo- or radiation therapy or the cancer can be refractory. A refractory cancer refers to a cancer that is not amendable to surgical intervention, and the cancer is either initially unresponsive to chemo- or radiation therapy or the cancer becomes unresponsive over time.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term “progression-free survival,” which can be abbreviated as PFS, as used herein refers to the time from the treatment date to the date of disease progression per the revised IWG Response Criteria for Malignant Lymphoma or death from any cause.

“Disease progression” or “progressive disease,” which can be abbreviated as PD, as used herein, refers to a worsening of one or more symptom associated with a particular disease. For example, disease progression for a subject afflicted with a cancer can include an increase in the number or size of one or more malignant lesions, tumor metastasis, and death.

The “duration of response,” which can be abbreviated as DOR, as used herein refers to the period of time between a subject's first objective response to the date of confirmed disease progression, per the revised IWG Response Criteria for Malignant Lymphoma, or death.

The term “overall survival,” which can be abbreviated as OS, is defined as the time from the date of treatment to the date of death.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

Other examples of analytes and cytokines of the present invention include, but are not limited to chemokine (C—C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), leukemia inhibitory factor (LIF), oncostatin M (OSM), CD154, lymphotoxin (LT) beta, 4-1BB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). T-cell receptors (TCR) differentiate T cells from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory T_{EM} cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. B-cells, on the other hand, play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

The term “genetically engineered” or “engineered” refers to a method of modifying the genome of a cell, including, but not limited to, deleting a coding or non-coding region or a portion thereof or inserting a coding region or a portion thereof. In some aspects, the cell that is modified is a lymphocyte, e.g., a T cell or a modified cell that expresses CD3, which can either be obtained from a patient or a donor. The cell can be modified to express an exogenous construct, such as, e.g., a T cell receptor (TCR) disclosed herein, which is incorporated into the cell's genome. In some aspects, the cell is modified to express CD3.

The term “modified,” as used herein refers to an alteration or engineering of a target, e.g., a polypeptide, a polynucleotide, or a cell, that changes one or more aspect of the target. For example, a “modified” HLA-binding pocket refers to an HLA-binding pocket that is altered relative to a wild-type HLA-binding pocket. In some aspect, the modification is to the structure of the target, e.g., the structure of the HLA-binding pocket. In some aspects, the modification is to the amino acid sequence of the target, e.g., the modification comprises an amino acid mutation (e.g., an amino acid substitution). The term “modified” is no intended to be limited to a particular means of altering the target. In some aspects, the modification is achieved using a gene editing tool. In some aspects, the gene editing tool is the CRISPR/Cas system, Transcription Activator-Like Effector Nucleases (TALENs), meganucleases, or a zinc-finger nuclease (ZFN) system.

The term “engineered,” when used herein in the context of a cell, refers to a cell that is modified to comprise a heterologous polynucleotide or that is genetically engineered in a way that alters the sequence, function, and/or expression of an endogenous polynucleotide. In some aspects, the genome of the cell is genetically modified, e.g., using a gene editing tool. In some aspects, the cell is engineered to express a heterologous polypeptide, e.g., a heterologous HLA molecule, a chimeric antigen receptor (CAR), a heterologous T cell receptor (TCR), or any combination thereof.

The term “HLA-binding pocket” or variations thereof, refers to the peptide (e.g., antigen) binding sites of HLA molecules. The HLA-binding pocket is formed by a β-sheet floor comprising eight anti-parallel β-sheets, packed against two anti-parallel α-helices forming a channel. In HLA class I molecules the binding groove is divided into six pockets, A-F, which are defined by specific polymorphic amino acid residues that determine their topography and functionality. The B and F pockets are where the primary anchor residues, the second and last positions of the peptide (P2 and PΩ, respectively), bind to the HLA class I. These class I HLA molecules typically bind peptides 8-11 amino acids in length. Structures of peptide/HLA complexes show that conserved hydrogen bonds are formed between HLA side chains and the peptide backbone of the nine core amino acids within the bound peptide. Additional HLA allele specific interactions are formed between the peptide side chains and structural pockets in the antigen binding pocket.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT), and allogeneic T cell transplantation.

Cells used in an immunotherapy described herein can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety. An immunotherapy can also comprise administering a modified cell to a subject, wherein the modified cell expresses CD3 and a TCR disclosed herein. In some aspects, the modified cell is not a T cell.

A “patient” as used herein includes any human who is afflicted with a cancer (e.g., a lymphoma or a leukemia). The terms “subject” and “patient” are used interchangeably herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex, that specifically binds with a cognate stimulatory ligand present on an antigen present cell. A “stimulatory ligand” is a ligand that when present on an antigen presenting cell (e.g., an aAPC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an MHC Class I molecule loaded with a peptide, an anti-CD3 antibody, a superagonist anti-CD28 antibody, and a superagonist anti-CD2 antibody.

The terms “conditioning” and “pre-conditioning” are used interchangeably herein and indicate preparing a patient in need of a T cell therapy for a suitable condition. Conditioning as used herein includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy. In one aspect, “conditioning” comprises increasing a serum level of one or more cytokines, e.g., interleukin 7 (IL-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof. In another aspect, “conditioning” comprises increasing a serum level of IL-7, IL-15, IP-10, MCP-1, PLGF, CRP, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Various aspects of the invention are described in further detail in the following subsections.

### II. Methods of the Disclosure

Some aspects of the present disclosure are directed to methods of conditioning a subject in need of a therapy comprising modifying an HLA-binding pocket of an HLA molecule expressed on a cell of the subject. Some aspects of the present disclosure are directed to methods of enhancing an immune response in a subject in need thereof, comprising modifying an HLA-binding binding pocket of an HLA molecule expressed on a cell of the subject. Other aspects of the present disclosure are directed to methods of increasing a binding affinity of a peptide to an HLA molecule on a cell comprising modifying a binding pocket of the HLA on a cell. In some aspects, the cell is in a subject in need of a therapy.

Some aspects of the present disclosure are directed to methods of increasing immunogenicity of an antigen in a subject in need of a therapy comprising modifying an HLA-binding pocket of an HLA molecule on a cell in the subject, wherein the HLA molecule is capable of binding the antigen. Some aspects of the present disclosure are directed to methods of increasing an immune response to a therapy in a subject in need thereof comprising modifying an HLA-binding pocket of an HLA molecule in a cell of the subject. In some aspects, the therapy comprises an immunotherapy. Other aspects of the present disclosure are directed to methods of increasing an immune response to an antigen in a subject in need thereof, comprising modifying a binding pocket of an HLA in a cell of the subject.

In some aspects, the modified HLA-binding pocket has increased binding affinity to an antigen. In some aspect, the binding affinity is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, at least about 450%, or at least about 500% relative to the binding affinity of an unmodified HLA molecule.

In some aspects, a cell comprising the HLA molecule comprising the modified HLA-binding pocket has increased surface display of an antigen-HLA complex relative to a cell comprising an unmodified HLA molecule. In some aspects, the surface display of the antigen-HLA complex is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, at least about 450%, or at least about 500% relative to the surface display of the antigen-HLA complex of an unmodified HLA molecule.

In some aspects, the cell is an antigen-presenting cell. In some aspects, the cell is a dendritic cell. In some aspects, the cell is an artificial antigen-presenting cell. In some aspects, the artificial antigen-presenting cell comprises a bead (e.g., a silicate bead, a glass bead, a metal bead, or a combination thereof), a nanovesicle, a microvesicle, an exosome, an endosome, or any combination thereof. In some aspects, the cell is in vivo. In some aspects, the cell is ex vivo. In some aspects, the cell is an allogenic cell. In some aspects, the cell is a donor cell, i.e., a cell obtained from a subject other than the subject that may ultimately receive the cell.

In some aspects, an antigen-HLA complex comprising an HLA molecule having a modified HLA-binding pocket, as disclosed herein, elicits a greater antigen-specific T cell response when contacted with a T cell. In some aspects, the antigen-specific T cell response is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, at least about 450%, or at least about 500% relative to the antigen-specific T cell response when a T cell is contacted with an antigen-HLA complex comprising an unmodified HLA molecule.

In some aspects, an antigen-HLA complex comprising an HLA molecule having a modified HLA-binding pocket, as disclosed herein, increases expansion of tumor-antigen specific T cells. In some aspects, the antigen-specific T cell response is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, at least about 450%, or at least about 500% relative to the expansion of tumor-antigen specific T cells when the T cells are contacted with an antigen-HLA complex comprising an unmodified HLA molecule.

In some aspects, the antigen is a tumor antigen. In some aspects, the antigen is an antigen expressed by a pathogen. In some aspects, the antigen is a viral antigen. In some aspects, the antigen is a bacterial antigen. In some aspects, the antigen is a fungal antigen.

### II.A. HLA Class I Molecules

Described herein are modified HLA molecules with enhanced antigen binding affinity and methods of use thereof. Any HLA molecule can be used in the methods and compositions of the present disclosure. In some aspects, the HLA molecule is an HLA class I molecule. The HLA class I molecule can be any HLA class I molecule. In some aspects, the HLA class I molecule is selected from an HLA-A, HLA-B, HLA-C, HLA-E, HLA-F, HLA-G, HLA-K, or HLA-L allele, or any combination thereof. In some aspects, the HLA class I molecule is selected from an HLA-A, HLA-B, and HLA-C allele. In some aspects, the HLA class I molecule is selected from an HLA-E, HLA-F, and HLA-G allele. In some aspects, the HLA class I molecule is an HLA-A allele. In certain aspects, the HLA class I molecule is an HLA-B allele. In certain aspects, the HLA class I molecule is an HLA-C allele.

Many HLA class I alleles, including many HLA-A, HLA-B, and HLA-C alleles, are known, and any of the known alleles can be used in the present disclosure. An updated list of HLA alleles is available at hla.alleles.org/(last visited on Oct. 7, 2021).

### II.A.1. HLA-A Alleles

In some aspects, the HLA class I molecule is an HLA-A allele. Any HLA-A allele can be used in methods and compositions of the present disclosure. In some aspects, the HLA molecule is an allele selected from HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, HLA-A*34, HLA-A*36, HLA-A*43, HLA-A*66, HLA-A*68, HLA-A*69, HLA-A*74, and HLA-A*80. In some aspects, the HLA molecule is an allele of the HLA-A*24 super family of alleles. In some aspects, the HLA molecule is an HLA-A*24 allele. In some aspects, the HLA molecule is an HLA-A*23 allele. In some aspects, the HLA molecule is an HLA-A*32 allele.

In some aspects, the HLA molecule is an HLA-A allele selected from HLA-A*23:01:01:01, HLA-A*23:01:01:02, HLA-A*23:01:01:03, HLA-A*23:01:01:04, HLA-A*23:01:01:05, HLA-A*23:01:01:06, HLA-A*23:01:01:07, HLA-A*23:01:01:08, HLA-A*23:01:01:09, HLA-A*23:01:01:10, HLA-A*23:01:01:11, HLA-A*23:01:01:12, HLA-A*23:01:01:13, HLA-A*23:01:01:14, HLA-A*23:01:01:15, HLA-A*23:01:01:16, HLA-A*23:01:01:17, HLA-A*23:01:01:18, HLA-A*23:01:01:19, HLA-A*23:01:01:20, HLA-A*23:01:01:21, HLA-A*23:01:01:22, HLA-A*23:01:01:23, HLA-A*23:01:01:24, HLA-A*23:01:01:25, HLA-A*23:01:01:26, HLA-A*23:01:02, HLA-A*23:01:03, HLA-A*23:01:04, HLA-A*23:01:05, HLA-A*23:01:06, HLA-A*23:01:07, HLA-A*23:01:08, HLA-A*23:01:09, HLA-A*23:01:10, HLA-A*23:01:11, HLA-A*23:01:12, HLA-A*23:01:13, HLA-A*23:01:14, HLA-A*23:01:15, HLA-A*23:01:16, HLA-A*23:01:17, HLA-A*23:01:18, HLA-A*23:01:19, HLA-A*23:01:20, HLA-A*23:01:21, HLA-A*23:01:22, HLA-A*23:01:23, HLA-A*23:01:24, HLA-A*23:01:25, HLA-A*23:01:26, HLA-A*23:01:27, HLA-A*23:01:28, HLA-A*23:01:29, HLA-A*23:01:30, HLA-A*23:01:31, HLA-A*23:01:32, HLA-A*23:01:33, HLA-A*23:01:34, HLA-A*23:02, HLA-A*23:03:01, HLA-A*23:03:02:01, HLA-A*23:03:02:02, HLA-A*23:04, HLA-A*23:05, HLA-A*23:06, HLA-A*23:07, HLA-A*23:08, HLA-A*23:09, HLA-A*23:10, HLA-A*23:11, HLA-A*23:12, HLA-A*23:13, HLA-A*23:14:01, HLA-A*23:14:02, HLA-A*23:15, HLA-A*23:16, HLA-A*23:17:01:01, HLA-A*23:17:01:02, HLA-A*23:17:01:03, HLA-A*23:17:02, HLA-A*23:17:03, HLA-A*23:18, HLA-A*23:19, HLA-A*23:20, HLA-A*23:21, HLA-A*23:22, HLA-A*23:23, HLA-A*23:24, HLA-A*23:25, HLA-A*23:26, HLA-A*23:27, HLA-A*23:28, HLA-A*23:29, HLA-A*23:30, HLA-A*23:31, HLA-A*23:32, HLA-A*23:33, HLA-A*23:34, HLA-A*23:35, HLA-A*23:36, HLA-A*23:37:01, HLA-A*23:37:02, HLA-A*23:38, HLA-A*23:39, HLA-A*23:40, HLA-A*23:41, HLA-A*23:42, HLA-A*23:43, HLA-A*23:44, HLA-A*23:45, HLA-A*23:46, HLA-A*23:47, HLA-A*23:48, HLA-A*23:49, HLA-A*23:50, HLA-A*23:51, HLA-A*23:52, HLA-A*23:53, HLA-A*23:54, HLA-A*23:55, HLA-A*23:56, HLA-A*23:57, HLA-A*23:58, HLA-A*23:59, HLA-A*23:60, HLA-A*23:61, HLA-A*23:62, HLA-A*23:63, HLA-A*23:64, HLA-A*23:65, HLA-A*23:66, HLA-A*23:67, HLA-A*23:68, HLA-A*23:70, HLA-A*23:71, HLA-A*23:72, HLA-A*23:73, HLA-A*23:74, HLA-A*23:75, HLA-A*23:76, HLA-A*23:77, HLA-A*23:78, HLA-A*23:79, HLA-A*23:80, HLA-A*23:81, HLA-A*23:82, HLA-A*23:83, HLA-A*23:84, HLA-A*23:85, HLA-A*23:86, HLA-A*23:87, HLA-A*23:88, HLA-A*23:89, HLA-A*23:90, HLA-A*23:91, HLA-A*23:92, HLA-A*23:93, HLA-A*23:94, HLA-A*23:95, HLA-A*23:96, HLA-A*23:97, HLA-A*23:98, HLA-A*23:99, HLA-A*23:100, HLA-A*23:101, HLA-A*23:102, HLA-A*23:103, HLA-A*23:104, HLA-A*23:105, HLA-A*23:106, HLA-A*23:107, HLA-A*23:108, HLA-A*23:109, HLA-A*23:110, HLA-A*23:111, HLA-A*23:112, HLA-A*23:113, HLA-A*23:114, HLA-A*23:115, HLA-A*23:116, and HLA-A*23:117.

In some aspects, the HLA molecule is an HLA-A allele selected from HLA-A*24:02:01:01, HLA-A*24:02:01:02, HLA-A*24:02:01:03, HLA-A*24:02:01:04, HLA-A*24:02:01:05, HLA-A*24:02:01:06, HLA-A*24:02:01:07, HLA-A*24:02:01:08, HLA-A*24:02:01:09, HLA-A*24:02:01:10, HLA-A*24:02:01:11, HLA-A*24:02:01:12, HLA-A*24:02:01:13, HLA-A*24:02:01:14, HLA-A*24:02:01:15, HLA-A*24:02:01:16, HLA-A*24:02:01:17, HLA-A*24:02:01:18, HLA-A*24:02:01:19, HLA-A*24:02:01:20, HLA-A*24:02:01:21, HLA-A*24:02:01:22, HLA-A*24:02:01:23, HLA-A*24:02:01:24, HLA-A*24:02:01:25, HLA-A*24:02:01:26, HLA-A*24:02:01:27, HLA-A*24:02:01:28, HLA-A*24:02:01:29, HLA-A*24:02:01:30, HLA-A*24:02:01:31, HLA-A*24:02:01:32, HLA-A*24:02:01:33, HLA-A*24:02:01:34, HLA-A*24:02:01:35, HLA-A*24:02:01:36, HLA-A*24:02:01:37, HLA-A*24:02:01:38, HLA-A*24:02:01:39, HLA-A*24:02:01:40, HLA-A*24:02:01:41, HLA-A*24:02:01:42, HLA-A*24:02:01:43, HLA-A*24:02:01:44, HLA-A*24:02:01:45, HLA-A*24:02:01:46, HLA-A*24:02:01:47, HLA-A*24:02:01:48, HLA-A*24:02:01:49, HLA-A*24:02:01:50, HLA-A*24:02:01:51, HLA-A*24:02:01:52, HLA-A*24:02:01:53, HLA-A*24:02:01:54, HLA-A*24:02:01:55, HLA-A*24:02:01:56, HLA-A*24:02:01:57, HLA-A*24:02:01:58, HLA-A*24:02:01:59, HLA-A*24:02:01:60, HLA-A*24:02:01:61, HLA-A*24:02:01:62, HLA-A*24:02:01:63, HLA-A*24:02:01:64, HLA-A*24:02:01:65, HLA-A*24:02:01:66, HLA-A*24:02:01:67, HLA-A*24:02:01:68, HLA-A*24:02:01:69, HLA-A*24:02:01:70, HLA-A*24:02:01:71, HLA-A*24:02:01:72, HLA-A*24:02:01:73, HLA-A*24:02:01:74, HLA-A*24:02:01:75, HLA-A*24:02:01:76, HLA-A*24:02:01:77, HLA-A*24:02:01:78, HLA-A*24:02:01:79, HLA-A*24:02:01:80, HLA-A*24:02:01:81, HLA-A*24:02:01:82, HLA-A*24:02:01:83, HLA-A*24:02:01:84, HLA-A*24:02:01:85, HLA-A*24:02:01:86, HLA-A*24:02:01:87, HLA-A*24:02:01:88, HLA-A*24:02:01:89, HLA-A*24:02:01:90, HLA-A*24:02:01:91, HLA-A*24:02:01:92, HLA-A*24:02:01:93, HLA-A*24:02:01:94, HLA-A*24:02:01:95, HLA-A*24:02:01:96, HLA-A*24:02:01:97, HLA-A*24:02:01:98, HLA-A*24:02:01:99, HLA-A*24:02:01:100, HLA-A*24:02:01:101, HLA-A*24:02:01:102, HLA-A*24:02:01:103, HLA-A*24:02:01:104, HLA-A*24:02:01:105, HLA-A*24:02:01:106, HLA-A*24:02:01:107, HLA-A*24:02:01:108, HLA-A*24:02:01:109, HLA-A*24:02:01:110, HLA-A*24:02:02, HLA-A*24:02:03, HLA-A*24:02:04, HLA-A*24:02:05, HLA-A*24:02:06, HLA-A*24:02:07, HLA-A*24:02:08, HLA-A*24:02:09, HLA-A*24:02:10, HLA-A*24:02:11, HLA-A*24:02:12, HLA-A*24:02:13, HLA-A*24:02:14, HLA-A*24:02:15, HLA-A*24:02:16, HLA-A*24:02:17, HLA-A*24:02:18, HLA-A*24:02:19, HLA-A*24:02:20, HLA-A*24:02:21, HLA-A*24:02:22, HLA-A*24:02:23, HLA-A*24:02:24, HLA-A*24:02:25, HLA-A*24:02:26, HLA-A*24:02:27, HLA-A*24:02:28, HLA-A*24:02:29, HLA-A*24:02:30, HLA-A*24:02:31, HLA-A*24:02:32, HLA-A*24:02:33, HLA-A*24:02:34, HLA-A*24:02:35, HLA-A*24:02:36, HLA-A*24:02:37, HLA-A*24:02:38, HLA-A*24:02:39, HLA-A*24:02:40:01, HLA-A*24:02:40:02, HLA-A*24:02:41, HLA-A*24:02:42, HLA-A*24:02:43, HLA-A*24:02:44, HLA-A*24:02:45, HLA-A*24:02:46, HLA-A*24:02:47, HLA-A*24:02:48, HLA-A*24:02:49, HLA-A*24:02:50, HLA-A*24:02:51, HLA-A*24:02:52, HLA-A*24:02:53, HLA-A*24:02:54, HLA-A*24:02:55, HLA-A*24:02:56, HLA-A*24:02:57, HLA-A*24:02:58, HLA-A*24:02:59, HLA-A*24:02:60, HLA-A*24:02:61, HLA-A*24:02:62, HLA-A*24:02:63, HLA-A*24:02:64, A*24:02:69, HLA-A*24:02:70, HLA-A*24:02:71, HLA-A*24:02:72, HLA-A*24:02:73, HLA-A*24:02:74, HLA-A*24:02:75, HLA-A*24:02:76, HLA-A*24:02:77, HLA-A*24:02:78, HLA-A*24:02:79, HLA-A*24:02:80, HLA-A*24:02:81, HLA-A*24:02:82, HLA-A*24:02:83, HLA-A*24:02:84, HLA-A*24:02:85, HLA-A*24:02:86, HLA-A*24:02:87, HLA-A*24:02:88, HLA-A*24:02:89, HLA-A*24:02:90, HLA-A*24:02:91, HLA-A*24:02:92, HLA-A*24:02:93, HLA-A*24:02:94, HLA-A*24:02:95, HLA-A*24:02:96, HLA-A*24:02:97, HLA-A*24:02:98, HLA-A*24:02:99, HLA-A*24:02:100, HLA-A*24:02:101, HLA-A*24:02:102:01, HLA-A*24:02:102:02, HLA-A*24:02:103, HLA-A*24:02:104, HLA-A*24:02:105, HLA-A*24:02:106, HLA-A*24:02:107, HLA-A*24:02:108, HLA-A*24:02:109, HLA-A*24:02:110, HLA-A*24:02:111, HLA-A*24:02:112, HLA-A*24:02:113, HLA-A*24:02:114, HLA-A*24:02:115:01, HLA-A*24:02:115:02, HLA-A*24:02:116, HLA-A*24:02:117, HLA-A*24:02:118, HLA-A*24:02:119, HLA-A*24:02:120, HLA-A*24:02:121, HLA-A*24:02:122, HLA-A*24:02:123, HLA-A*24:02:124, HLA-A*24:02:125, HLA-A*24:02:126, HLA-A*24:02:127, HLA-A*24:02:128, HLA-A*24:02:129, HLA-A*24:02:130, HLA-A*24:02:131, HLA-A*24:02:132, HLA-A*24:02:133, HLA-A*24:02:134, HLA-A*24:02:135, HLA-A*24:02:136, HLA-A*24:02:137, HLA-A*24:02:138, HLA-A*24:02:139, HLA-A*24:02:140, HLA-A*24:02:141, HLA-A*24:02:142, HLA-A*24:02:143, HLA-A*24:02:144, HLA-A*24:02:145, HLA-A*24:02:146, HLA-A*24:02:147, HLA-A*24:02:148, HLA-A*24:02:149, HLA-A*24:03:01:01, HLA-A*24:03:01:02, HLA-A*24:03:01:03, HLA-A*24:03:02, HLA-A*24:03:03, HLA-A*24:03:04, HLA-A*24:04, HLA-A*24:05:01, HLA-A*24:05:02, HLA-A*24:06, HLA-A*24:07:01:01, HLA-A*24:07:01:02, HLA-A*24:07:01:03, HLA-A*24:07:02, HLA-A*24:07:03, HLA-A*24:07:04, HLA-A*24:08, HLA-A*24:09, HLA-A*24:10:01:01, HLA-A*24:10:01:02, HLA-A*24:10:02, HLA-A*24:11, HLA-A*24:13:01, HLA-A*24:13:02, HLA-A*24:14:01:01, HLA-A*24:14:01:02, HLA-A*24:14:01:03, HLA-A*24:14:01:04, HLA-A*24:15, HLA-A*24:17:01:01, HLA-A*24:17:01:02, HLA-A*24:18, HLA-A*24:19, HLA-A*24:20:01:01, HLA-A*24:20:01:02, HLA-A*24:20:02, HLA-A*24:21:01, HLA-A*24:21:02, HLA-A*24:21:03, HLA-A*24:22, HLA-A*24:23, HLA-A*24:24, HLA-A*24:25, HLA-A*24:26, HLA-A*24:27, HLA-A*24:28, HLA-A*24:29, HLA-A*24:30, HLA-A*24:31, HLA-A*24:32, HLA-A*24:33, HLA-A*24:34, HLA-A*24:35, HLA-A*24:36, HLA-A*24:37, HLA-A*24:38, HLA-A*24:39, HLA-HLA-A*24:46, HLA-A*24:47, HLA-A*24:48, HLA-A*24:49, HLA-A*24:50, HLA-A*24:51, HLA-A*24:52, HLA-A*24:53, HLA-A*24:54, HLA-A*24:55, HLA-A*24:56:01, HLA-A*24:56:02, HLA-A*24:57, HLA-A*24:58, HLA-A*24:59, HLA-A*24:60, HLA-A*24:61, HLA-A*24:62, HLA-A*24:63, HLA-A*24:64, HLA-A*24:66, HLA-A*24:67, HLA-A*24:68, HLA-A*24:69, HLA-A*24:70, HLA-A*24:71, HLA-A*24:72, HLA-A*24:73, HLA-A*24:74:01, HLA-A*24:74:02, HLA-A*24:75, HLA-A*24:76, HLA-A*24:77, HLA-A*24:78, HLA-A*24:79, HLA-A*24:80, HLA-A*24:81, HLA-A*24:82, HLA-A*24:83, HLA-A*24:84, HLA-A*24:85, HLA-A*24:86, HLA-A*24:87, HLA-A*24:88, HLA-A*24:89, HLA-A*24:90:01, HLA-A*24:90:02, HLA-A*24:91, HLA-A*24:92, HLA-A*24:93, HLA-A*24:94, HLA-A*24:95, HLA-A*24:96, HLA-A*24:97, HLA-A*24:98, HLA-A*24:99, HLA-A*24:100, HLA-A*24:101, HLA-A*24:102, HLA-A*24:103, HLA-A*24:104, HLA-A*24:105, HLA-A*24:106, HLA-A*24:107, HLA-A*24:108, HLA-A*24:109, HLA-A*24:110, HLA-A*24:111, HLA-A*24:112, HLA-A*24:113, HLA-A*24:114, HLA-A*24:115, HLA-A*24:116, HLA-A*24:117, HLA-A*24:118, HLA-A*24:119, HLA-A*24:120, HLA-A*24:121, HLA-A*24:122, HLA-A*24:123, HLA-A*24:124, HLA-A*24:125, HLA-A*24:126, HLA-A*24:127, HLA-A*24:128, HLA-A*24:129, HLA-A*24:130, HLA-A*24:131, HLA-A*24:132, HLA-A*24:133, HLA-A*24:134, HLA-A*24:135:01, HLA-A*24:135:02, HLA-A*24:136, HLA-A*24:137, HLA-A*24:138, HLA-A*24:139, HLA-A*24:140, HLA-A*24:141, HLA-A*24:142:01, HLA-A*24:142:02, HLA-A*24:143, HLA-A*24:144, HLA-A*24:145, HLA-A*24:146, HLA-A*24:147, HLA-A*24:148, HLA-A*24:149, HLA-A*24:150, HLA-A*24:151, HLA-A*24:152, HLA-A*24:153, HLA-A*24:154, HLA-A*24:155, HLA-A*24:156, HLA-A*24:157, HLA-A*24:158, HLA-A*24:159, HLA-A*24:160, HLA-A*24:161, HLA-A*24:162, HLA-A*24:163, HLA-A*24:164, HLA-A*24:165, HLA-A*24:166, HLA-A*24:167, HLA-A*24:168, HLA-A*24:169, HLA-A*24:170, HLA-A*24:171, HLA-A*24:172:01, HLA-A*24:172:02, HLA-A*24:173, HLA-A*24:174, HLA-A*24:175, HLA-A*24:176, HLA-A*24:177, HLA-A*24:178, HLA-A*24:179, HLA-A*24:180, HLA-A*24:181, HLA-A*24:182, HLA-A*24:183, HLA-A*24:184, HLA-A*24:185, HLA-A*24:186, HLA-A*24:187, HLA-A*24:188, HLA-A*24:189, HLA-A*24:190, HLA-A*24:191, HLA-A*24:192, HLA-A*24:193, HLA-A*24:194, HLA-A*24:195, HLA-A*24:196, HLA-A*24:197, HLA-A*24:198, HLA-A*24:199, HLA-A*24:200, HLA-A*24:201, HLA-A*24:202, HLA-A*24:203, HLA-HLA-A*24:208:01, HLA-A*24:208:02:01, HLA-A*24:208:02:02, HLA-A*24:209, HLA-A*24:210, HLA-A*24:212, HLA-A*24:213, HLA-A*24:214, HLA-A*24:215, HLA-A*24:216, HLA-A*24:217, HLA-A*24:218, HLA-A*24:219, HLA-A*24:220, HLA-A*24:221, HLA-A*24:222, HLA-A*24:223, HLA-A*24:224, HLA-A*24:225:01, HLA-A*24:225:02, HLA-A*24:226:01, HLA-A*24:226:02, HLA-A*24:227, HLA-A*24:228, HLA-A*24:229, HLA-A*24:230, HLA-A*24:231, HLA-A*24:232, HLA-A*24:233, HLA-A*24:234, HLA-A*24:235, HLA-A*24:236, HLA-A*24:237, HLA-A*24:238, HLA-A*24:239, HLA-A*24:240, HLA-A*24:241, HLA-A*24:242, HLA-A*24:243, HLA-A*24:244, HLA-A*24:245, HLA-A*24:246, HLA-A*24:247, HLA-A*24:248, HLA-A*24:249, HLA-A*24:250, HLA-A*24:251, HLA-A*24:252, HLA-A*24:253, HLA-A*24:254, HLA-A*24:255, HLA-A*24:256, HLA-A*24:257, HLA-A*24:258, HLA-A*24:259, HLA-A*24:260, HLA-A*24:261, HLA-A*24:262, HLA-A*24:263, HLA-A*24:264, HLA-A*24:265, HLA-A*24:266, HLA-A*24:267, HLA-A*24:268, HLA-A*24:269, HLA-A*24:270, HLA-A*24:271, HLA-A*24:272, HLA-A*24:273, HLA-A*24:274, HLA-A*24:275, HLA-A*24:276, HLA-A*24:277, HLA-A*24:278, HLA-A*24:279, HLA-A*24:280, HLA-A*24:281, HLA-A*24:282, HLA-A*24:283, HLA-A*24:284, HLA-A*24:285, HLA-A*24:286, HLA-A*24:287, HLA-A*24:288, HLA-A*24:289, HLA-A*24:290, HLA-A*24:291, HLA-A*24:292, HLA-A*24:293, HLA-A*24:294, HLA-A*24:295, HLA-A*24:296, HLA-A*24:297, HLA-A*24:298, HLA-A*24:299, HLA-A*24:300, HLA-A*24:301, HLA-A*24:302, HLA-A*24:303, HLA-A*24:304, HLA-A*24:305, HLA-A*24:306, HLA-A*24:307, HLA-A*24:308, HLA-A*24:309, HLA-A*24:310:01, HLA-A*24:310:02, HLA-A*24:311, HLA-A*24:312, HLA-A*24:313:01, HLA-A*24:313:02, HLA-A*24:314, HLA-A*24:315, HLA-A*24:316, HLA-A*24:317, HLA-A*24:318, HLA-A*24:319, HLA-A*24:320, HLA-A*24:321, HLA-A*24:322, HLA-A*24:323, HLA-A*24:324, HLA-A*24:325, HLA-A*24:326, HLA-A*24:327, HLA-A*24:328, HLA-A*24:329, HLA-A*24:330, HLA-A*24:331, HLA-A*24:332, HLA-A*24:333, HLA-A*24:334, HLA-A*24:335, HLA-A*24:336, HLA-A*24:337, HLA-A*24:338, HLA-A*24:339, HLA-A*24:340, HLA-A*24:341, HLA-A*24:342, HLA-A*24:343, HLA-A*24:344, HLA-A*24:345, HLA-A*24:346, HLA-A*24:347:01, HLA-A*24:347:02, HLA-A*24:348, HLA-A*24:349, HLA-A*24:350, HLA-A*24:351, HLA-A*24:352, HLA-A*24:353, HLA-A*24:354, HLA-A*24:355, HLA-A*24:356, HLA-A*24:357, HLA-A*24:358, HLA-A*24:359, HLA-A*24:360, HLA-A*24:366, HLA-A*24:367, HLA-A*24:368, HLA-A*24:369, HLA-A*24:370, HLA-A*24:371, HLA-A*24:372, HLA-A*24:373, HLA-A*24:374, HLA-A*24:375, HLA-A*24:376, HLA-A*24:377, HLA-A*24:378, HLA-A*24:379, HLA-A*24:380, HLA-A*24:381, HLA-A*24:382, HLA-A*24:383, HLA-A*24:384, HLA-A*24:385, HLA-A*24:386, HLA-A*24:387, HLA-A*24:388, HLA-A*24:389, HLA-A*24:390, HLA-A*24:391, HLA-A*24:392, HLA-A*24:393, HLA-A*24:394, HLA-A*24:395, HLA-A*24:396, HLA-A*24:397, HLA-A*24:398, HLA-A*24:399, HLA-A*24:400, HLA-A*24:401, HLA-A*24:402, HLA-A*24:403, HLA-A*24:404, HLA-A*24:405, HLA-A*24:406, HLA-A*24:407, HLA-A*24:408, HLA-A*24:409, HLA-A*24:410, HLA-A*24:411, HLA-A*24:412, HLA-A*24:413, HLA-A*24:414, HLA-A*24:415, HLA-A*24:416, HLA-A*24:417, HLA-A*24:418, HLA-A*24:419, HLA-A*24:420, HLA-A*24:421, HLA-A*24:422, HLA-A*24:423, HLA-A*24:424, HLA-A*24:425, HLA-A*24:426, HLA-A*24:427, HLA-A*24:428, HLA-A*24:429, HLA-A*24:430, HLA-A*24:431:01, HLA-A*24:431:02, HLA-A*24:432, HLA-A*24:433, HLA-A*24:434, HLA-A*24:435, HLA-A*24:436, HLA-A*24:437, HLA-A*24:438, HLA-A*24:439, HLA-A*24:440, HLA-A*24:441, HLA-A*24:442, HLA-A*24:443, HLA-A*24:444, HLA-A*24:445, HLA-A*24:446, HLA-A*24:447, HLA-A*24:448, HLA-A*24:449, HLA-A*24:450, HLA-A*24:451, HLA-A*24:452, HLA-A*24:453, HLA-A*24:454, HLA-A*24:455, HLA-A*24:456, HLA-A*24:457, HLA-A*24:458, HLA-A*24:459, HLA-A*24:460:01:01, HLA-A*24:460:01:02, HLA-A*24:461, HLA-A*24:462, HLA-A*24:463, HLA-A*24:464, HLA-A*24:465, HLA-A*24:466, HLA-A*24:467, HLA-A*24:468, HLA-A*24:469, HLA-A*24:470, HLA-A*24:472, HLA-A*24:473, HLA-A*24:474, HLA-A*24:475, HLA-A*24:476, HLA-A*24:477, HLA-A*24:478, HLA-A*24:479, HLA-A*24:480, HLA-A*24:481, HLA-A*24:482, HLA-A*24:483, HLA-A*24:484, HLA-A*24:485, HLA-A*24:486, HLA-A*24:487, HLA-A*24:488, HLA-A*24:489, HLA-A*24:490, HLA-A*24:491, HLA-A*24:492, HLA-A*24:493, HLA-A*24:494, HLA-A*24:495, HLA-A*24:496, HLA-A*24:497, HLA-A*24:498, HLA-A*24:499, HLA-A*24:500, HLA-A*24:501, HLA-A*24:502, HLA-A*24:503, HLA-A*24:504, HLA-A*24:505, HLA-A*24:506, HLA-A*24:507, HLA-A*24:508, HLA-A*24:509, HLA-A*24:510, HLA-A*24:511, HLA-A*24:512, HLA-A*24:513, HLA-A*24:514, HLA-A*24:515, HLA-A*24:516, HLA-A*24:517, HLA-A*24:518, HLA-A*24:519, HLA-A*24:520, HLA-A*24:521, HLA-A*24:522, HLA-A*24:523, HLA-A*24:529, HLA-A*24:530, HLA-A*24:531, HLA-A*24:532, HLA-A*24:533, HLA-A*24:534, HLA-A*24:535, HLA-A*24:536, HLA-A*24:537, HLA-A*24:538, HLA-A*24:539, HLA-A*24:540, HLA-A*24:541, HLA-A*24:542, HLA-A*24:543, HLA-A*24:544, HLA-A*24:545, HLA-A*24:546, HLA-A*24:547, HLA-A*24:548, HLA-A*24:549, HLA-A*24:550, and HLA-A*24:551. In some aspects, the HLA molecule is an HLA-A*24:02 allele.

In some aspects, the HLA molecule is an HLA-A allele selected from HLA-A*25:01:01:01, HLA-A*25:01:01:02, HLA-A*25:01:01:03, HLA-A*25:01:01:04, HLA-A*25:01:01:05, HLA-A*25:01:01:06, HLA-A*25:01:01:07, HLA-A*25:01:02, HLA-A*25:01:03, HLA-A*25:01:04, HLA-A*25:01:05, HLA-A*25:01:06, HLA-A*25:01:07, HLA-A*25:01:08, HLA-A*25:01:09, HLA-A*25:01:10, HLA-A*25:01:11, HLA-A*25:01:12, HLA-A*25:01:13, HLA-A*25:01:14, HLA-A*25:01:15, HLA-A*25:01:16, HLA-A*25:01:17, HLA-A*25:01:18, HLA-A*25:01:19, HLA-A*25:01:20, HLA-A*25:01:21, HLA-A*25:02, HLA-A*25:03, HLA-A*25:04, HLA-A*25:05, HLA-A*25:06, HLA-A*25:07, HLA-A*25:08, HLA-A*25:09, HLA-A*25:10, HLA-A*25:11, HLA-A*25:12, HLA-A*25:13, HLA-A*25:14, HLA-A*25:15, HLA-A*25:16, HLA-A*25:17, HLA-A*25:18, HLA-A*25:19:01, HLA-A*25:19:02, HLA-A*25:20, HLA-A*25:21, HLA-A*25:22, HLA-A*25:23, HLA-A*25:24, HLA-A*25:25, HLA-A*25:26, HLA-A*25:27:01, HLA-A*25:27:02, HLA-A*25:28, HLA-A*25:29, HLA-A*25:30, HLA-A*25:31, HLA-A*25:32, HLA-A*25:33, HLA-A*25:34, HLA-A*25:35, HLA-A*25:36, HLA-A*25:37, HLA-A*25:38, HLA-A*25:39, HLA-A*25:40, HLA-A*25:41, HLA-A*25:42, HLA-A*25:43, HLA-A*25:44, HLA-A*25:45, HLA-A*25:46, HLA-A*25:47, HLA-A*25:48, HLA-A*25:49, HLA-A*25:50, HLA-A*25:51, HLA-A*25:52, HLA-A*25:53, HLA-A*25:54, HLA-A*25:55, HLA-A*25:56, HLA-A*25:57, HLA-A*25:58, HLA-A*25:59, HLA-A*25:60, HLA-A*25:61, HLA-A*25:62, HLA-A*25:63, HLA-A*25:64, HLA-A*25:65, HLA-A*25:66, HLA-A*25:67, HLA-A*25:68, HLA-A*25:69, HLA-A*25:70, HLA-A*25:71, HLA-A*25:72, HLA-A*25:73, HLA-A*25:74, HLA-A*25:75, and HLA-A*25:76.

In some aspects, the HLA molecule is an HLA-A allele selected from HLA-A*31:01:02:01, HLA-A*31:01:02:02, HLA-A*31:01:02:03, HLA-A*31:01:02:04, HLA-A*31:01:02:05, HLA-A*31:01:02:06, HLA-A*31:01:02:07, HLA-A*31:01:02:08, HLA-A*31:01:02:09, HLA-A*31:01:02:10, HLA-A*31:01:02:11, HLA-A*31:01:02:12, HLA-A*31:01:02:13, HLA-A*31:01:02:14, HLA-A*31:01:02:15, HLA-A*31:01:02:16, HLA-A*31:01:02:17, HLA-A*31:01:02:18, HLA-A*31:01:02:19, HLA-A*31:01:02:20, HLA-A*31:01:02:21, HLA-A*31:01:02:22, HLA-A*31:01:02:23, HLA-A*31:01:02:24, HLA-A*31:01:02:25, HLA-A*31:01:02:26, HLA-A*31:01:02:27, HLA-A*31:01:02:28, HLA-A*31:01:02:29, HLA-A*31:01:02:30, HLA-A*31:01:02:31, HLA-A*31:01:02:32, HLA-A*31:01:02:33, HLA-A*31:01:02:34, HLA-A*31:01:02:35, HLA-A*31:01:02:36, HLA-A*31:01:02:37, HLA-A*31:01:02:38, HLA-A*31:01:02:39, HLA-A*31:01:02:40, HLA-A*31:01:02:41, HLA-A*31:01:03, HLA-A*31:01:04, HLA-A*31:01:05, HLA-A*31:01:06, HLA-A*31:01:07, HLA-A*31:01:08, HLA-A*31:01:09, HLA-A*31:01:10, HLA-A*31:01:11, HLA-A*31:01:12, HLA-A*31:01:13, HLA-A*31:01:14, HLA-A*31:01:15, HLA-A*31:01:16, HLA-A*31:01:17, HLA-A*31:01:18, HLA-A*31:01:19, HLA-A*31:01:20, HLA-A*31:01:21, HLA-A*31:01:22, HLA-A*31:01:23, HLA-A*31:01:24, HLA-A*31:01:25, HLA-A*31:01:26, HLA-A*31:01:27, HLA-A*31:01:28, HLA-A*31:01:29, HLA-A*31:01:30, HLA-A*31:01:31, HLA-A*31:01:32, HLA-A*31:01:33, HLA-A*31:01:34, HLA-A*31:01:35, HLA-A*31:01:36, HLA-A*31:01:37, HLA-A*31:01:38, HLA-A*31:01:39, HLA-A*31:01:40, HLA-A*31:01:41, HLA-A*31:01:42, HLA-A*31:01:43, HLA-A*31:01:44, HLA-A*31:01:45, HLA-A*31:01:46, HLA-A*31:01:47, HLA-A*31:02:01, HLA-A*31:02:02, HLA-A*31:03, HLA-A*31:04:01:01, HLA-A*31:04:01:02, HLA-A*31:04:02, HLA-A*31:05, HLA-A*31:06, HLA-A*31:07, HLA-A*31:08, HLA-A*31:09, HLA-A*31:10, HLA-A*31:11, HLA-A*31:12, HLA-A*31:13, HLA-A*31:14, HLA-A*31:15, HLA-A*31:16, HLA-A*31:17, HLA-A*31:18, HLA-A*31:19, HLA-A*31:20, HLA-A*31:21, HLA-A*31:22, HLA-A*31:23, HLA-A*31:24, HLA-A*31:25, HLA-A*31:26, HLA-A*31:27, HLA-A*31:28, HLA-A*31:29, HLA-A*31:30, HLA-A*31:31, HLA-A*31:32, HLA-A*31:33, HLA-A*31:34, HLA-A*31:35, HLA-A*31:36, HLA-A*31:37, HLA-A*31:38, HLA-A*31:39, HLA-A*31:40, HLA-A*31:41, HLA-A*31:42, HLA-A*31:43, HLA-A*31:44, HLA-A*31:45, HLA-A*31:46, HLA-A*31:47, HLA-A*31:48, HLA-A*31:49, HLA-A*31:50, HLA-A*31:51, HLA-A*31:52, HLA-A*31:53, HLA-A*31:54, HLA-A*31:55, HLA-A*31:56, HLA-A*31:57, HLA-A*31:58, HLA-A*31:59, HLA-A*31:60, HLA-A*31:61, HLA-A*31:62, HLA-A*31:63, HLA-A*31:64, HLA-A*31:65, HLA-A*31:66, HLA-A*31:67, HLA-A*31:68, HLA-A*31:69, HLA-A*31:70, HLA-A*31:71, HLA-A*31:72, HLA-A*31:73, HLA-A*31:74, HLA-A*31:75, HLA-A*31:76, HLA-A*31:77, HLA-A*31:78, HLA-A*31:79, HLA-A*31:80, HLA-A*31:81, HLA-A*31:82, HLA-A*31:83, HLA-A*31:84, HLA-A*31:85, HLA-A*31:86, HLA-A*31:87, HLA-A*31:88, HLA-A*31:89, HLA-A*31:90, HLA-A*31:91, HLA-A*31:92, HLA-A*31:93, HLA-A*31:94, HLA-A*31:95, HLA-A*31:96, HLA-A*31:97, HLA-A*31:98, HLA-A*31:99, HLA-A*31:100, HLA-A*31:101, HLA-A*31:102, HLA-A*31:103, HLA-A*31:104, HLA-A*31:105, HLA-A*31:106, HLA-A*31:107, HLA-A*31:108, HLA-A*31:109, HLA-A*31:110, HLA-A*31:111, HLA-A*31:112, HLA-A*31:113, HLA-A*31:114, HLA-A*31:115, HLA-A*31:116, HLA-A*31:117, HLA-A*31:118, HLA-A*31:119, HLA-A*31:120, HLA-A*31:121, HLA-A*31:122, HLA-A*31:123, HLA-A*31:124, HLA-A*31:125, HLA-A*31:126, HLA-A*31:127, HLA-A*31:128, HLA-A*31:129, HLA-A*31:130, HLA-A*31:131, HLA-A*31:132, HLA-A*31:133, HLA-A*31:134, HLA-A*31:135, HLA-A*31:136, HLA-A*31:137, HLA-A*31:138, HLA-A*31:139, HLA-A*31:140, HLA-A*31:141, HLA-A*31:142, HLA-A*31:143, HLA-A*31:144, HLA-A*31:145, HLA-A*31:146, HLA-A*31:147, HLA-A*31:148, HLA-A*31:149, HLA-A*31:150, HLA-A*31:151, HLA-A*31:152, HLA-A*31:153, HLA-A*31:154, HLA-A*31:155, HLA-A*31:156, HLA-A*31:157, HLA-A*31:158, HLA-A*31:159, HLA-A*31:160, HLA-A*31:161, HLA-A*31:162, HLA-A*31:163, HLA-A*31:164, HLA-A*31:165, HLA-A*31:166, HLA-A*31:167, HLA-A*31:168, HLA-A*31:169, HLA-A*31:170, HLA-A*31:171, HLA-A*31:172, HLA-A*31:173, HLA-A*31:174, HLA-A*31:175, HLA-A*31:176, HLA-A*31:177, HLA-A*31:178, HLA-A*31:179, HLA-A*31:180, HLA-A*31:181, HLA-A*31:182, HLA-A*31:183, HLA-A*31:184, HLA-A*31:185, HLA-A*31:186, HLA-A*31:187, HLA-A*31:188, HLA-A*31:189, HLA-A*31:190, HLA-A*31:191, HLA-A*31:192, HLA-A*31:193, HLA-A*31:194, HLA-A*31:195, HLA-A*31:196, HLA-A*31:197, and HLA-A*31:198.

In some aspects, the HLA molecule is an HLA-A allele selected from HLA-A* 32:01:01:01, HLA-A* 32:01:01:02, HLA-A* 32:01:01:03, HLA-A* 32:01:01:04, HLA-A* 32:01:01:05, HLA-A* 32:01:01:06, HLA-A* 32:01:01:07, HLA-A* 32:01:01:08, HLA-A* 32:01:01:09, HLA-A* 32:01:01:10, HLA-A* 32:01:01:11, HLA-A* 32:01:01:12, HLA-A* 32:01:01:13, HLA-A* 32:01:01:14, HLA-A* 32:01:01:15, HLA-A* 32:01:01:16, HLA-A* 32:01:01:17, HLA-A* 32:01:01:18, HLA-A* 32:01:01:19, HLA-A* 32:01:01:20, HLA-A* 32:01:01:21, HLA-A* 32:01:01:22, HLA-A* 32:01:01:23, HLA-A* 32:01:01:24, HLA-A* 32:01:01:25, HLA-A* 32:01:01:26, HLA-A* 32:01:01:27, HLA-A* 32:01:01:28, HLA-A* 32:01:01:29, HLA-A* 32:01:01:30, HLA-A* 32:01:02, HLA-A* 32:01:03, HLA-A* 32:01:04, HLA-A* 32:01:05, HLA-A* 32:01:06, HLA-A* 32:01:07, HLA-A* 32:01:08, HLA-A* 32:01:09, HLA-A* 32:01:10, HLA-A* 32:01:11, HLA-A* 32:01:12, HLA-A* 32:01:13, HLA-A* 32:01:14, HLA-A* 32:01:15, HLA-A* 32:01:16, HLA-A* 32:01:17, HLA-A* 32:01:18, HLA-A* 32:01:19, HLA-A* 32:01:20, HLA-A* 32:01:21, HLA-A* 32:01:22, HLA-A* 32:01:23, HLA-A* 32:01:24, HLA-A* 32:01:25, HLA-A* 32:01:26, HLA-A* 32:01:27, HLA-A* 32:01:28, HLA-A* 32:01:29, HLA-A* 32:01:30, HLA-A* 32:01:31, HLA-A* 32:01:32, HLA-A* 32:01:33, HLA-A* 32:01:34, HLA-A* 32:01:35, HLA-A* 32:01:36, HLA-A* 32:01:37, HLA-A* 32:01:38, HLA-A* 32:01:39, HLA-A* 32:01:40, HLA-A* 32:01:41, HLA-A* 32:01:42, HLA-A* 32:01:43, HLA-A* 32:01:44, HLA-A* 32:01:45, HLA-A* 32:01:46, HLA-A* 32:01:47, HLA-A* 32:02, HLA-A* 32:03:01:01, HLA-A* 32:03:01:02, HLA-A* 32:04, HLA-A* 32:05, HLA-A* 32:06, HLA-A* 32:07, HLA-A* 32:08, HLA-A* 32:09, HLA-A* 32:10, HLA-A* 32:11, HLA-A* 32:12, HLA-A* 32:13, HLA-A* 32:14, HLA-A* 32:15, HLA-A* 32:16, HLA-A* 32:17, HLA-A* 32:18, HLA-A* 32:19, HLA-A* 32:20, HLA-A* 32:21, HLA-A* 32:22, HLA-A* 32:23, HLA-A* 32:24, HLA-A* 32:25, HLA-A* 32:26:01, HLA-A* 32:26:02, HLA-A* 32:27, HLA-A* 32:28, HLA-A* 32:29, HLA-A* 32:30:01, HLA-A* 32:30:02, HLA-A* 32:31, HLA-A* 32:32, HLA-A* 32:33:01, HLA-A* 32:33:02, HLA-A* 32:33:03, HLA-A* 32:34, HLA-A* 32:35, HLA-A* 32:36, HLA-A* 32:37, HLA-A* 32:38, HLA-A* 32:39, HLA-A* 32:40, HLA-A* 32:41, HLA-A* 32:42, HLA-A* 32:43:01, HLA-A* 32:43:02, HLA-A* 32:44, HLA-A* 32:45, HLA-A* 32:46:01, HLA-A* 32:46:02, HLA-A* 32:47, HLA-A* 32:48, HLA-A* 32:49, HLA-A* 32:50, HLA-A* 32:51, HLA-A* 32:52, HLA-A* 32:53, HLA-A* 32:54, HLA-A* 32:55:01, HLA-A* 32:55:02, HLA-A* 32:55:03, HLA-A* 32:56, HLA-A* 32:57, HLA-A* 32:58, HLA-A* 32:59, HLA-A* 32:60, HLA-A* 32:61, HLA-A* 32:62, HLA-A* 32:63, HLA-A* 32:64, HLA-A* 32:65, HLA-A* 32:66, HLA-A* 32:67, HLA-A* 32:68, HLA-A* 32:69, HLA-A* 32:70, HLA-A* 32:71, HLA-A* 32:72, HLA-A* 32:73, HLA-A* 32:74, HLA-A* 32:75, HLA-A* 32:76, HLA-A* 32:77, HLA-A* 32:78, HLA-A* 32:79, HLA-A* 32:80, HLA-A* 32:81, HLA-A* 32:82, HLA-A* 32:83, HLA-A* 32:84, HLA-A* 32:85, HLA-A* 32:86, HLA-A* 32:87, HLA-A* 32:88, HLA-A* 32:89, HLA-A* 32:90, HLA-A* 32:91, HLA-A* 32:92, HLA-A* 32:93, HLA-A* 32:94, HLA-A* 32:95, HLA-A* 32:96, HLA-A* 32:97, HLA-A* 32:98, HLA-A* 32:99, HLA-A* 32:100, HLA-A* 32:101, HLA-A* 32:102, HLA-A* 32:103, HLA-A* 32:104, HLA-A* 32:105, HLA-A* 32:106:01:01, HLA-A* 32:106:01:02, HLA-A* 32:107, HLA-A* 32:108, HLA-A* 32:109, HLA-A* 32:110, HLA-A* 32:111, HLA-A* 32:112, HLA-A* 32:113, HLA-A* 32:114, HLA-A* 32:115, HLA-A* 32:116, HLA-A* 32:117, HLA-A* 32:118, HLA-A* 32:119, HLA-A* 32:120, HLA-A* 32:121, HLA-A* 32:122, HLA-A* 32:123, HLA-A* 32:124, HLA-A* 32:125, HLA-A* 32:126, HLA-A* 32:127, HLA-A* 32:128, HLA-A* 32:129, HLA-A* 32:130, HLA-A* 32:131, HLA-A* 32:132, HLA-A* 32:133, HLA-A* 32:134, HLA-A* 32:135, HLA-A* 32:136, HLA-A* 32:137, HLA-A* 32:138, HLA-A* 32:139, HLA-A* 32:140, HLA-A* 32:141, HLA-A* 32:142, HLA-A* 32:143, HLA-A* 32:144, HLA-A* 32:145, HLA-A* 32:146, HLA-A* 32:147, HLA-A* 32:148, HLA-A* 32:149, HLA-A* 32:150, HLA-A* 32:151, HLA-A* 32:152, HLA-A* 32:153, HLA-A* 32:154, HLA-A* 32:155, and HLA-A* 32:156.

In some aspects, a HLA molecule of the disclosure comprises a modified HLA-binding pocket, wherein the HLA molecule is an HLA-A allele. In some aspects, the modified HLA-binding pocket is the F pocket of the HLA molecule. In some aspects, a HLA molecule of the disclosure comprises a modified HLA-binding pocket, wherein the HLA molecule is an allele within the HLA-A*24 superfamily of alleles. In some aspects, a HLA molecule of the disclosure comprises a modified HLA-binding pocket, wherein the HLA molecule is an HLA-A*24 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-A*24:02 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-A*24 allele, and wherein the modified HLA-binding pocket comprises an amino acid substitution at amino acid residue 81, corresponding to the amino acid sequence set forth in SEQ ID NO: 1. In some aspects, a HLA molecule of the disclosure comprises a modified HLA-binding pocket, wherein the HLA molecule is an HLA-A*24:02 allele, and wherein the modified HLA-binding pocket comprises an amino acid substitution at amino acid residue 81, corresponding to the amino acid sequence set forth in SEQ ID NO: 1.

TABLE 2
HLA-A*24:02 amino acid sequence.

GSHSMRYFSTSVSRPGRGEPRFIAVGYVDDTQFVRFDSDAASQRMEPRAP
WIEQEGPEYWDEETGKVKAHSQTDRENLRIALRYYNQSEAGSHTLQMMFG
CDVGSDGRFLRGYHQYAYDGKDYIALKEDLRSWTAADMAAQITKRKWEAA
HVAEQQRAYLEGTCVDGLRRYLENGKETLQRTDPPKTHMTHHPISDHEAT
LRCWALGFYPAEITLTWQRDGEDQTQDTELVETRPAGDGTFQKWAAVVVP
SGEEQRYTCHVQHEGLPKPLTLRWEPSSQPTVPIVGIIAGLVLLGAVITG
AVVAAVMWRRNSSDRKGGSYSQAASSDSAQGSDVSLTACKV (SEQ ID
NO: 1)

In some aspects, a HLA molecule of the disclosure comprises a modified HLA-binding pocket, wherein the HLA molecule is an HLA-A*23 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-A*23 allele, and wherein the modified HLA-binding pocket comprises an amino acid substitution at amino acid residue 81, corresponding to the amino acid sequence set forth in SEQ ID NO: 1.

In some aspects, a HLA molecule of the disclosure comprises a modified HLA-binding pocket, wherein the HLA molecule is an HLA-A*25 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-A*25 allele, and wherein the modified HLA-binding pocket comprises an amino acid substitution at amino acid residue 81, corresponding to the amino acid sequence set forth in SEQ ID NO: 1.

In some aspects, a HLA molecule of the disclosure comprises a modified HLA-binding pocket, wherein the HLA molecule is an HLA-A*31 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-A*31 allele, and wherein the modified HLA-binding pocket comprises an amino acid substitution at amino acid residue 81, corresponding to the amino acid sequence set forth in SEQ ID NO: 1.

In some aspects, a HLA molecule of the disclosure comprises a modified HLA-binding pocket, wherein the HLA molecule is an HLA-A*32 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-A*32 allele, and wherein the modified HLA-binding pocket comprises an amino acid substitution at amino acid residue 81, corresponding to the amino acid sequence set forth in SEQ ID NO: 1.

In some aspects, the original amino acid that is replaced is an alanine. In some aspects, the new amino acid that replaces the original amino acid is selected from the group consisting of leucine, valine, isoleucine, methionine, phenylalanine, tyrosine, and tryptophan. In some aspects, the new amino acid that replaces the original amino acid is a leucine. In some aspects, the amino acid substitution is an A81L, corresponding to the amino acid sequence set forth in SEQ ID NO: 1. In certain aspects, the HLA molecule is an HLA-A*24:02 allele comprising a modified HLA-binding pocket, wherein the modified HLA-binding pocket comprises an A81L substitution, corresponding to the amino acid sequence set forth in SEQ ID NO: 1.

### II.A.2. HLA-B Alleles

In some aspects, the HLA molecule is an HLA-B allele. Any HLA-B allele can be used in methods and compositions of the present disclosure. In some aspects, the HLA molecule is an HLA-B allele selected from HLA-B*07, HLA-B*08, HLA-B*13, HLA-B*14, HLA-B*15, HLA-B*18, HLA-B*27, HLA-B*35, HLA-B*37, HLA-B*38, HLA-B*39, HLA-B*40, HLA-B*41, HLA-B*42, HLA-B*44, HLA-B*45, HLA-B*46, HLA-B*47, HLA-B*48, HLA-B*49, HLA-B*50, HLA-B*51, HLA-B*52, HLA-B*53, HLA-B*54, HLA-B*55, HLA-B*56, HLA-B*57, HLA-B*58, HLA-B*59, HLA-B*67, HLA-B*73, HLA-B*78, HLA-B*79, HLA-B*81, HLA-B*82, and HLA-B*83. In some aspects, the HLA molecule is an HLA-B*44. In some aspects, the HLA molecule is an HLA-B*51. In some aspects, the HLA molecule is an HLA-B*58.

In some aspects, a HLA molecule of the disclosure comprises a modified HLA-binding pocket, wherein the HLA molecule is an HLA-B*44 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-B*44 allele, and wherein the modified HLA-binding pocket comprises an amino acid substitution at amino acid residue 81, corresponding to the amino acid sequence set forth in SEQ ID NO: 1.

In some aspects, a HLA molecule of the disclosure comprises a modified HLA-binding pocket, wherein the HLA molecule is an HLA-B*51 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-B*51 allele, and wherein the modified HLA-binding pocket comprises an amino acid substitution at amino acid residue 81, corresponding to the amino acid sequence set forth in SEQ ID NO: 1.

In some aspects, a HLA molecule of the disclosure comprises a modified HLA-binding pocket, wherein the HLA molecule is an HLA-B*58 allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-B*58 allele, and wherein the modified HLA-binding pocket comprises an amino acid substitution at amino acid residue 81, corresponding to the amino acid sequence set forth in SEQ ID NO: 1.

In some aspects, the HLA molecule is an HLA-B allele selected from HLA-B*44:02:01:01, HLA-B*44:02:01:02, HLA-B*44:02:01:03, HLA-B*44:02:01:04, HLA-B*44:02:01:05, HLA-B*44:02:01:06, HLA-B*44:02:01:07, HLA-B*44:02:01:08, HLA-B*44:02:01:09, HLA-B*44:02:01:10, HLA-B*44:02:01:11, HLA-B*44:02:01:12, HLA-B*44:02:01:13, HLA-B*44:02:01:14, HLA-B*44:02:01:15, HLA-B*44:02:01:16, HLA-B*44:02:01:17, HLA-B*44:02:01:18, HLA-B*44:02:01:19, HLA-B*44:02:01:20, HLA-B*44:02:01:21, HLA-B*44:02:01:22, HLA-B*44:02:01:23, HLA-B*44:02:01:24, HLA-B*44:02:01:25, HLA-B*44:02:01:26, HLA-B*44:02:01:27, HLA-B*44:02:01:28, HLA-B*44:02:01:29, HLA-B*44:02:01:30, HLA-B*44:02:01:31, HLA-B*44:02:01:32, HLA-B*44:02:01:33, HLA-B*44:02:01:34, HLA-B*44:02:01:35, HLA-B*44:02:01:36, HLA-B*44:02:01:37, HLA-B*44:02:01:38, HLA-B*44:02:01:39, HLA-B*44:02:01:40, HLA-B*44:02:01:41, HLA-B*44:02:01:42, HLA-B*44:02:01:43, HLA-B*44:02:01:44, HLA-B*44:02:01:45, HLA-B*44:02:01:46, HLA-B*44:02:01:47, HLA-B*44:02:01:48, HLA-B*44:02:01:49, HLA-B*44:02:01:50, HLA-B*44:02:01:51, HLA-B*44:02:01:52, HLA-B*44:02:02, HLA-B*44:02:03, HLA-B*44:02:04, HLA-B*44:02:05, HLA-B*44:02:06, HLA-B*44:02:07, HLA-B*44:02:08, HLA-B*44:02:09, HLA-B*44:02:10, HLA-B*44:02:11, HLA-B*44:02:12, HLA-B*44:02:13, HLA-B*44:02:14, HLA-B*44:02:15, HLA-B*44:02:16, HLA-B*44:02:17, HLA-B*44:02:18, HLA-B*44:02:19, HLA-B*44:02:20, HLA-B*44:02:21, HLA-B*44:02:22, HLA-B*44:02:23, HLA-B*44:02:24, HLA-B*44:02:25, HLA-B*44:02:26, HLA-B*44:02:27, HLA-B*44:02:28, HLA-B*44:02:29, HLA-B*44:02:30, HLA-B*44:02:31, HLA-B*44:02:32, HLA-B*44:02:33, HLA-B*44:02:34, HLA-B*44:02:35, HLA-B*44:02:36, HLA-B*44:02:37, HLA-B*44:02:38, HLA-B*44:02:39, HLA-B*44:02:40, HLA-B*44:02:41, HLA-B*44:02:42, HLA-B*44:02:43, HLA-B*44:02:44, HLA-B*44:02:45, HLA-B*44:02:46, HLA-B*44:02:47, HLA-B*44:02:48, HLA-B*44:02:49:01, HLA-B*44:02:49:02, HLA-B*44:02:50, HLA-B*44:02:51, HLA-B*44:02:52, HLA-B*44:02:53, HLA-B*44:02:54, HLA-B*44:02:55, HLA-B*44:02:56, HLA-B*44:02:57, HLA-B*44:02:58, HLA-B*44:02:59, HLA-B*44:02:60, HLA-B*44:02:61, HLA-B*44:02:62, HLA-B*44:02:63, HLA-B*44:02:64, HLA-B*44:02:65, HLA-B*44:02:66, HLA-B*44:02:67, HLA-B*44:02:68, HLA-B*44:02:69, HLA-B*44:02:70, HLA-B*44:02:71, HLA-B*44:02:72, HLA-B*44:02:73, HLA-B*44:02:74, HLA-B*44:02:75, HLA-B*44:03:01:01, HLA-B*44:03:01:02, HLA-B*44:03:01:03, HLA-B*44:03:01:04, HLA-B*44:03:01:05, HLA-B*44:03:01:06, HLA-B*44:03:01:07, HLA-B*44:03:01:08, HLA-B*44:03:01:09, HLA-B*44:03:01:10, HLA-B*44:03:01:11, HLA-B*44:03:01:12, HLA-B*44:03:01:13, HLA-B*44:03:01:14, HLA-B*44:03:01:15, HLA-B*44:03:01:16, HLA-B*44:03:01:17, HLA-B*44:03:01:18, HLA-B*44:03:01:19, HLA-B*44:03:01:20, HLA-B*44:03:01:21, HLA-B*44:03:01:22, HLA-B*44:03:01:23, HLA-B*44:03:01:24, HLA-B*44:03:01:25, HLA-B*44:03:01:26, HLA-B*44:03:01:27, HLA-B*44:03:01:28, HLA-B*44:03:01:29, HLA-B*44:03:01:30, HLA-B*44:03:01:31, HLA-B*44:03:01:32, HLA-B*44:03:01:33, HLA-B*44:03:01:34, HLA-B*44:03:01:35, HLA-B*44:03:01:36, HLA-B*44:03:01:37, HLA-B*44:03:01:38, HLA-B*44:03:01:39, HLA-B*44:03:01:40, HLA-B*44:03:01:41, HLA-B*44:03:01:42, HLA-B*44:03:01:43, HLA-B*44:03:02:01, HLA-B*44:03:02:02, HLA-B*44:03:02:03, HLA-B*44:03:03, HLA-B*44:03:04, HLA-B*44:03:05, HLA-B*44:03:06, HLA-B*44:03:07, HLA-B*44:03:08, HLA-B*44:03:09, HLA-B*44:03:10, HLA-B*44:03:11, HLA-B*44:03:12, HLA-B*44:03:13, HLA-B*44:03:14, HLA-B*44:03:15, HLA-B*44:03:16, HLA-B*44:03:17, HLA-B*44:03:18, HLA-B*44:03:19, HLA-B*44:03:20, HLA-B*44:03:21, HLA-B*44:03:22, HLA-B*44:03:23, HLA-B*44:03:24, HLA-B*44:03:25, HLA-B*44:03:26, HLA-B*44:03:27, HLA-B*44:03:28, HLA-B*44:03:29, HLA-B*44:03:30, HLA-B*44:03:31, HLA-B*44:03:32, HLA-B*44:03:33, HLA-B*44:03:34, HLA-B*44:03:35, HLA-B*44:03:36, HLA-B*44:03:37, HLA-B*44:03:38, HLA-B*44:03:39, HLA-B*44:03:40, HLA-B*44:03:41, HLA-B*44:03:42, HLA-B*44:03:43, HLA-B*44:03:44, HLA-B*44:03:45, HLA-B*44:03:46, HLA-B*44:03:47, HLA-B*44:03:48, HLA-B*44:03:49, HLA-B*44:03:50, HLA-B*44:03:51, HLA-B*44:03:52, HLA-B*44:03:53, HLA-B*44:03:54, HLA-B*44:03:55, HLA-B*44:03:56, HLA-B*44:03:57, HLA-B*44:03:58, HLA-B*44:04, HLA-B*44:05:01:01, HLA-B*44:05:01:02, HLA-B*44:05:01:03, HLA-B*44:05:01:04, HLA-B*44:05:02, HLA-B*44:05:03, HLA-B*44:05:04, HLA-B*44:05:05, HLA-B*44:06, HLA-B*44:07, HLA-B*44:08, HLA-B*44:09, HLA-B*44:10, HLA-B*44:11, HLA-B*44:12, HLA-B*44:13, HLA-B*44:14, HLA-B*44:15:01:01, HLA-B*44:15:01:02, HLA-B*44:16, HLA-B*44:17, HLA-B*44:18:01:01, HLA-B*44:18:01:02, HLA-B*44:19 19 HLA-B*44:20, HLA-B*44:21, HLA-B*44:22, HLA-B*44:23:01:01, HLA-B*44:23:01:02, HLA-B*44:24, HLA-B*44:25, HLA-B*44:26, HLA-B*44:27:01:01, HLA-B*44:27:01:02, HLA-B*44:27:01:03, HLA-B*44:27:02, HLA-B*44:27:03, HLA-B*44:27:04, HLA-B*44:28:01, HLA-B*44:28:02, HLA-B*44:29, HLA-B*44:30, HLA-B*44:31, HLA-B*44:32, HLA-B*44:33, HLA-B*44:34:01, HLA-B*44:34:02, HLA-B*44:35, HLA-B*44:36, HLA-B*44:37:01, HLA-B*44:37:02, HLA-B*44:38, HLA-B*44:39, HLA-B*44:40, HLA-B*44:41:01, HLA-B*44:41:02, HLA-B*44:42, HLA-B*44:43:01, HLA-B*44:43:02, HLA-B*44:44, HLA-B*44:45, HLA-B*44:46:01, HLA-B*44:46:02, HLA-B*44:47, HLA-B*44:48, HLA-B*44:49, HLA-B*44:50:01, HLA-B*44:50:02, HLA-B*44:50:03, HLA-B*44:51, HLA-B*44:52, HLA-B*44:53:01, HLA-B*44:53:02, HLA-B*44:54, HLA-B*44:55, HLA-B*44:56, HLA-B*44:57, HLA-B*44:58 74 HLA-B*44:59:01, HLA-B*44:59:02, HLA-B*44:60, HLA-B*44:61 45 HLA-B*44:62, HLA-B*44:63, HLA-B*44:64:01, HLA-B*44:64:02, HLA-B*44:65, HLA-B*44:66, HLA-B*44:67, HLA-B*44:68, HLA-B*44:69:01, HLA-B*44:69:02, HLA-B*44:70, HLA-B*44:71, HLA-B*44:72, HLA-B*44:73, HLA-B*44:74, HLA-B*44:75, HLA-B*44:76, HLA-B*44:77, HLA-B*44:78, HLA-B*44:79:01, HLA-B*44:79:02, HLA-B*44:80, HLA-B*44:81, HLA-B*44:82, HLA-B*44:83, HLA-B*44:84:01, HLA-B*44:84:02, HLA-B*44:85:01, HLA-B*44:85:02, HLA-B*44:86, HLA-B*44:87, HLA-B*44:88, HLA-B*44:89, HLA-B*44:90, HLA-B*44:91, HLA-B*44:92, HLA-B*44:93, HLA-B*44:94, HLA-B*44:95, HLA-B*44:96, HLA-B*44:97, HLA-B*44:98, HLA-B*44:99, HLA-B*44:100, HLA-B*44:101, HLA-B*44:102, HLA-B*44:103, HLA-B*44:104, HLA-B*44:105, HLA-B*44:106, HLA-B*44:107, HLA-B*44:108, HLA-B*44:109, HLA-B*44:110, HLA-B*44:111, HLA-B*44:112, HLA-B*44:113, HLA-B*44:114, HLA-B*44:115, HLA-B*44:116, HLA-B*44:117, HLA-B*44:118, HLA-B*44:119, HLA-B*44:120, HLA-B*44:121, HLA-B*44:122, HLA-B*44:123, HLA-B*44:124, HLA-B*44:125, HLA-B*44:126:01, HLA-B*44:126:02, HLA-B*44:127, HLA-B*44:128:01, HLA-B*44:128:02, HLA-B*44:129, HLA-B*44:130, HLA-B*44:131, HLA-B*44:132, HLA-B*44:133, HLA-B*44:134, HLA-B*44:135, HLA-B*44:136, HLA-B*44:137, HLA-B*44:138, HLA-B*44:139, HLA-B*44:140, HLA-B*44:141, HLA-B*44:142, HLA-B*44:143, HLA-B*44:144, HLA-B*44:145, HLA-B*44:146, HLA-B*44:147, HLA-B*44:148, HLA-B*44:149 95 HLA-B*44:150, HLA-B*44:151, HLA-B*44:152, HLA-B*44:153, HLA-B*44:154, HLA-B*44:155, HLA-B*44:156, HLA-B*44:157, HLA-B*44:158, HLA-B*44:159, HLA-B*44:160, HLA-B*44:161, HLA-B*44:162, HLA-B*44:163:01, HLA-B*44:163:02, HLA-B*44:164, HLA-B*44:165, HLA-B*44:166, HLA-B*44:167, HLA-B*44:168, HLA-B*44:169, HLA-B*44:170, HLA-B*44:171 62 HLA-B*44:172, HLA-B*44:173, HLA-B*44:174, HLA-B*44:175, HLA-B*44:176, HLA-B*44:177, HLA-B*44:178, HLA-B*44:179, HLA-B*44:180, HLA-B*44:181, HLA-B*44:182, HLA-B*44:183, HLA-B*44:184, HLA-B*44:185, HLA-B*44:186:01, HLA-B*44:186:02, HLA-B*44:187, HLA-B*44:188, HLA-B*44:189, HLA-B*44:190, HLA-B*44:191, HLA-B*44:192:01, HLA-B*44:192:02, HLA-B*44:192:03, HLA-B*44:192:04, HLA-B*44:193, HLA-B*44:194:01, HLA-B*44:194:02, HLA-B*44:195, HLA-B*44:196, HLA-B*44:197, HLA-B*44:198, HLA-B*44:199, HLA-B*44:200, HLA-B*44:201, HLA-B*44:202, HLA-B*44:203:01, HLA-B*44:203:02, HLA-B*44:204:01, HLA-B*44:204:02, HLA-B*44:205:01, HLA-B*44:205:02, HLA-B*44:206, HLA-B*44:207, HLA-B*44:208, HLA-B*44:209, HLA-B*44:210:01, HLA-B*44:210:02, HLA-B*44:211, HLA-B*44:212, HLA-B*44:213, HLA-B*44:214, HLA-B*44:215, HLA-B*44:216, HLA-B*44:217, HLA-B*44:218, HLA-B*44:219, HLA-B*44:220, HLA-B*44:221, HLA-B*44:222, HLA-B*44:223, HLA-B*44:224, HLA-B*44:225, HLA-B*44:226, HLA-B*44:227, HLA-B*44:228, HLA-B*44:229, HLA-B*44:230, HLA-B*44:231, HLA-B*44:232, HLA-B*44:233, HLA-B*44:234, HLA-B*44:235, HLA-B*44:236, HLA-B*44:237, HLA-B*44:238, HLA-B*44:239, HLA-B*44:240, HLA-B*44:241, HLA-B*44:242, HLA-B*44:243, HLA-B*44:244, HLA-B*44:245, HLA-B*44:247, HLA-B*44:248, HLA-B*44:249, HLA-B*44:250, HLA-B*44:251, HLA-B*44:252, HLA-B*44:253, HLA-B*44:254, HLA-B*44:255, HLA-B*44:256, HLA-B*44:257, HLA-B*44:258, HLA-B*44:259, HLA-B*44:260, HLA-B*44:261, HLA-B*44:262, HLA-B*44:263, HLA-B*44:264, HLA-B*44:265, HLA-B*44:266:01, HLA-B*44:266:02, HLA-B*44:267, HLA-B*44:268, HLA-B*44:269, HLA-B*44:270:01, HLA-B*44:270:02, HLA-B*44:271, HLA-B*44:272, HLA-B*44:273, HLA-B*44:274, HLA-B*44:275, HLA-B*44:276, HLA-B*44:277, HLA-B*44:278, HLA-B*44:279, HLA-B*44:280, HLA-B*44:281:01, HLA-B*44:281:02, HLA-B*44:282, HLA-B*44:283, HLA-B*44:284, HLA-B*44:285, HLA-B*44:286, HLA-B*44:287, HLA-B*44:288, HLA-B*44:289, HLA-B*44:290, HLA-B*44:291, HLA-B*44:292, HLA-B*44:293, HLA-B*44:294, HLA-B*44:295, HLA-B*44:296, HLA-B*44:297, HLA-B*44:298, HLA-B*44:299, HLA-B*44:300, HLA-B*44:301, HLA-B*44:302, HLA-B*44:303, HLA-B*44:304, HLA-B*44:305, HLA-B*44:306 7 HLA-B*44:307, HLA-B*44:308, HLA-B*44:309, HLA-B*44:310, HLA-B*44:311, HLA-B*44:312, HLA-B*44:313, HLA-B*44:314, HLA-B*44:315, HLA-B*44:316, HLA-B*44:317, HLA-B*44:318, HLA-B*44:319, HLA-B*44:320, HLA-B*44:321, HLA-B*44:322, HLA-B*44:323, HLA-B*44:324, HLA-B*44:325, HLA-B*44:326, HLA-B*44:327, HLA-B*44:328 79 HLA-B*44:329, HLA-B*44:330, HLA-B*44:331, HLA-B*44:332, HLA-B*44:333, HLA-B*44:334, HLA-B*44:335, HLA-B*44:336, HLA-B*44:337, HLA-B*44:338, HLA-B*44:339, HLA-B*44:340, HLA-B*44:341, HLA-B*44:342, HLA-B*44:343, HLA-B*44:344, HLA-B*44:345, HLA-B*44:346, HLA-B*44:437, HLA-B*44:438, HLA-B*44:439, HLA-B*44:440, HLA-B*44:441, HLA-B*44:442, HLA-B*44:443, HLA-B*44:444, HLA-B*44:445, HLA-B*44:446, HLA-B*44:447, HLA-B*44:448, HLA-B*44:449 76 HLA-B*44:450, HLA-B*44:451, HLA-B*44:452, HLA-B*44:453, HLA-B*44:454, HLA-B*44:455, HLA-B*44:456, HLA-B*44:457, HLA-B*44:458, HLA-B*44:459, HLA-B*44:460, HLA-B*44:461, HLA-B*44:462, HLA-B*44:463, HLA-B*44:464:01:01, HLA-B*44:464:01:02, HLA-B*44:465, HLA-B*44:467, HLA-B*44:468, HLA-B*44:469, HLA-B*44:470, HLA-B*44:471, HLA-B*44:472, HLA-B*44:473, HLA-B*44:474, HLA-B*44:475, HLA-B*44:476, HLA-B*44:477, HLA-B*44:478, HLA-B*44:479, HLA-B*44:480, HLA-B*44:481, HLA-B*44:482, HLA-B*44:483, HLA-B*44:484, HLA-B*44:485, HLA-B*44:486, HLA-B*44:487, HLA-B*44:488, HLA-B*44:489, HLA-B*44:490, HLA-B*44:491, HLA-B*44:492, HLA-B*44:493, HLA-B*44:494, HLA-B*44:495, HLA-B*44:496, HLA-B*44:497, HLA-B*44:498, HLA-B*44:499, HLA-B*44:500, HLA-B*44:501, HLA-B*44:502, HLA-B*44:503, HLA-B*44:504, HLA-B*44:505, HLA-B*44:506, HLA-B*44:507, HLA-B*44:508, HLA-B*44:509, HLA-B*44:510, HLA-B*44:511, HLA-B*44:512, HLA-B*44:513, HLA-B*44:514, HLA-B*44:515, HLA-B*44:516, HLA-B*44:517, HLA-B*44:518, HLA-B*44:519, HLA-B*44:520, HLA-B*44:521, HLA-B*44:522, HLA-B*44:523, and HLA-B*44:524.

In some aspects, the HLA molecule is an HLA-B allele selected from HLA-B*51:01:01:01, HLA-B*51:01:01:02, HLA-B*51:01:01:03, HLA-B*51:01:01:04, HLA-B*51:01:01:05, HLA-B*51:01:01:06, HLA-B*51:01:01:07, HLA-B*51:01:01:08, HLA-B*51:01:01:09, HLA-B*51:01:01:10, HLA-B*51:01:01:11, HLA-B*51:01:01:12, HLA-B*51:01:01:13, HLA-B*51:01:01:14, HLA-B*51:01:01:15, HLA-B*51:01:01:16, HLA-B*51:01:01:17, HLA-B*51:01:01:18, HLA-B*51:01:01:19, HLA-B*51:01:01:20, HLA-B*51:01:01:21, HLA-B*51:01:01:22, HLA-B*51:01:01:23, HLA-B*51:01:01:24, HLA-B*51:01:01:25, HLA-B*51:01:01:26, HLA-B*51:01:01:27, HLA-B*51:01:01:28, HLA-B*51:01:01:29, HLA-B*51:01:01:30, HLA-B*51:01:01:31, HLA-B*51:01:01:32, HLA-B*51:01:01:33, HLA-B*51:01:01:34, HLA-B*51:01:01:35, HLA-B*51:01:01:36, HLA-B*51:01:01:37, HLA-B*51:01:01:38, HLA-B*51:01:01:39, HLA-B*51:01:01:40, HLA-B*51:01:01:41, HLA-B*51:01:01:42, HLA-B*51:01:01:43, HLA-B*51:01:01:44, HLA-B*51:01:01:45, HLA-B*51:01:01:46, HLA-B*51:01:01:47, HLA-B*51:01:01:48, HLA-B*51:01:01:49, HLA-B*51:01:01:50, HLA-B*51:01:01:51, HLA-B*51:01:01:52, HLA-B*51:01:01:53, HLA-B*51:01:01:54, HLA-B*51:01:01:55, HLA-B*51:01:01:56, HLA-B*51:01:01:57, HLA-B*51:01:01:58, HLA-B*51:01:01:59, HLA-B*51:01:01:60, HLA-B*51:01:01:61, HLA-B*51:01:01:62, HLA-B*51:01:01:63, HLA-B*51:01:01:64, HLA-B*51:01:01:65, HLA-B*51:01:01:66, HLA-B*51:01:01:67, HLA-B*51:01:01:68, HLA-B*51:01:01:69, HLA-B*51:01:01:70, HLA-B*51:01:01:71, HLA-B*51:01:01:72, HLA-B*51:01:01:73, HLA-B*51:01:01:74, HLA-B*51:01:01:75, HLA-B*51:01:01:76, HLA-B*51:01:01:77, HLA-B*51:01:01:78, HLA-B*51:01:02:01, HLA-B*51:01:02:02, HLA-B*51:01:03, HLA-B*51:01:04, HLA-B*51:01:05, HLA-B*51:01:06, HLA-B*51:01:07, HLA-B*51:01:08, HLA-B*51:01:09, HLA-B*51:01:10, HLA-B*51:01:11, HLA-B*51:01:12, HLA-B*51:01:13, HLA-B*51:01:14, HLA-B*51:01:15, HLA-B*51:01:16, HLA-B*51:01:17, HLA-B*51:01:18, HLA-B*51:01:19, HLA-B*51:01:20, HLA-B*51:01:21, HLA-B*51:01:22, HLA-B*51:01:23, HLA-B*51:01:24, HLA-B*51:01:25, HLA-B*51:01:26, HLA-B*51:01:27, HLA-B*51:01:28, HLA-B*51:01:29, HLA-B*51:01:30, HLA-B*51:01:31, HLA-B*51:01:32, HLA-B*51:01:33, HLA-B*51:01:34, HLA-B*51:01:35, HLA-B*51:01:36, HLA-B*51:01:37, HLA-B*51:01:38, HLA-B*51:01:39, HLA-B*51:01:40, HLA-B*51:01:41, HLA-B*51:01:42, HLA-B*51:01:43, HLA-B*51:01:44, HLA-B*51:01:45, HLA-B*51:01:46, HLA-B*51:01:47, HLA-B*51:01:48, HLA-B*51:01:49, HLA-B*51:01:50, HLA-B*51:01:51, HLA-B*51:01:52, HLA-B*51:01:53, HLA-B*51:01:54, HLA-B*51:01:55, HLA-B*51:01:56, HLA-B*51:01:57, HLA-B*51:01:58, HLA-B*51:01:59, HLA-B*51:01:60, HLA-B*51:01:61, HLA-B*51:01:62, HLA-B*51:01:63, HLA-B*51:01:64, HLA-B*51:01:65, HLA-B*51:01:66, HLA-B*51:01:67, HLA-B*51:01:68, HLA-B*51:01:69, HLA-B*51:01:70, HLA-B*51:01:71, HLA-B*51:01:72, HLA-B*51:01:73, HLA-B*51:01:74, HLA-B*51:01:75, HLA-B*51:01:76, HLA-B*51:01:77, HLA-B*51:01:78, HLA-B*51:01:79, HLA-B*51:01:80, HLA-B*51:01:81, HLA-B*51:01:82, HLA-B*51:01:83, HLA-B*51:01:84, HLA-B*51:01:85, HLA-B*51:01:86, HLA-B*51:01:87, HLA-B*51:01:88, HLA-B*51:01:89, HLA-B*51:01:90, HLA-B*51:01:91, HLA-B*51:01:92, HLA-B*51:02:01:01, HLA-B*51:02:01:02, HLA-B*51:02:01:03, HLA-B*51:02:02, HLA-B*51:02:03, HLA-B*51:02:04, HLA-B*51:02:05, HLA-B*51:02:06, HLA-B*51:02:07, HLA-B*51:03, HLA-B*51:04:01, HLA-B*51:04:02, HLA-B*51:05, HLA-B*51:06:01:01, HLA-B*51:06:01:02, HLA-B*51:06:01:03, HLA-B*51:06:02, HLA-B*51:06:03, HLA-B*51:06:04, HLA-B*51:07:01, HLA-B*51:07:02, HLA-B*51:08:01:01, HLA-B*51:08:01:02, HLA-B*51:08:01:03, HLA-B*51:08:01:04, HLA-B*51:08:02, HLA-B*51:08:03, HLA-B*51:08:04, HLA-B*51:08:05, HLA-B*51:09:01, HLA-B*51:09:02, HLA-B*51:09:03, HLA-B*51:10, HLA-B*51:11, HLA-B*51:12, HLA-B*51:13:01, HLA-B*51:13:02, HLA-B*51:14, HLA-B*51:15, HLA-B*51:16, HLA-B*51:17, HLA-B*51:18, HLA-B*51:19, HLA-B*51:20, HLA-B*51:21, HLA-B*51:22, HLA-B*51:23, HLA-B*51:24:01, HLA-B*51:24:02, HLA-B*51:24:03, HLA-B*51:24:04, HLA-B*51:24:05, HLA-B*51:26, HLA-B*51:27, HLA-B*51:28, HLA-B*51:29, HLA-B*51:30, HLA-B*51:31, HLA-B*51:32, HLA-B*51:33, HLA-B*51:34, HLA-B*51:35, HLA-B*51:36, HLA-B*51:37, HLA-B*51:38, HLA-B*51:39, HLA-B*51:40, HLA-B*51:41 74 HLA-B*51:42, HLA-B*51:43, HLA-B*51:44 64 HLA-B*51:45, HLA-B*51:46, HLA-B*51:48, HLA-B*51:49, HLA-B*51:50, HLA-B*51:51, HLA-B*51:52, HLA-B*51:53, HLA-B*51:54, HLA-B*51:55, HLA-B*51:56:01, HLA-B*51:56:02, HLA-B*51:56:03, HLA-B*51:57, HLA-B*51:58, HLA-B*51:59, HLA-B*51:60, HLA-B*51:61:01, HLA-B*51:61:02, HLA-B*51:62, HLA-B*51:63:01, HLA-B*51:63:02, HLA-B*51:64, HLA-B*51:65, HLA-B*51:66, HLA-B*51:67, HLA-B*51:68, HLA-B*51:69, HLA-B*51:70, HLA-B*51:71, HLA-B*51:72, HLA-B*51:73, HLA-B*51:74, HLA-B*51:75, HLA-B*51:76, HLA-B*51:77, HLA-B*51:78:01, HLA-B*51:78:02, HLA-B*51:79, HLA-B*51:80, HLA-B*51:81, HLA-B*51:82, HLA-B*51:83, HLA-B*51:84, HLA-B*51:85, HLA-B*51:86, HLA-B*51:87, HLA-B*51:88, HLA-B*51:89, HLA-B*51:90, HLA-B*51:91, HLA-B*51:92:01, HLA-B*51:92:02, HLA-B*51:93, HLA-B*51:94, HLA-B*51:95, HLA-B*51:96, HLA-B*51:97, HLA-B*51:98 18 HLA-B*51:99, HLA-B*51:100, HLA-B*51:101, HLA-B*51:102, HLA-B*51:103, HLA-B*51:104:01, HLA-B*51:104:02, HLA-B*51:105, HLA-B*51:106:01, HLA-B*51:106:02, HLA-B*51:107, HLA-B*51:108, HLA-B*51:109, HLA-B*51:110 84 HLA-B*51:111, HLA-B*51:112, HLA-B*51:113, HLA-B*51:114, HLA-B*51:115, HLA-B*51:116, HLA-B*51:117, HLA-B*51:118, HLA-B*51:119, HLA-B*51:120, HLA-B*51:121, HLA-B*51:122, HLA-B*51:123, HLA-B*51:124, HLA-B*51:125, HLA-B*51:126, HLA-B*51:127, HLA-B*51:128, HLA-B*51:129, HLA-B*51:130, HLA-B*51:131, HLA-B*51:132, HLA-B*51:133, HLA-B*51:134, HLA-B*51:135, HLA-B*51:136, HLA-B*51:137, HLA-B*51:138, HLA-B*51:139, HLA-B*51:140, HLA-B*51:141, HLA-B*51:142, HLA-B*51:143, HLA-B*51:144, HLA-B*51:145, HLA-B*51:146, HLA-B*51:147, HLA-B*51:148, HLA-B*51:149, HLA-B*51:150, HLA-B*51:151, HLA-B*51:152, HLA-B*51:153, HLA-B*51:154, HLA-B*51:155, HLA-B*51:156, HLA-B*51:157, HLA-B*51:158:01, HLA-B*51:158:02, HLA-B*51:159, HLA-B*51:160, HLA-B*51:161, HLA-B*51:162:01, HLA-B*51:162:02, HLA-B*51:163, HLA-B*51:164, HLA-B*51:165, HLA-B*51:166, HLA-B*51:167, HLA-B*51:168, HLA-B*51:169, HLA-B*51:170, HLA-B*51:171, HLA-B*51:172, HLA-B*51:173, HLA-B*51:174:01, HLA-B*51:174:02, HLA-B*51:175, HLA-B*51:176, HLA-B*51:177, HLA-B*51:178 57 HLA-B*51:179, HLA-B*51:180, HLA-B*51:181, HLA-B*51:182, HLA-B*51:183, HLA-B*51:184 31 HLA-B*51:185, HLA-B*51:186, HLA-B*51:187, HLA-B*51:188, HLA-B*51:189, HLA-B*51:190, HLA-B*51:191, HLA-B*51:192, HLA-B*51:193, HLA-B*51:194, HLA-B*51:195, HLA-B*51:196, HLA-B*51:197, HLA-B*51:198, HLA-B*51:199, HLA-B*51:200, HLA-B*51:201, HLA-B*51:202, HLA-B*51:203, HLA-B*51:204:01, HLA-B*51:204:02, HLA-B*51:205, HLA-B*51:206, HLA-B*51:207, HLA-B*51:208, HLA-B*51:209, HLA-B*51:210, HLA-B*51:211, HLA-B*51:212, HLA-B*51:213, HLA-B*51:214, HLA-B*51:215, HLA-B*51:216, HLA-B*51:217, HLA-B*51:218, HLA-B*51:219, HLA-B*51:220, HLA-B*51:221, HLA-B*51:222, HLA-B*51:223, HLA-B*51:224, HLA-B*51:225, HLA-B*51:226, HLA-B*51:227, HLA-B*51:228, HLA-B*51:229, HLA-B*51:230:01:01, HLA-B*51:230:01:02, HLA-B*51:230:01:03, HLA-B*51:231, HLA-B*51:232:01, HLA-B*51:232:02, HLA-B*51:233, HLA-B*51:234, HLA-B*51:235, HLA-B*51:236, HLA-B*51:237:01, HLA-B*51:237:02, HLA-B*51:238, HLA-B*51:239, HLA-B*51:240, HLA-B*51:241, HLA-B*51:242, HLA-B*51:243, HLA-B*51:244, HLA-B*51:245 64 HLA-B*51:246, HLA-B*51:247, HLA-B*51:248, HLA-B*51:249, HLA-B*51:250, HLA-B*51:251, HLA-B*51:252, HLA-B*51:253, HLA-B*51:254, HLA-B*51:255, HLA-B*51:256, HLA-B*51:257, HLA-B*51:258, HLA-B*51:259, HLA-B*51:260, HLA-B*51:261, HLA-B*51:262, HLA-B*51:263, HLA-B*51:264, HLA-B*51:265, HLA-B*51:266, HLA-B*51:267, HLA-B*51:268, HLA-B*51:269, HLA-B*51:270, HLA-B*51:271, HLA-B*51:272, HLA-B*51:273, HLA-B*51:274, HLA-B*51:275, HLA-B*51:276, HLA-B*51:277, HLA-B*51:278, HLA-B*51:279, HLA-B*51:280, HLA-B*51:281, HLA-B*51:282, HLA-B*51:283, HLA-B*51:284, HLA-B*51:285, HLA-B*51:286, HLA-B*51:287, HLA-B*51:288, HLA-B*51:289, HLA-B*51:290, HLA-B*51:291, HLA-B*51:292, HLA-B*51:293, HLA-B*51:294, HLA-B*51:295, HLA-B*51:296, HLA-B*51:297, HLA-B*51:298, HLA-B*51:299, HLA-B*51:300, HLA-B*51:301, HLA-B*51:302, HLA-B*51:303, HLA-B*51:304, HLA-B*51:305, HLA-B*51:306, HLA-B*51:307, HLA-B*51:308, HLA-B*51:309, HLA-B*51:310, HLA-B*51:311, HLA-B*51:312, HLA-B*51:313, HLA-B*51:314, HLA-B*51:315, HLA-B*51:316, HLA-B*51:317, HLA-B*51:318, HLA-B*51:319, HLA-B*51:320, HLA-B*51:321, HLA-B*51:322, HLA-B*51:323, HLA-B*51:324, HLA-B*51:325, HLA-B*51:326, HLA-B*51:327, HLA-B*51:328, HLA-B*51:329, HLA-B*51:330, HLA-B*51:331, HLA-B*51:332, HLA-B*51:333, HLA-B*51:334, HLA-B*51:335, HLA-B*51:336, HLA-B*51:337, HLA-B*51:338, HLA-B*51:339, HLA-B*51:340, HLA-B*51:341, HLA-B*51:342, HLA-B*51:343, HLA-B*51:344 31, and HLA-B*51:345.

In some aspects, the HLA molecule is an HLA-B allele selected from HLA-B*58:01:01:01, HLA-B*58:01:01:02, HLA-B*58:01:01:03, HLA-B*58:01:01:04, HLA-B*58:01:01:05, HLA-B*58:01:01:06, HLA-B*58:01:01:07, HLA-B*58:01:01:08, HLA-B*58:01:01:09, HLA-B*58:01:01:10, HLA-B*58:01:01:11, HLA-B*58:01:01:12, HLA-B*58:01:01:13, HLA-B*58:01:02, HLA-B*58:01:03, HLA-B*58:01:04, HLA-B*58:01:05, HLA-B*58:01:06, HLA-B*58:01:07, HLA-B*58:01:08, HLA-B*58:01:09, HLA-B*58:01:10, HLA-B*58:01:11, HLA-B*58:01:12, HLA-B*58:01:13, HLA-B*58:01:14, HLA-B*58:01:15, HLA-B*58:01:16, HLA-B*58:01:17, HLA-B*58:01:18, HLA-B*58:01:19, HLA-B*58:01:20, HLA-B*58:01:21, HLA-B*58:01:22, HLA-B*58:01:23, HLA-B*58:01:24, HLA-B*58:01:25, HLA-B*58:01:26, HLA-B*58:01:27, HLA-B*58:01:28, HLA-B*58:01:29, HLA-B*58:01:30, HLA-B*58:01:31, HLA-B*58:01:32, HLA-B*58:01:33, HLA-B*58:01:34, HLA-B*58:01:35, HLA-B*58:01:36, HLA-B*58:01:37, HLA-B*58:01:38, HLA-B*58:01:39, HLA-B*58:02:01:01, HLA-B*58:02:01:02, HLA-B*58:02:01:03, HLA-B*58:02:02, HLA-B*58:04, HLA-B*58:05, HLA-B*58:06, HLA-B*58:07, HLA-B*58:08:01, HLA-B*58:08:02, HLA-B*58:09, HLA-B*58:10, HLA-B*58:11, HLA-B*58:12, HLA-B*58:13, HLA-B*58:14, HLA-B*58:15, HLA-B*58:16:01, HLA-B*58:16:02, HLA-B*58:17, HLA-B*58:18, HLA-B*58:19, HLA-B*58:20, HLA-B*58:21, HLA-B*58:22, HLA-B*58:23, HLA-B*58:24, HLA-B*58:25, HLA-B*58:26, HLA-B*58:27, HLA-B*58:28:01, HLA-B*58:28:02, HLA-B*58:29, HLA-B*58:31, HLA-B*58:32, HLA-B*58:33, HLA-B*58:34, HLA-B*58:35, HLA-B*58:36, HLA-B*58:37, HLA-B*58:38, HLA-B*58:39 45 HLA-B*58:40, HLA-B*58:41, HLA-B*58:42, HLA-B*58:43, HLA-B*58:44, HLA-B*58:45:01, HLA-B*58:45:02, HLA-B*58:46, HLA-B*58:47, HLA-B*58:48, HLA-B*58:49, HLA-B*58:50, HLA-B*58:51, HLA-B*58:52, HLA-B*58:53, HLA-B*58:54, HLA-B*58:55, HLA-B*58:56, HLA-B*58:57, HLA-B*58:58, HLA-B*58:59:01, HLA-B*58:59:02, HLA-B*58:60, HLA-B*58:61, HLA-B*58:62, HLA-B*58:63, HLA-B*58:64, HLA-B*58:65, HLA-B*58:66, HLA-B*58:67, HLA-B*58:68, HLA-B*58:69, HLA-B*58:70, HLA-B*58:71, HLA-B*58:72, HLA-B*58:73, HLA-B*58:74, HLA-B*58:75, HLA-B*58:76, HLA-B*58:77, HLA-B*58:78, HLA-B*58:79, HLA-B*58:80, HLA-B*58:81, HLA-B*58:82, HLA-B*58:83, HLA-B*58:84, HLA-B*58:85, HLA-B*58:86, HLA-B*58:87, HLA-B*58:88, HLA-B*58:89, HLA-B*58:90, HLA-B*58:91, HLA-B*58:92, HLA-B*58:93, HLA-B*58:94, HLA-B*58:95, HLA-B*58:96, HLA-B*58:97, HLA-B*58:98, HLA-B*58:99, HLA-B*58:100, HLA-B*58:101, HLA-B*58:102, HLA-B*58:103, HLA-B*58:104, HLA-B*58:105, HLA-B*58:106, HLA-B*58:107, HLA-B*58:108, HLA-B*58:109, HLA-B*58:110, HLA-B*58:111, HLA-B*58:112, HLA-B*58:113, HLA-B*58:114, HLA-B*58:115, HLA-B*58:116, HLA-B*58:117, HLA-B*58:118, HLA-B*58:119, HLA-B*58:120, HLA-B*58:121, HLA-B*58:122, HLA-B*58:123, HLA-B*58:124, HLA-B*58:125, HLA-B*58:126, HLA-B*58:127, HLA-B*58:128, HLA-B*58:129, HLA-B*58:130, HLA-B*58:131, HLA-B*58:132, HLA-B*58:133,

In some aspects, a HLA molecule of the disclosure comprises a modified HLA-binding pocket, wherein the HLA molecule is an HLA-B allele. In some aspects, the modified HLA-binding pocket is the F pocket of the HLA molecule. In some aspects, a HLA molecule of the disclosure comprises a modified HLA-binding pocket, wherein the HLA molecule is an HLA-B allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-B allele, and wherein the modified HLA-binding pocket comprises an amino acid substitution at amino acid residue 81, corresponding to the amino acid sequence set forth in SEQ ID NO: 1.

In some aspects, the original amino acid that is replaced is an alanine. In some aspects, the new amino acid that replaces the original amino acid is selected from the group consisting of leucine, valine, isoleucine, methionine, phenylalanine, tyrosine, and tryptophan. In some aspects, the new amino acid that replaces the original amino acid is a leucine. In some aspects, the amino acid substitution is an A81L, corresponding to the amino acid sequence set forth in SEQ ID NO: 1.

### II.A.3. HLA-C Alleles

In some aspects, the HLA class I molecule is an HLA-C allele. Any HLA-C allele can be used in methods and compositions of the present disclosure. In some aspects, the HLA-C allele selected from an HLA-C*05:01 allele, an HLA-C*05:03 allele, an HLA-C*05:04 allele, an HLA-C*05:05 allele, and an HLA-C*05:06 allele. In certain aspects, the HLA-C allele is an HLA-C*05:01 allele. In certain aspects, the HLA-C allele is an HLA-C*05:03 allele. In certain aspects, the HLA-C allele is an HLA-C*05:04 allele. In certain aspects, the HLA-C allele is an HLA-C*05:05 allele. In certain aspects, the HLA-C allele is an HLA-C*05:06 allele.

In some aspects, a HLA molecule of the disclosure comprises a modified HLA-binding pocket, wherein the HLA molecule is an HLA-C allele. In some aspects, the modified HLA-binding pocket is the F pocket of the HLA molecule. In some aspects, a HLA molecule of the disclosure comprises a modified HLA-binding pocket, wherein the HLA molecule is an HLA-C allele, and wherein the modified HLA-binding pocket comprises an amino acid substitution at a position corresponding to amino acid residue 79, amino acid residue 80, amino acid residue 81, amino acid residue 82, or amino acid residue 83 of SEQ ID NO: 1. In some aspects, a HLA molecule of the disclosure comprises a modified HLA-binding pocket, wherein the HLA molecule is an HLA-C allele, and wherein the modified HLA-binding pocket comprises an amino acid substitution at amino acid residue 81, corresponding to the amino acid sequence set forth in SEQ ID NO: 1.

In some aspects, the original amino acid that is replaced is an alanine. In some aspects, the new amino acid that replaces the original amino acid is selected from the group consisting of leucine, valine, isoleucine, methionine, phenylalanine, tyrosine, and tryptophan. In some aspects, the new amino acid that replaces the original amino acid is a leucine. In some aspects, the amino acid substitution is an A81L, corresponding to the amino acid sequence set forth in SEQ ID NO: 1.

### II.A.3. HLA Class I Binding Pockets

Various aspects of the present disclosure are directed to HLA molecules comprising a modified HLA-binding pocket. An HLA class I molecule comprises 6 binding pockets: A, B, C, D, E, and F. Data presented herein illustrate that modification of the amino acid sequence of one or more residues within one or more the 6 binding pockets can enhance the binding affinity of the HLA molecule to a target antigen. In doing so, the present disclosure provides novel methods of increasing antigen-binding affinity of HLA molecules that can be applied to a plethora of different HLA alleles.

In some aspects, the HLA molecule comprises a modified F pocket. In some aspects, the HLA molecule comprises a modified A pocket. In some aspects, the HLA molecule comprises a modified B pocket. In some aspects, the HLA molecule comprises a modified C pocket. In some aspects, the HLA molecule comprises a modified D pocket. In some aspects, the HLA molecule comprises a modified E pocket.

In some aspects, more than one HLA-binding pocket of the HLA molecule is modified. In some aspects, the HLA molecule comprises (i) a modified F pocket and (ii) a modified A pocket. In some aspects, the HLA molecule comprises (i) a modified F pocket and (ii) a modified B pocket. In some aspects, the HLA molecule comprises (i) a modified F pocket and (ii) a modified C pocket. In some aspects, the HLA molecule comprises (i) a modified F pocket and (ii) a modified D pocket. In some aspects, the HLA molecule comprises (i) a modified F pocket and (ii) a modified E pocket.

In some aspects, the HLA molecule comprises (i) a modified F pocket, (ii) a modified B pocket, and (iii) a modified A pocket. In some aspects, the HLA molecule comprises (i) a modified F pocket, (ii) a modified B pocket, and (iii) a modified C pocket. In some aspects, the HLA molecule comprises (i) a modified F pocket, (ii) a modified B pocket, and (iii) a modified D pocket. In some aspects, the HLA molecule comprises (i) a modified F pocket, (ii) a modified B pocket, and (iii) a modified E pocket.

In some aspects, the HLA molecule comprises (i) a modified F pocket, (ii) a modified A pocket, and (iii) a modified C pocket. In some aspects, the HLA molecule comprises (i) a modified F pocket, (ii) a modified A pocket, and (iii) a modified D pocket. In some aspects, the HLA molecule comprises (i) a modified F pocket, (ii) a modified A pocket, and (iii) a modified E pocket.

In some aspects, the HLA molecule comprises (i) a modified F pocket, (ii) a modified C pocket, and (iii) a modified D pocket. In some aspects, the HLA molecule comprises (i) a modified F pocket, (ii) a modified C pocket, and (iii) a modified E pocket.

In some aspects, the HLA molecule comprises (i) a modified F pocket, (ii) a modified D pocket, and (iii) a modified E pocket.

### II.B. Methods of Treating Cancer

Certain aspects of the present disclosure are directed to methods of treating a cancer in a subject in need thereof, comprising administering to the subject a nucleic acid molecule disclosed herein, a recombinant TCR disclosed herein, a bispecific TCR disclosed herein, an epitope disclosed herein, or an HLA class I molecule disclosed herein, or a vector or cell comprising any of the above.

In some aspects, the cancer is selected from melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. In some aspects, the cancer melanoma.

In some aspects, the cancer is relapsed. In some aspects, the cancer is refractory. In some aspects, the cancer is advanced. In some aspects, the cancer is metastatic.

In some aspects, the methods disclosed herein treat a cancer in a subject. In some aspects, the methods disclosed herein reduce the severity of one or more symptom of the cancer. In some aspects, the methods disclosed herein reduce the size or number of a tumor derived from the cancer. In some aspects, the methods disclosed herein increase the overall survival of the subject, relative to a subject not provided the methods disclosed herein. In some aspects, the methods disclosed herein increase the progressive-free survival of the subject, relative to a subject not provided the methods disclosed herein. In some aspects, the methods disclosed herein lead to a partial response in the subject. In some aspects, the methods disclosed herein lead to a complete response in the subject.

In some aspects, the methods disclosed herein comprise treating a cancer in a subject in need thereof, comprising administering to the subject a cell described herein, wherein the cell comprises a nucleic acid molecule disclosed herein, a vector disclosed herein, a recombinant TCR disclosed herein, and/or a bispecific antibody disclosed herein. In some aspects, the cell is a T cell. In some aspects, the cell is a cell that is modified to express CD3.

In some aspects, the cell, e.g., a T cell, is obtained from the subject. In some aspects, the cell, e.g., a T cell, is obtained from a donor other than the subject.

In some aspects, the subject is preconditioned prior to administering the cells. The preconditioning can comprise any substance that promotes T cell function and/or survival. In some aspects, the preconditioning comprises administering to the subject a chemotherapy, a cytokine, a protein, a small molecule, or any combination thereof. In some aspects, the preconditioning comprises administering an interleukin. In some aspects, the preconditioning comprises administering IL-2, IL-4, IL-7, IL-9, IL-15, IL-21, or any combination thereof. In some aspects, the preconditioning comprises administering cyclophosphamide, fludarabine, or both. In some aspects, the preconditioning comprises administering vitamin C, an AKT inhibitor, ATRA (vesanoid, tretinoin), rapamycin, or any combination thereof.

In some aspects, a composition disclosed herein (e.g., an antigen-HLA complex, a cell expressing a modified HLA, or a vaccine disclosed herein) is administered to a subject in combination with an immunotherapy. Without being bound by any particular mechanism, modification of the HLA binding pocket, according to the present disclosure, increases the affinity of the HLA for an antigen, thereby increasing the surface display of the antigen on a cell expressing the modified HLA. This increased surface display enhances an immune response to the antigen. As such, the modified HLA molecules disclosed herein can act to enhance an immune response to an immunotherapy.

Any immunotherapy can benefit from coadministration with the modified HLA molecules disclosed herein. As used herein, “coadministration” refers to at least two therapies being administered within a set period of time. In some aspects, the at least two therapies are administered concurrently (e.g., at the same time). In some aspects, the at least two therapies are administered sequentially (e.g., one after the other). In some aspects, the at least two therapies are administered on the same day. In some aspects, the at least two therapies are administered on consecutive days. In some aspects, the at least two therapies are administered during the same dosing cycle (e.g., according to the prescribed dosing regimen of the immunotherapy).

In some aspects, the immunotherapy comprises administering a plurality of immune cells to the subject (i.e., an immune cell therapy or a cell-based therapy). Immune cell therapies have emerged as a promising means of treating various conditions, including cancer. In some aspects, the immune cell therapy comprises administering a plurality of T cells, NK cells, tumor-infiltrating lymphocytes (TILs), or any combination thereof. In some aspects, the immune cells are modified. In some aspects, the immune cells are modified to express a chimeric antigen receptor (CAR), a heterologous T cell receptor (TCR), an engineered TCR, or any combination thereof. In some aspects, the immune cell therapy comprises administering engineered T cell, wherein the engineered T cell comprises a nucleic acid molecule encoding a CAR, a heterologous TCR, an engineered TCR, or any combination thereof. In some aspects, the immune cell therapy comprises administering engineered NK cell, wherein the engineered NK cell comprises a nucleic acid molecule encoding a CAR, a heterologous TCR, an engineered TCR, or any combination thereof.

In some aspects, the immunotherapy comprises an antagonist (inhibitor or blocking agent) of a protein that inhibits T cell activation (e.g., immune checkpoint inhibitors), such as CTLA-4, PD-1, PD-L1, PD-L2, GITR, LAG-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, mesothelin, CD27, CD96, TIM-1, TIM-3, and TIM-4. In some aspects, the immunotherapy comprises a PD-1 inhibitor, a PD-L1 inhibitor, a CTLA-4 inhibitor, a LAG3 inhibitor, a TIM3 inhibitor, or any combination thereof.

In some aspects, the immunotherapy comprises a cancer vaccine.

In some aspects, the immunotherapy comprises an antibody or an antigen-binding portion thereof that specifically binds PD-1 or PD-L1. In some aspects, the immunotherapy comprises an anti-PD-1 antibody selected from nivolumab (OPDIVO®) and pembrolizumab (KEYTRUDA®). In some aspects, the immunotherapy is selected from YERVOY® (ipilimumab) or Tremelimumab (to CTLA-4), galiximab (to B7.1), BMS-936558 (to PD-1), MK-3475 (to PD-1), atezolizumab (TECENTRIQ®), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3); anti-GITR antibodies MK4166, TRX518, Medi1873, INBRX-110, LK2-145, GWN-323, GITRL-Fc, and any combination thereof.

In some aspects, the immunotherapy comprises an agent that targets (or binds specifically to) a member of the B7 family of membrane-bound ligands that includes B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor or ligand binding specifically to a B7 family member. In some aspects, the immunotherapy comprises an agonist of a protein that stimulates T cell activation, such as B7-1, B7-2, CD28, 4-1BB (CD137), 4-1BBL, GITR, ICOS, ICOS-L, OX40, OX40L, CD70, CD27, CD40, DR3 and CD28H. In some aspects, the immunotherapy comprises an antagonist of an inhibitory receptor on NK cells or an agonist of an activating receptor on NK cells, e.g., an antagonist of KIR (e.g., lirilumab).

In some aspects, a composition disclosed herein (e.g., an antigen-HLA complex, a cell expressing a modified HLA, or a vaccine disclosed herein) is administered to a subject in combination with another anticancer agent, e.g., a chemotherapy, a cytokine, a radiation therapy, a surgery, or any combination thereof. In some aspects, the additional anticancer agent comprises a treatment selected from irradiation and/or chemotherapy, e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, or camptothecin+apo21/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al, Cancer Research 2005; 65:4799-808), c-FLIP (cellular FLICE-inhibitory protein) modulators (e.g., natural and synthetic ligands of PPARy (peroxisome proliferator-activated receptor Y), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3P inhibitors, IAP inhibitors and/or genotoxic drugs.

In some aspects, the anticancer agent comprises one or more anti-proliferative cytotoxic agents. In some aspects, the anticancer agent comprises an alkylating agents (including, without limitation, nitrogen mustards, ethylenimine derivatives, alkyl sulfonates, nitrosoureas and triazenes). In some aspects, the anticancer agent comprises uracil mustard, chlormethine, cyclophosphamide (CYTOXAN®) fosfamide, melphalan, chlorambucil, pipobroman, triethylenemelamine, triethylenethiophosphoramine, busulfan, carmustine, lomustine, streptozocin, dacarbazine, temozolomide, and any combination thereof.

In some aspects, the anticancer agent comprises an antimetabolite (including, without limitation, folic acid antagonists, pyrimidine analogs, purine analogs and adenosine deaminase inhibitors). In some aspects, the anticancer agent comprises methotrexate, 5-fluorouracil, floxuridine, cytarabine, 6-mercaptopurine, 6-thioguanine, fludarabine phosphate, pentostatine, gemcitabine, and any combination thereof.

In some aspects, the anticancer agent comprises a taxane, paclitaxel (e.g., TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone Bl, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone BIO, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5 (10)-trien-3-ol, cyclostreptin, isolaulimalide, laulimalide, 4-epi-7-dehydroxy-14, 16-didemethyl-(+)-discodermolides, and cryptothilone 1, a microtubuline stabilizing, and any combination thereof.

In some aspects, the anticancer agent comprises a lymphodepleting chemotherapy. In some aspects, the lymphodepleting chemotherapy is administered prior to the modified immune cells. In some aspects, the lymphodepleting chemotherapy comprises cyclophosphamide. In some aspects, the lymphodepleting chemotherapy comprises fludarabine. In some aspects, the lymphodepleting chemotherapy comprises cyclophosphamide and fludarabine.

In some aspects, the anticancer agent comprises a cytokine. In some aspects, the cytokine comprises an interleukin. In some aspects, the cytokine is selected from IL2, IL7, IL12, IL15, IL17, IL21, granulocyte macrophage colony-stimulating factor (GM-CSF), and interferon (IFN)-α. In some aspects, the cytokine comprises IL2.

### II.C. Methods of Engineering a Modified HLA

In some aspects of the present disclosure, the HLA-binding pocket of the HLA molecule is modified using a gene editing tool. In some aspects, the modification occurs in a cell expressing the HLA molecule ex vivo. In some aspects, the modification occurs in a cell expressing the HLA molecule in vitro. In some aspects, the modification occurs in a cell expressing the HLA molecule in vivo. In some aspects, the methods disclosed herein comprise genetically modifying an HLA molecule in a human subject using an in vivo gene editing tool.

Any gene editing tools can be used in the methods of the present disclosure. In some aspects, the gene editing tool comprises CRISPR/Cas9. In some aspects, the gene editing tool comprises a TALEN. In some aspects, the gene editing tool comprises a zinc finger nuclease. In some aspects, the gene editing tool comprises a meganuclease.

### II.C.1. CRISPR/Cas9

In some aspects, the gene editing tool that can be used in the present disclosure comprises a CRISPR/Cas system. Such systems can employ, for example, a Cas9 nuclease, which in some instances, is codon-optimized for the desired cell type in which it is to be expressed (e.g., antigen presenting cells). CRISPR/Cas systems use Cas nucleases, e.g., Cas9 nucleases, that are targeted to a genomic site by complexing with a synthetic guide RNA (gRNA) that hybridizes to a target DNA sequence immediately preceding an NGG motif recognized by the Cas nuclease, e.g., Cas9. A double-strand break three nucleotides upstream of the NGG motif is produced. Additional fusions with other enzymes can lead to site-specific base editing in the absence of a double stranded break. A unique capability of the CRISPR/Cas9 system is the ability to simultaneously target multiple distinct genomic loci by co-expressing a single Cas9 protein with two or more gRNAs (e.g., at least one, two, three, four, five, six, seven, eight, nine or ten gRNAs).

A CRISPR system used herein can use a fused crRNA-tracrRNA construct (i.e., a single transcript) that functions with the codon-optimized Cas9. This single RNA is often referred to as a guide RNA or gRNA or single guide RNA, or sgRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid.

The gRNA expression cassette and the Cas9 expression cassette are then introduced into the cell. See, for example, Mali P et al., (2013) Science 2013 Feb. 15; 339(6121):823-6; Jinek M et al., Science 2012 Aug. 17; 337(6096):816-21; Hwang W Y et al., Nat Biotechnol 2013 March; 31(3):227-9; Jiang W et al., Nat Biotechnol 2013 March; 31(3):233-9; Cronican et al., ACS Chem. Biol. 5(8):747-52 (2010); and Cong L et al., Science 2013 Feb. 15; 339(6121):819-23, each of which is herein incorporated by reference in its entirety.

In some aspects, the HLA-binding pocket of the HLA molecules is modified using CRISPR/Cas9.

### II.C.2. TALENs

In some aspects, the gene editing tool that can be used in the present disclosure comprises a nuclease agent, such as a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al., (2010) PNAS 10.1073/pnas.1013133107; Scholze & Boch (2010) Virulence 1:428-432; Christian et al., Genetics (2010) 186:757-761; Li et al., (2010) Nuc. Acids Res. (2010) doi:10.1093/nar/gkq704; and Miller et al., (2011) Nature Biotechnology 29:143-148; all of which are herein incorporated by reference in their entirety.

In some aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

### II.C.3. Zinc Finger Nucleases

In some aspects, the gene editing tool that can be used in the present disclosure comprises a nuclease agent, such as a zinc-finger nuclease (ZFN) system. Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired, one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some aspects, the DNA-binding domains of individual ZFNs comprise between three and six individual zinc finger repeats and can each recognize between 9 and 18 base pairs.

Zinc finger binding domains can be engineered to bind to a sequence of choice. See, for example, Beerli et al., (2002) Nature Biotechnol. 20:135-141; Pabo et al., (2001) Ann. Rev. Biochem. 70:313-340; Isalan et al., (2001) Nature Biotechnol. 19:656-660; Segal et al., (2001) Curr. Opin. Biotechnol. 12:632-637; Choo et al., (2000) Curr. Opin. Struct. Biol. 10:411-416; 2002-2003 Catalogue, New England Biolabs, Beverly, Mass.; and Belfort et al., (1997) Nucleic Acids Res. 25:3379-3388; each of which is herein incorporated by reference in its entirety. An engineered zinc finger binding domain can have a novel binding specificity, compared to a naturally-occurring zinc finger protein. Engineering methods include, but are not limited to, rational design and various types of selection.

Any means for target DNA sequence selection can be used in the methods described herein. A target site generally has a length of at least 9 nucleotides and, accordingly, is bound by a zinc finger binding domain comprising at least three zinc fingers. However, binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al., (1997) Nucleic Acids Res. 25:3379-3388. Additional enzymes which cleave DNA are known (e.g., 51 Nuclease; mung bean nuclease; pancreatic DNaseI; micrococcal nuclease; yeast HO endonuclease; see also Linn et al., (eds.) Nucleases, Cold Spring Harbor Laboratory Press, 1993). One or more of these enzymes (or functional fragments thereof) can be used as a source of cleavage domains.

### II.C.4. Meganucleases

In some aspects, the gene editing tool that can be used is a meganuclease system. Meganuclease domains, structure and function are known, see, for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346; each of which is herein incorporated by reference in its entirety.

### II.D. Methods of Enriching a Target Population of T Cells

Without being bound by any particular mechanism, the modified HLA molecules described herein have increased affinity for antigens. As such, the modified HLA molecules allow for surface display of antigens recognized by low affinity TCRs, allowing for the identification of novel TCRs and enrichment of T cells expressing such novel TCRs.

Some aspects of the present disclosure are further directed to methods of identifying novel T cell receptors (TCRs) that are capable of biding a target antigen-HLA complex, comprising (i) contacting a target antigen with antigen-HLA complex comprising a binding pocket that is modified to increase the affinity of the HLA to an antigen, and (ii) contacting a plurality of TCRs with the target antigen-HLA complex. Some aspects of the present disclosure are further directed to methods of identifying novel T cell receptors (TCRs) that are capable of biding a target antigen-HLA complex, comprising (i) contacting a target antigen with an engineered antigen-presenting cell, wherein the engineered antigen-presenting cell comprises a binding pocket that is modified to increase the affinity of the HLA to an antigen, and (ii) contacting a plurality of TCRs with the target antigen-HLA complex.

In some aspects, following the contacting, the enriched population of T cells comprises a higher number of T cells capable of binding the antigen-HLA complex relative to the number of T cells capable of binding the antigen-HLA complex prior to the contacting.

In some aspects, T cells that associate with the antigen-HLA complex are isolated. Using conventional methods, the TCRs that bind to the antigen-HLA complex can be identified and sequenced. The identified TCRs can then be expressed recombinantly in an immune cells, conferring upon that immune cell the ability to target the antigen.

Some aspects of the present disclosure are directed to a method of selecting a T cell capable of targeting a tumor cell. In some aspects, the method comprises contacting a population of isolated T cells in vitro with an antigen-HLA complex, wherein the HLA comprises a modified HLA-binding pocket, as described herein. In some aspects, the T cells are obtained from a human subject.

The T cells obtained from the human subject can be any T cells disclosed herein. In some aspects, the T cells obtained from the human subject are tumor infiltrating lymphocytes (TILs). In some aspects, the method further comprises administering to the human subject the enriched T cells. In some aspects, the subject is preconditioned prior to receiving the T cells, as described herein.

### III. Compositions of the Disclosure

Some aspects of the present disclosure are directed to an HLA molecule comprising a modified HLA-binding pocket. The modified HLA molecule can comprise any modified HLA molecule disclosed herein, e.g., in Section II.A., above. The modified HLA molecules described herein have increased affinity for antigens. As such, some aspects of the present disclosure are directed to an antigen-HLA complex, comprising an HLA molecule comprising a modified binding pocket, as disclosed herein.

In some aspects, the antigen is a tumor antigen. In some aspects, the antigen is an antigen expressed by a pathogen. In some aspects, the antigen is a viral antigen. In some aspects, the antigen is a bacterial antigen. In some aspects, the antigen is a fungal antigen. In some aspects, the antigen is a polypeptide that is less than about 30 amino acids, less than about 29 amino acids, less than about 28 amino acids, less than about 27 amino acids, less than about 26 amino acids, less than about 25 amino acids, less than about 24 amino acids, less than about 23 amino acids, less than about 22 amino acids, less than about 21 amino acids, less than about 20 amino acids, less than about 19 amino acids, less than about 18 amino acids, less than about 17 amino acids, less than about 16 amino acids, less than about 15 amino acids, less than about 14 amino acids, less than about 13 amino acids, less than about 12 amino acids, less than about 11 amino acids, less than about 10 amino acids in length. In some aspects, the antigen is an antigen recognized by a TCR with low affinity (e.g., low affinity antigens).

### III.A. Cells Comprising Modified HLAs

Some aspects of the present disclosure are directed to a cell comprising a modified HLA molecule disclosed herein or a nucleic acid molecule encoding the modified HLA molecule. In some aspects, the cell is a mammalian cell. In some aspects, the cell is a human cell. In some aspects, the cell is an antigen-presenting cell. In some aspects, the cell is a dendritic cell. In some aspects, the cell is an artificial antigen-presenting cell. In some aspects, the artificial antigen-presenting cell comprises a bead (e.g., a silicate bead, a glass bead, a metal bead, or a combination thereof), a nanovesicle, a microvesicle, an exosome, an endosome, or any combination thereof. In some aspects, the cell is in vivo. In some aspects, the cell is ex vivo.

In some aspects, the T cell is isolated from a human subject. In some aspects, the cell is an allogenic cell. In some aspects, the human subject is the same subject that will ultimately receive the T cell therapy. In some aspects, the cell is a donor cell, i.e., a cell obtained from a subject other than the subject that may ultimately receive the cell.

In some aspects, the cell is a cell that does not naturally express CD3, wherein the cell has been modified to express CD3. In some aspects, the cell comprises a transgene encoding CD3, wherein the transgene is expressed by the cell. In some aspects, the cell comprises a transgene encoding a protein that activates expression of endogenous CD3 by the cell. In some aspects, the cell comprises a transgene encoding a protein or siRNA that inhibits an inhibitor of CD3 expression in the cell. In some aspects, the transgene is incorporated into the genome of the cell. In some aspects, the transgene is not incorporated into the genome of the cell.

In some aspects, the cell is derived from a pluripotent stem cell, e.g., an embryonic stem cell (ESC), a hematopoietic stem cell (HSC), or an induced pluripotent stem cell (iPSC). In some aspects, the cell is isolated from peripheral blood mononuclear cells (PBMCs). In some aspects, the cell is isolated from a tumor biopsy, e.g., a tumor infiltrating lymphocyte (TIL).

### III.B. Nucleic Acid Molecules and Vectors

Certain aspects of the present disclosure are directed to nucleic acid molecules encoding a modified HLA molecule disclosed herein. Some aspects of the present disclosure are directed to vectors comprising a nucleic acid molecule encoding a modified HLA disclosed herein. In some aspects, the vector is a viral vector. In some aspects, the vector is a viral particle or a virus. In some aspects, the vector is a mammalian vector. In some aspects, the vector is a bacterial vector.

In certain aspects, the vector is a retroviral vector. In some aspects, the vector is selected from the group consisting of an adenoviral vector, a lentivirus, a Sendai virus, a baculoviral vector, an Epstein Barr viral vector, a papovaviral vector, a vaccinia viral vector, a herpes simplex viral vector, and an adeno associated virus (AAV) vector. In particular aspects, the vector is an AAV vector. In some aspects, the vector is a lentivirus. In particular aspects, the vector is an AAV vector. In some aspects, the vector is a Sendai virus. In some aspects, the vector is a hybrid vector. Examples of hybrid vectors that can be used in the present disclosure can be found in Huang and Kamihira, Biotechnol. Adv. 31(2):208-23 (2103), which is incorporated by reference herein in its entirety.

### III.C. Vaccines

Certain aspects of the present disclosure a cancer vaccine comprising an antigen-HLA complex, wherein the HLA comprises a modified HLA-binding pocket, as disclosed herein. In some aspects, the antigen is a tumor antigen. In some aspects, the vaccine further comprises one or more excipient. In some aspects, the vaccine further comprises one or more additional peptides. In some aspects, the one or more additional peptides comprise one or more additional epitopes.

All of the various aspects, aspects, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1: Methods

### Cell Samples

Peripheral blood samples were obtained from healthy donors after Institutional Review Board approval. Mononuclear cells were obtained via density gradient centrifugation (Ficoll-Paque PLUS; GE Healthcare, Chicago, IL). K562 is an erythroleukemic cell line with defective HLA expression. T2 is a T cell leukemia/B-LCL hybrid cell line. Jurkat 76 is a T cell leukemic cell line lacking TCR and CD8 expression. The K562, T2, and Jurkat 76 cell lines were grown in RPMI 1640 supplemented with 10% FBS and 50 μg/ml gentamicin (Thermo Fisher Scientific). The HEK293T cell line was grown in DMEM supplemented with 10% FBS and 50 μg/ml gentamicin. The K562, T2, and HEK293T cells were obtained from the American Type Culture Collection (ATCC; Manassas, VA). TILs isolated from a metastatic melanoma patient were grown in vitro. High-resolution HLA DNA typing was performed on the TIL sample.

### Peptides

Synthetic peptides were dissolved at 50 mg/ml in DMSO. Peptides used were A2-restricted heteroclitic NY-ESO-1₁₅₇₋₁₆₅ (SLLMWITQV), gp100₁₅₄₋₁₆₂ (KTWGQYWQV), and HIV pol₄₇₆₋₄₈₄ (ILKEPVHGV), and HLA-A24:02-restricted gp100-intron4₁₇₀₋₁₇₈ (VYFFLPDHL), gp100-intron4₁₆₁₋₁₈₀ (PSQPIIHTCVYFFLPDHLSF), gp100-intron 4₁₆₆₋₁₈₅ (IHTCVYFFLPDHLSFGRPFH), wild-type WT1₂₃₅₋₂₄₃ (CMTWNQMNL), heteroclitic WT1₂₃₅₋₂₄₃ (CYTWNQMNL), HTLV-1 tax₃₀₁₋₃₀₉ (SFHSLHLLF), and HIV env₅₈₄₋₅₉₂ (RYLRDQQLL) peptides. HIV pol₄₇₆₋₄₈₄, HTLV-1 tax₃₀₁₋₃₀₉, and HIV env₅₈₄₋₅₉₂ peptides were utilized as negative controls. Example peptide sequences tested for peptide-HLA binding assay and measurement of peptide-exchange efficiency are listed in Table 1.

### Genes

All the HLA-A*24:02 genes were linked to the truncated NGFR (ΔNGFR) gene using a Furin-SGSG-F2A sequence and cloned into the pMX retrovirus plasmid. The full-length gp100 gene was purchased from Dharmacon (Lafayette, CO). Genomic DNA of gp100 was isolated using PureLink Genomic DNA Mini Kit (Thermo Fisher Scientific, Waltham, MA). All genes were cloned into the pMX retrovirus vector and transduced using the 293GPG cell-based retrovirus system.

### Transfectants

Jurkat 76/CD8 cells were transduced with individual TCRα and TCRβ genes as reported previously (see, e.g., T. Ochi et al., Optimization of T-cell reactivity by exploiting TCR chain centricity for the purpose of safe and effective antitumor TCR gene therapy. Cancer Immunol. Res. 3, 1070-1081 (2015), which is incorporated by reference herein in its entirety). The Jurkat 76/CD8-derived TCR transfectants were purified (>95% purity) using CD3 Microbeads (Miltenyi Biotec, Bergisch Gladbach, Germany). PG13-derived retrovirus supernatants were used to transduce TCR genes into human primary T cells. T2 cells were retrovirally transduced with HLA-A*24:02 (Wild-type) or A*24:02 (A81L, L82R, R83G) to generate T2-A*24:02, or T2-A*24:02 (A81L, L82R, R83G), respectively. β2m sgRNA plasmid (Origene, Rockville, MD) was electroporated into T2 cells using Gene Pulser Xcell (Bio-Rad, Hercules, CA). The cells were stained with biotin-conjugated anti-β2m antibody (clone 2M2; BioLegend, San Diego, CA) and β2m-negative cells were isolated using anti-biotin Microbeads (Miltenyi Biotec, Bergisch Gladbach, German) to obtain β2m-knockout T2 (T2/β2m KO) cells. T2/β2m KO cells were retrovirally transduced with β2m linked HLA-A*02:01 (Wild-type or L81A) to generate T2/β2m KO/β2m-A*02:01 (Wild-type or L81A). All the HLA-A*24:02 genes were tagged with the ΔNGFR gene as described above, and the ΔNGFR+ cells were purified (>95% purity) and used in subsequent experiments.

### Flow Cytometry

Cell surface molecules were stained with a PC5-conjugated anti-CD8 mAb (clone B9.11; Beckman Coulter, Brea, CA), FITC-conjugated anti-NGFR (clone ME20.4; BioLegend, San Diego, CA), and APC/Cy7-conjugated anti-CD3 (clone UCHT1; BioLegend, San Diego, CA). Dead cells were discriminated with the LIVE/DEAD Fixable Aqua Dead Cell Stain Kit (Life Technologies, Carlsbad, CA). For intracellular staining, cells were fixed and permeabilized by using a Cytofix/Cytoperm kit (BD Biosciences Franklin Lakes, NJ). Stained cells were analyzed with flow cytometry (BD Biosciences Franklin Lakes, NJ) and data analysis was performed using FlowJo (BD Franklin Lakes, NJ).

### Peptide-HLA Binding Assay

T2-A24 (Wild-type) cells, or T2-A24 cells with a single amino acid substitution at position 81, 82, or 83 were pulsed with 50 μg/ml of biotinylated peptide overnight at 37° C. After intensive washing, the cells were stained by PE-conjugated streptavidin (SA-PE), washed and fluorescence intensity was measured by flow cytometry analysis.

### Cytokine ELISPOT Analysis

IL-2 and IFN-γ ELISPOT analysis was conducted as described previously. For the IL-2 ELISPOT assay, PVDF plates (Millipore, Bedford, MA) were coated with capture mAb (SEL002; R&D Systems, Minneapolis, MN). T cells were incubated with 2×10⁴ target cells per well in the presence or absence of peptide for 20-24 hours at 37° C. for 20-24 hours at 37° C. The plates were washed and incubated with biotin-conjugated detection mAb (SEL002; R&D Systems, Brandywine, MD). After washing, alkaline phosphatase-conjugated streptavidin (Jackson ImmunoResearch, West Grove, PA) was added. The plates were washed and incubated with NBT/BCIP (nitroblue tetrazolium/5-bromo-4-chloro-3-indolyl phosphate; Promega, Madison, WI), and IL-2 spots were developed. For IFN-γ ELISPOT analysis, PVDF plates (Millipore, Bedford, MA) were coated with the capture mAb (1-DIK; MABTECH, Mariemont, OH), and T cells were incubated with 2×10⁴ target cells per well for 20-24 hours at 37° C. The plates were subsequently washed and incubated with a biotin-conjugated detection mAb (7-B6-1; MABTECH). HRP-conjugated SA (Jackson ImmunoResearch, West Grove, PA) was then added and IFN-γ spots were developed. The reaction was stopped by rinsing thoroughly with cold tap water. ELISPOT plates were scanned and counted using an ImmunoSpot plate reader and ImmunoSpot version 5.0 software (Cellular Technology Limited, Shaker Heights, OH).

### Expansion of CD8⁺ TILs in an HLA-A24-Restricted Peptide-Specific Manner

CD8⁺ TILs were purified through negative magnetic selection using a CD8⁺ T Cell Isolation Kit (Miltenyi Biotec, Bergisch Gladbach, Germany). HLA-A*24:02 aAPCs were pulsed with 10 μg/ml class I-restricted peptides of interest for 6 hours. The aAPCs were then irradiated at 200 Gy, washed, and added to the TILs at an effector to target (E:T) ratio of 20:1. After forty-eight hours, 10 IU/ml IL-2 (Novartis, Basel, Switzerland), 10 ng/ml IL-15 (Peprotech, East Windsor, NJ), and 30 ng/ml IL-21 (Peprotech, East Windsor, NJ) were added to the cultures every three days.

Expansion of Primary CD8⁺ T Cells Transduced with TCRs

CD3⁺ T cells were purified through negative magnetic selection using a Pan T Cell Isolation Kit (Miltenyi Biotec, Bergisch Gladbach, Germany). Purified T cells were stimulated with aAPC/mOKT3 irradiated with 200 Gy at an ET ratio of 20:1. After overnight incubation, activated T cells were retrovirally transduced with cloned TCR genes via centrifugation for 1 hour at 1,000 g at 32° C. for three consecutive days. After forty-eight hours, 100 IU/ml IL-2 and 10 ng/ml IL-15 were added to the TCR-transduced T cells. The culture medium was replenished every 2-3 days.

Production of Human Cell-Based pHLA Multimers

The affinity-matured HLA-A*24:02 gene was engineered to carry a Glu (E) residue in lieu of the Gln (Q) residue at position 115 of the α2 domain and a mouse Kb gene-derived α3 domain instead of the HLA class I α3 domain. The soluble A*24:02^{Q115E}-K^{b} gene was generated by fusing the extracellular domain of the affinity-matured HLA-A*24:02 gene with a Gly-Ser (GS) flexible linker followed by a 6× His tag. HEK293T cells were individually transduced with the soluble A*24:02^{Q115E}-K^{b} gene using the 293GPG cell-based retrovirus system. Stable HEK293T cells expressing soluble affinity-matured A*24:02^{Q115E}-K^{b} gene were grown until confluent and the medium was changed. Forty-eight hours later, conditioned medium was harvested and used immediately or frozen at −80° C. for later use. The soluble A*24:02^{Q115E}-K^{b}-containing supernatant produced by HEK293T transfectants was mixed with 100 μg/ml of A*24:02-restricted peptide of interest and incubated overnight at 37° C. for in vitro peptide exchange. Soluble monomeric A*24:02^{Q115E}-K^{b} loaded with peptide was dimerized using an anti-His mAb (clone AD1.1.10; Abcam) conjugated to a fluorochrome such as phycoerythrin (PE) at a 2:1 molar ratio for 2 hours at room temperature or overnight at 4° C. The concentration of functional soluble A*24:02^{Q115E}-K^{b} molecules was measured by specific ELISA using an anti-HLA class I mAb (clone W6/32) and an anti-His tag biotinylated mAb (clone AD1.1.10, R&D systems) as capture and detection Abs, respectively.

pHLA Multimer Staining

T cells (2×10⁵) were incubated for 30 minutes at 37° C. in the presence of 50 nM dasatinib (LC Laboratories, Woburn, MA). The cells were then washed and incubated with 5-10 μg/ml of pHLA multimer for 30 minutes at room temperature, and R-phycoerythrin-conjugated AffiniPure Fab fragment goat anti-mouse IgG1 (Jackson ImmunoResearch, West Grove, PA) was added for 15 minutes at 4° C. Next, the cells were washed three times and co-stained with an anti-CD8 mAb for 15 minutes at 4° C. Dead cells were discriminated using the LIVE/DEAD Fixable Dead Cell Stain Kit.

### Measurement of Peptide Exchange Efficiency Using ELISA

Efficiency of peptide-exchange in monomer was assessed using a competition binding assay and enzyme-linked immunosorbent assay (ELISA). Monomer was loaded with 100 μg/ml biotinylated peptide with sequence ₃₇TYFSLNNK(-biotin)F₄₅ derived from Adenovirus 5 Hexon and incubated overnight at 37° C. Biotinylated peptide-HLA was purified and exchanged into Phosphate Buffered Saline (PBS) using Amicon Ultra filters (molecular weight cut-off (MWCO) 10 kDa) (MilliporeSigma, Burlington, MA), and mixed with 1 mg/ml peptide of interest and incubated overnight at 37° C. ELISA plates were coated with anti-HLA-class I mAb (clone W6/32) at 10 μg/mL in PBS overnight at 4° C. The plates were washed and blocked with 10% nonfat dry milk in PBS for 30 minutes at room temperature. pHLA monomer was added and incubated for 2 hours at room temperature. After washing, the plates were incubated with streptavidin-conjugated alkaline phosphatase for 30 minutes at room temperature. Finally, the plates were washed and incubated with p-nitrophenyl phosphate (PNPP) substrate (Pierce, Rockford, IL) at room temperature. The reaction was terminated by adding 1 mol/L NaOH. The optic density (OD) (405 nm) was read (Spectramax 190 Microplate Reader; Molecular Devices, Sunnyvale, CA). The OD values from the control wells containing nonbiotinylated peptide were subtracted from the OD values in test wells containing biotinylated peptide. The efficiency of peptide exchange for each monomer was calculated as follows: Peptide exchange efficiency=[1−(OD value with peptide/OD value with DMSO)]×100. Every sample was assayed in triplicate wells.

### Statistical Analysis

Statistical analysis was performed using GraphPad Prism (GraphPad Software, San Diego, CA). To determine whether two groups were significantly different for a given variable, an analysis using Welch's t-test (two-sided) was conducted. P values <0.05 were considered significant.

### Example 2: Leucine Substitution at Position 81 (A81L) in HLA-A*24:02

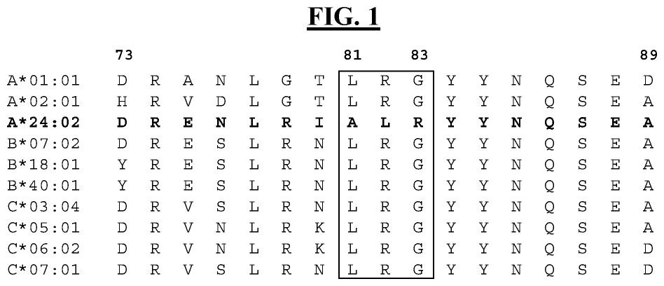

During assessments of peptide immunogenicity, allelic variations between HLA class I alleles were investigated. Amongst key residues forming the HLA class I peptide binding pockets, the position 81 within the F pocket showed two polymorphisms: HLA-A and -B alleles expressed either an alanine (Ala) or a leucine (Leu) residue, while HLA-C alleles only expressed a Leu residue. Moreover, with the exception of most members of the A24-supertype, Leu was the residue most frequently found in position 81 amongst all HLA class I alleles. Further, as shown in FIG. 1 (FIG. 1) when aligning only the most prevalent HLA-class I alleles found within the general population, the α1 domain of HLA-A*24:02 differed from those of other HLA alleles at positions 81 to 83, suggesting that these residues may uniquely define HLA-A*24:02 and influence its peptide binding.

While the A24-supertype is represented in all ethnic groups and represents the second most frequent HLA-A allele in the world, it is the most prevalent in Asian ethnicities, with HLA-A*24:02 being the most prevalent allele amongst the Japanese population. Thus, in attempts to understand how HLA-A*24:02 presents antigens, the biological effects of amino acid substitutions within the HLA-A*24:02 peptide binding pocket were investigated.

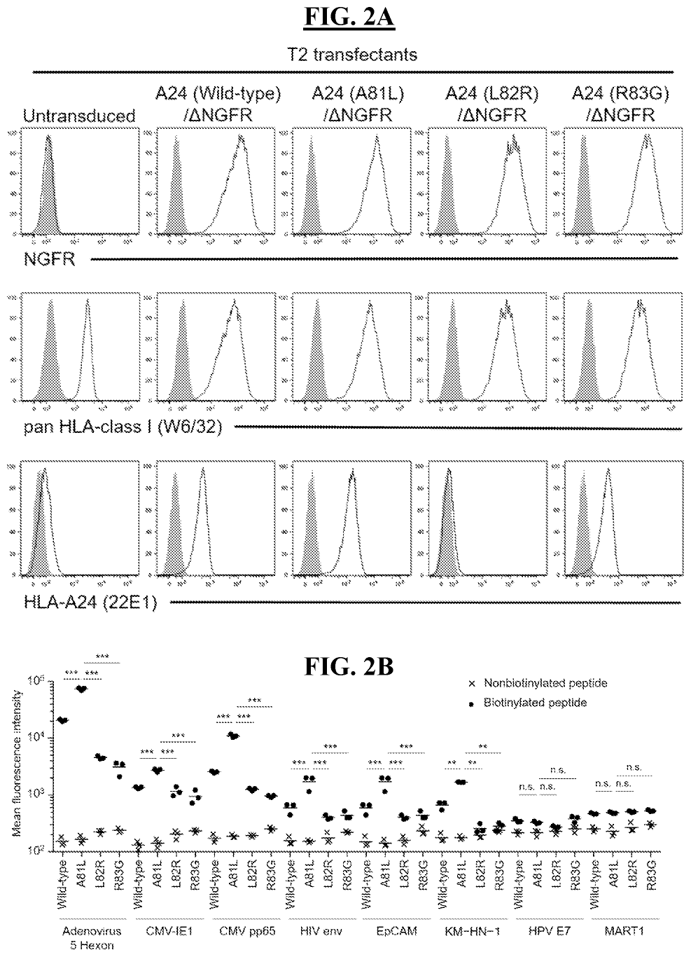

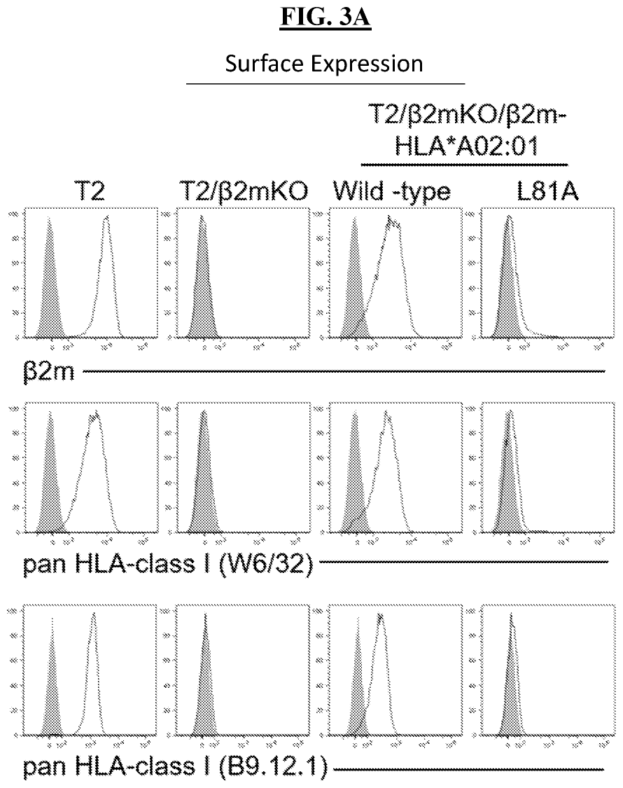

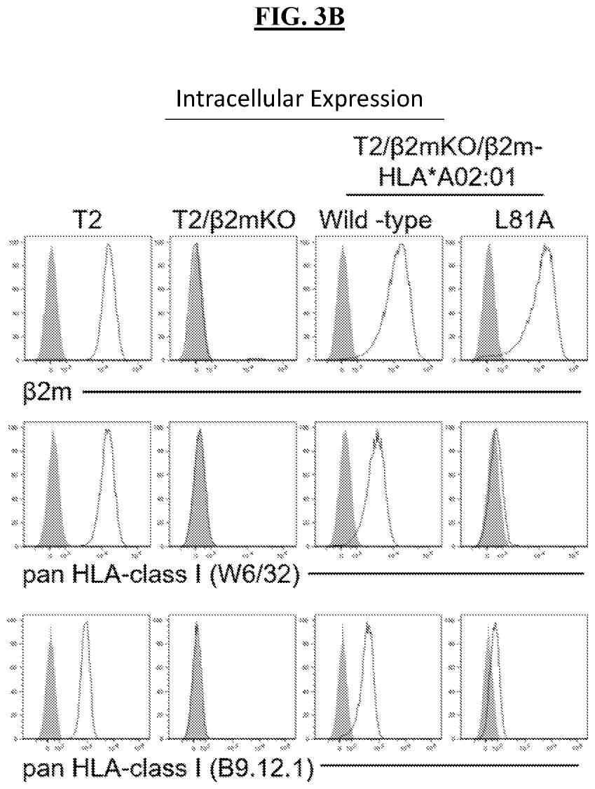

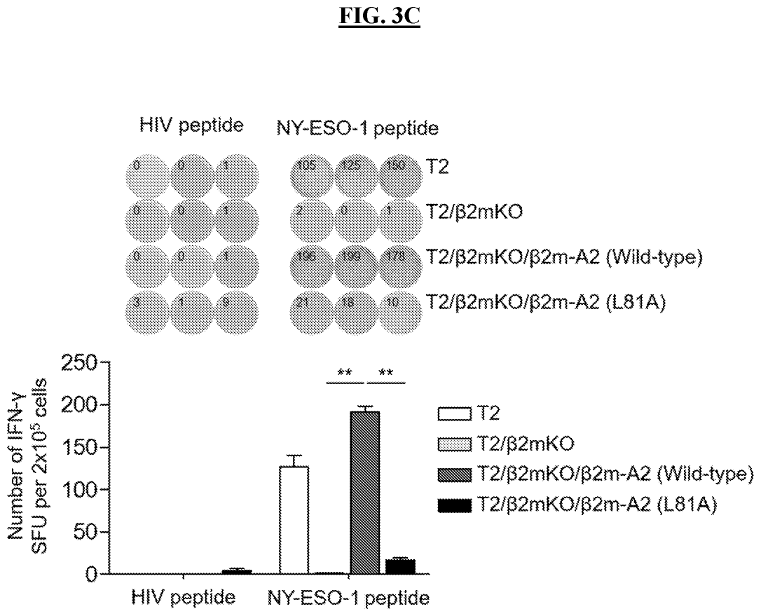

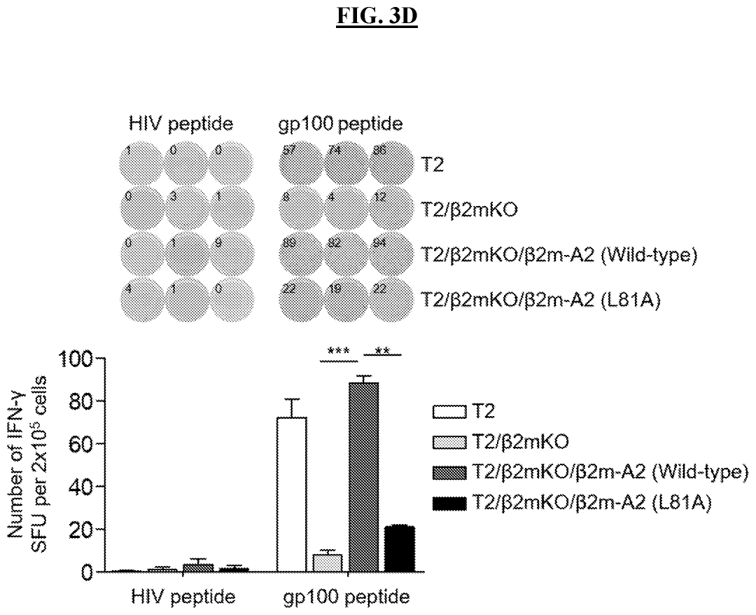

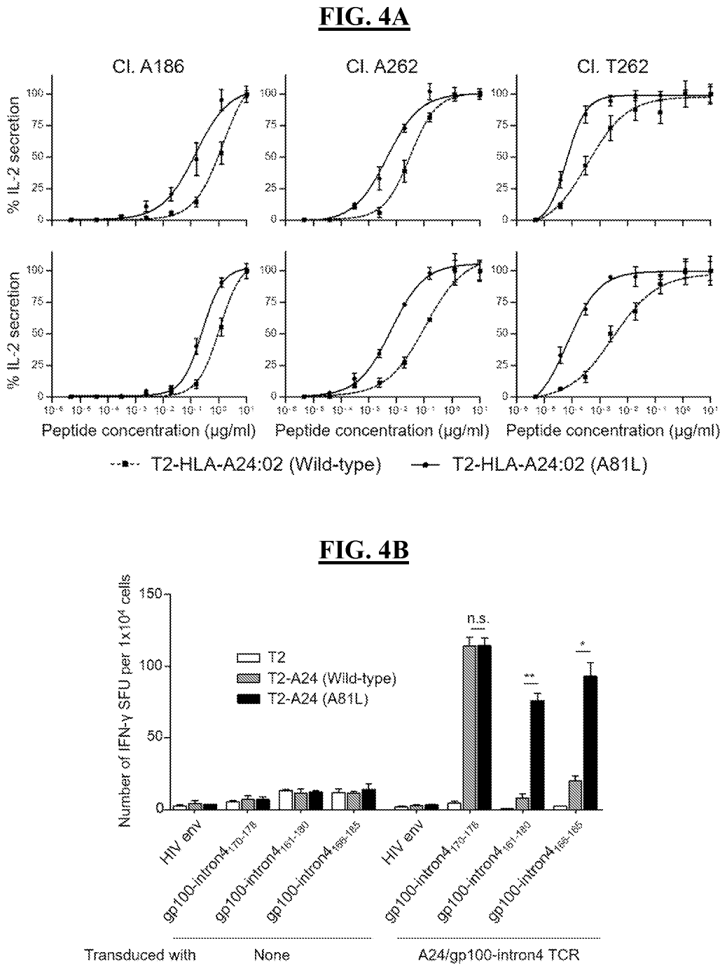

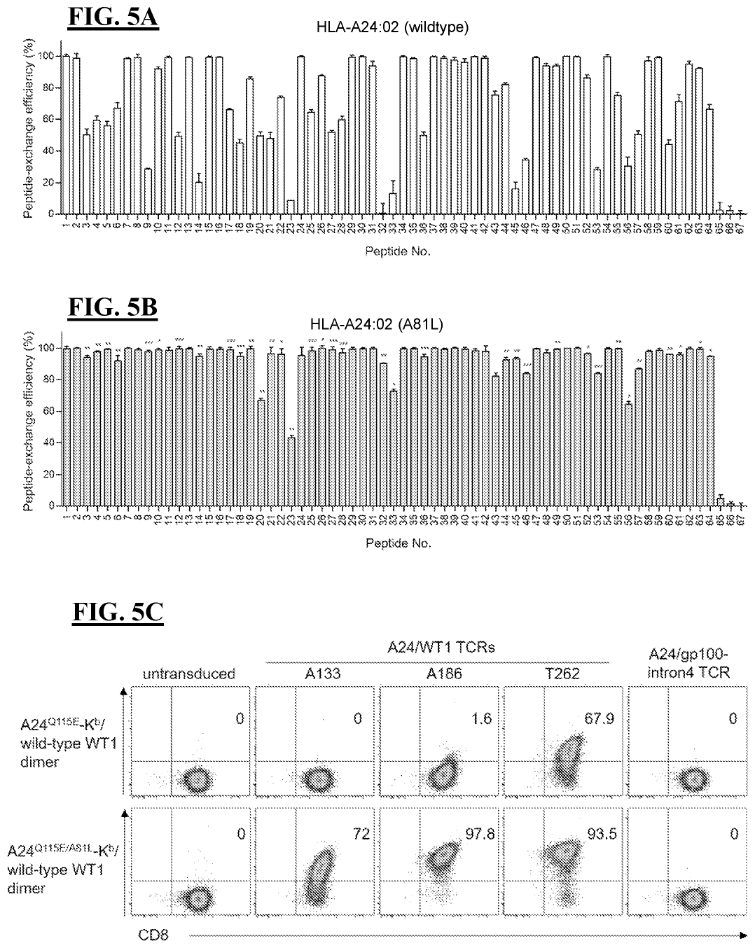

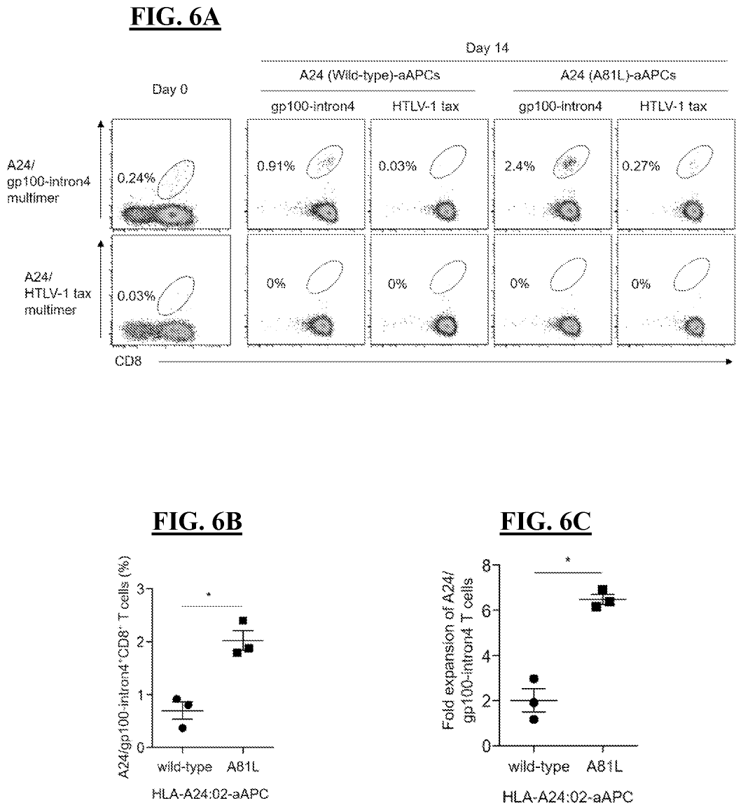

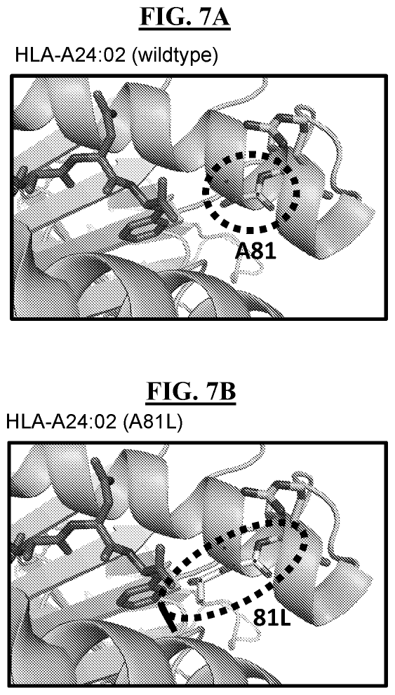

Using T2 cells, HLA-A*24:02 constructs expressing single amino acid substitutions at positions 81-83 were transduced, substituting each position with the most common residues found in other HLA class I alleles (FIG. 1). T2 lines expressing HLA-A*24:02 with an Ala to Leu substitution at position 81 (A81L), Leu to arginine (Arg) at position 82 (L82R), or Arg to Glycine at position 83 (R83G) were all successfully transduced. However, stark differences in anti-HLA antibody binding were observed (FIG. 2A). Whereas the pan-anti-HLA-class I antibody (clone W6/32) was able to similarly bind all HLA-A*24:02-transduced T2 cells with greater mean florescence intensity (MFI) than untransduced T2 cells, we found that anti-HLA-A*24 (clone 22E1) lost the ability to detect the mutant HLA-A*24:02 (L82R).

Whether T2 transfectants expressing HLA-A*24:02 constructs were capable of presenting antigens was investigated in order to generate a super-agonist HLA, allowing for one engineered HLA molecule to accommodate many natural peptides, as opposed to manufacturing many heteroclitic peptides. T2 cells were pulsed with an array of known HLA-A*24:02-restricted peptides derived from viral and tumor-associated antigens and cell surface presentation of pHLA complexes were analysis by flow cytometry. Consistent with increased pan-HLA-class I expression observed in T2 transfectants, it was found that all HLA-A*24:02 constructs, including the HLA-A*24:02 (L82R) construct, were capable of presenting peptides (FIG. 2B), suggesting that substitutions at position 81-83 did not alter surface expression or folding of HLA-A*24:02 but rather affected epitope recognition by anti-HLA-A*24 (clone 22E1) antibody. However, assays found that T2-HLA-A*24:02 (A81L) had a significantly greater ability to present HLA-A*24:02-restricted peptides compared to wild-type or other HLA-A*24:02 mutants. These results highlight the importance of this substitution in peptide anchoring and suggest that the HLA-A*24:02 (A81L) super-agonist construct can better sustain the presentation of HLA-A*24:02 antigens on the cell surface of antigen-presenting cells (APCs).

### Example 3: Surface Expression of HLA-A*02:01

Expressing a Leu residue at position 81 renders HLA-A*02:01 more similar to other common HLA-B and C alleles than to HLA-A*24:02 (FIG. 1). Given that the A81L substitution in HLA-A*24:02 has a profound effect on the surface expression of pMHC complexes, whether such a substitution would alter antigen presentation involving HLA-A*02:01 was examined. To control for endogenous expression of HLA-A*02:01 in T2 cells, a β2m-linked HLA-A*02:01 (β2m-HLA-A*02:01) with an L81A substitution expressed as single chain was generated and transduced into β2m knockout T2 cells (T2/β2mKO). Consistent with previous observations, endogenous HLA expression could be detected on the surface of wild-type T2 cells, but was absent in T2/β2mKO as detected using anti-β2m or pan-anti-HLA antibodies (FIG. 3A). Unlike β2m-HLA-A*02:01 (wild-type), β2m-HLA-A*02:01 containing the L81A substitution resulted in significantly reduced expression, staining only slightly above that of T2/β2mKO (FIG. 3A). To determine if the L81A substitution only disrupted surface expression of β2m-HLA-A*02:01, we stained fix and permeabilized T2 cells and found that although intracellular β2m expression remained intact, intracellular staining using conformation dependent pan HLA class I antibodies W6/32 or B9.12.1 did not detect β2m-HLA-A*02:01 (L81A) at any greater degree than that of surface staining (FIG. 3B).

To confirm that the reduced surface expression of β2m-HLA-A*02:01 (L81A) translated to biological activity, we performed functional assays using J76/CD8 cells expressing HLA-A*02:01-restricted NY-ESO-1₁₅₇₋₁₆₅-specific TCR (clone 1G4LY) or gp100₁₅₄₋₁₆₂-specific TCR (clone gp100154). Whereas cognate peptide-pulsed T2-β2m-HLA-A*02:01 (wild-type) were able to induce IFNγ from TCR-J76/CD8 cells in ELISPOT assays (FIG. 3C), co-culture with T2-β2m-HLA-A*02:01 (L81A) resulted in significantly reduced IFNγ production (FIG. 3C). These data suggest that the reduced surface expression of β2m-HLA-A*02:01 (L81A) was not due to altered epitope recognition by pan-anti-HLA antibody, but rather, was likely due to the L81A substitution disrupting the formation of stable HLA-A*02:01 pMHC complexes.

### Example 4: HLA-A*24:02 (A81L) Heightens Peptide-Specific T Cell Responses

Whether the increased surface expression of the super-agonist HLA led to increased T cell activation was investigated. Using Jurkat 76/CD8 cells transduced with TCRs of varying avidities towards the HLA-A*24:02/WT1₂₃₅₋₂₄₃ epitope, it was found that T2-HLA-A*24:02 (A81L) displayed a greater ability to induce T cell secretion of IL-2, as compared to T2-HLA-A*24:02 (wild-type) in both conditions where WT1₂₃₅₋₂₄₃ were continuously present within culture media (FIG. 4A, top row) or pulsed (FIGS. 4A, bottom row) onto cells prior to T cell assays. Observed for all WT1₂₃₅₋₂₄₃-specific TCRs irrespective of their avidity towards pMHC, these results suggest that the A81L substitution may enhance the ability of HLA-A*24:02 to retain surface expression. As short peptides have the ability to bind HLA on the cell surface thereby bypassing potential endosomal processing, whether A81L substitution altered the ability to cross-present internalized HLA-A*24:02-restricted peptides was tested. Using T2 transfectants, 20-mer-long peptides containing HLA-A*24:02-restricted gp100int4₁₇₀₋₁₇₈ epitopes were pulsed. Consistent with its ability to enhance T cell activation towards short peptides, TCR-transduced T cells activated with long-peptide pulsed T2-HLA-A*24:02 (A81L) resulted in a greater than 4-fold increase in the number of IFNγ secreting spots as compared to HLA-A*24:02 (wild-type) (FIG. 4B).

### Example 5: HLA-A*24:02 (A81L) Multimers Robustly Stain Low-Affinity Antigen-Specific TCRs

A major limitation of TCR-based cancer immunotherapies is the ability to identify TCRs with low affinity antigens. Designing multimers that can reliably stain such TCRs can lead to more promising cancer treatment options. Having found the A81L substitution enhanced antigen presentation of naturally occurring HLA-A*24:02 peptides to T cells, whether the super-agonist HLA construct could be used in reagents to improve the study of HLA-A*24:02-restricted T cells was tested. Novel peptide exchangeable affinity-matured HLA class I multimers that were used to detect, sort and clone tumor antigen-specific TCRs from tumor infiltrates were designed previously. Using this platform, affinity-matured HLA-A*24:02 multimers expressing the A81L substitution to determine whether this substitution could further enhance the utility of these multimers in detecting low-affinity TCRs were generated. Peptide exchange efficiency with HLA-A*24:02-restricted peptides derived from an array of viral and tumor-associated antigens were tested using a cell-free assay. When compared to wild-type HLA-A*24:02 monomers, the A81L substitution significantly enhanced the efficiency of peptide exchange for all peptides having low exchange-efficiency in wild-type HLA-A*24:02 affinity-matured monomers, increasing exchange efficiency by at least 2-fold or more (FIGS. 5A-5B). Notably, for the TSPAN10₇₆₋₈₄ peptide, the affinity-matured HLA-A*24:02 (A81L) monomer resulted in detectable peptide exchange, which was not the case with the wild-type monomer (FIGS. 5A-5B, peptide 32). The A81L substitution did not compromise the specificity of peptides, as HLA-B*18-restricted peptide MAGE-A3₁₆₇₋₁₇₆, HLA-B*27-restricted peptide VEGF (UTR) and HLA-C*06-restricted peptide GAGE1/2/8₉₋₁₆ did not led to any appreciable exchange into affinity-matured HLA-A*24:02 (A81L) monomer (FIGS. 5A-5B, peptides 65-67). Having found HLA-A*24:02 (A81L) monomer capable of more efficient peptide exchange, Jurkat 76/CD8 cells expressing cognate TCRs of varying avidities with wild-type and A81L HLA-A*24:02 WT1₂₃₅₋₂₄₃ multimers were stained. Consistent with their exchange efficiency, the HLA-A*24:02 (A81L) super-agonist multimers showed a greater ability to stain low avidity TCRs as compared with HLA-A*24:02 wild-type multimers. For TCRs A133 and A186, HLA-A*24:02 (A81L) multimers resulted in staining of T cells which otherwise would not have been detected under the experimental conditions used (FIG. 5C).

### Example 6: HLA-A*24:02 (A81L) aAPC Enhances the Expansion of Tumor Antigen-Specific T Cells

In addition to enhancing detection of low avidity T cells, whether the A81L substitution would be useful in the expansion of T cells from tumor infiltrates was tested. Using the previously developed artificial antigen-presenting cells (aAPC) platform, a short-term expansion of melanoma patient-derived tumor-infiltrating lymphocytes (TIL) using aAPCs expressing HLA-A*24:02 (A81L) or wild-type, pulsed with gp100int4₁₇₀₋₁₇₈ or control HTLV-1 tax₃₀₁₋₃₀₉ peptide was performed. After two weeks of co-culture, aAPC-HLA-A*24:02 (A81L) pulsed with gp100int4₁₇₀₋₁₇₈ resulted in a greater than 2-fold expansion of antigen-specific CD8⁺ T cells as compared to aAPC expressing wild-type HLA-A*24:02 (FIG. 6A). This ability to enhance expansion of antigen specific T cells was consistent across three replicates (FIG. 6B) and resulted in a ˜6-fold expansion from baseline TILs (FIG. 6C).

The data presented herein show that the generation of a super-agonist HLA construct from HLA-A*24:02 expressing an A81L substitution heightened the activation of T cells in co-culture assays, resulting in increased cytokine secretion and T cell expansion and did not interfere with the processing and presentation of HLA-A*24:02-restricted antigens. In cell-free assays, the super-agonist HLA significantly increased the efficiency of peptide exchange towards HLA-A*24:02-restricted peptides and importantly, did not alter the restriction of peptides to HLA-A*24:02, as no increase in peptide presentation by T2 cells or in cell-free peptide-exchange assays were observed towards non-HLA-A*24:02 restricted peptides.

Given the importance of residue 81 in forming part of the F binding pocket, it was surprising that the A81L substitution did not adversely alter the binding of HLA-A*24:02-restricted peptides, but in fact led to increased surface availability of peptide-HLA-A*24:02 complexes and heightened T cell responses. This was particularly true for HLA-A*24:02-restricted peptides that poorly bind wild-type HLA-A*24:02. Like other peptide-HLA I complexes, the overall structure of HLA-A*24:02 is similar to other HLA I alleles, adopting the well-described α1 and α2 domains formed by an antiparallel β sheet and two long α helices that make-up the peptide binding interface presented to TCRs. However, unique to HLA-A*24:02 is the unusually deep B- and F-peptide binding pockets that can accommodate bulky aromatic and large hydrophobic side chains, such as anchor residues Y or F (at position 2) and F, L, I, or W (at the C-termini) of peptide ligands. Whether the A81L substitution (see FIGS. 7A-7B for in silico modeling) alters this preference is not clear; however, no bias towards the presentation of particular HLA-A*24:02-restricted peptides was observed, but an ability to enhance peptide presentation and T cell activation of all tested peptides. This observation likely places emphasis on the importance of secondary peptide anchors known to strongly influence the binding capacity of peptide ligands towards HLA-A*24:02. Indeed, the A81L substitution may affect how secondary peptide anchors influence the unique conformation that peptides adopt within HLA-A*24:02. Several crystal structures have shown that peptide ligands within HLA-A*24:02 adopt a moderately large “A” or “M” shape (in which position 5 provides an anchor) at central residues relative to other HLA I alleles, providing a unique peptide “bulge” for TCR docking and T cell recognition. Interestingly, as with the unique effects secondary anchors have on different HLA I alleles, Leu modification at position 81 may be unique to HLA-A*24:02, as substitution of position 81 within HLA-A*02:01 rendered this allele incapable of activating T cells, highlighting the importance of this residue in surface expression of pHLA complexes.

As the affinity between peptide/HLA, and thus surface expression is an important factor in determining the immunogenicity of pMHC complexes, the super-agonist modification likely increases the binding affinity of peptides to HLA-A*24:02 or modifies the conformation of peptide binding, resulting in heightened T cell activation. When applying this substitution for HLA-multimer staining, TCRs deemed low-affinity, which were not detected by wild-type monomers, could be detected using super-agonist multimers. In line with this observation, aAPC expressing HLA-A*24:02 (A81L) resulted in increased expansion of antigen-specific T cells from TILs. The ability of super-agonist multimers to detect low-affinity TCRs coupled with the ability of aAPCs to expand antigen-specific T cells might allow for a more robust scrutiny of both high and low-affinity T cell repertoires against any given epitope derived from tumor antigens.

In all, these data present a novel method to increase pHLA immunogenicity and show that modifications to the HLA binding pocket can be used to heighten T cell activation and expansion towards a broad breadth of peptides while retaining antigen restriction. The specific A81L substitution could be directly applied to HLA class I alleles which contain Ala at position 81. Namely, the majority of members of the A24-supertype (HLA-A*23 and HLA-A*24 alleles) as well as various members of the A01-(HLA-A*25, HLA-A*31, HLA-A*32 alleles), B07-(HLA-B*51 allele), B44-(HLA-B*44 allele) and B58-(HLA-B*58 allele) supertypes as well as alleles that share F pocket peptide specificity with HLA-A*24:02, but do not fit within a particular supertype (HLA-B*13, HLA-B*=38, HLA-B*49, HLA-B*52, HLA-B*53, HLA-B*57) (2). For other alleles, crystal structure guided modifications beyond position 81 could be applied and thereby alleviate the encumbrance of individually developing heteroclitic peptides for each peptide epitope.

1. A method of conditioning a subject in need of a therapy comprising modifying a human leukocyte antigen (HLA)-binding pocket of an HLA molecule expressed on a cell of the subject.

2-7. (canceled)

8. A method of increasing an immune response to an antigen in a subject in need thereof, comprising modifying an HLA-binding pocket of an HLA molecule in a cell of the subject, wherein the HLA molecule is capable of binding the antigen.

9. The method of claim 8, wherein the modifying increases a binding affinity of the HLA-binding pocket to an antigen.

10. The method of claim 8, wherein the cell is an antigen-presenting cell.

11. The method of claim 10, wherein the antigen-presenting cell is a dendritic cell.

12. The method of claim 8, wherein the HLA molecule is an HLA class I allele, optionally wherein the HLA class I allele comprises HLA-A, HLA-B, HLA-C, HLA-E, HLA-F, HLA-G, HLA-K, HLA-L, or any combination thereof.

13. (canceled)

14. The method of claim 8, wherein the HLA molecule comprises:
(a) an HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, HLA-A*34, HLA-A*36, HLA-A*43, HLA-A*66, HLA-A*68, HLA-A*69, HLA-A*74, or HLA-A*80;
(b) an HLA-B*07, HLA-B*08, HLA-B*13, HLA-B*14, HLA-B*15, HLA-B*18, HLA-B*27, HLA-B*35, HLA-B*37, HLA-B*38, HLA-B*39, HLA-B*40, HLA-B*41, HLA-B*42, HLA-B*44, HLA-B*45, HLA-B*46, HLA-B*47, HLA-B*48, HLA-B*49, HLA-B*50, HLA-B*51, HLA-B*52, HLA-B*53, HLA-B*54, HLA-B*55, HLA-B*56, HLA-B*57, HLA-B*58, HLA-B*59, HLA-B*67, HLA-B*73, HLA-B*78, HLA-B*79, HLA-B*81, HLA-B*82, or HLA-B*83;
(c) an HLA-C*05:01, HLA-C*05:03, HLA-C*05:04, HLA-C*05:05, or HLA-C*05:06; or
(d) any combination thereof.

15-18. (canceled)

19. The method of claim 8, wherein the HLA-binding pocket is A pocket, B pocket, C pocket, D pocket, E pocket, F pocket, or any combination thereof.

20-21. (canceled)

22. The method of claim 8, wherein the modifying:
a. increases the display of the antigen on the surface of the cell;
b. increases an antigen-specific T cell response;
c. increases expansion of tumor-antigen specific T cells; or
d. any combination of (a)-(c).

23-25. (canceled)

26. The method of claim 8, wherein the modifying comprises: (i) mutating an amino acid in the HLA-binding pocket of the HLA molecule; or (ii) an amino acid substitution, wherein the amino acid to be replaced is an alanine.

27-30. (canceled)

31. The method of claim 8, wherein the modifying comprises mutating an amino acid residue selected from amino acid residue 79, amino acid residue 80, amino acid residue 81, amino acid residue 82, or amino acid residue 83, wherein the positions correspond to the amino acid sequence set forth in SEQ ID NO: 1.

32-33. (canceled)

34. The method of claim 8, wherein the modifying comprises an A81L substitution, corresponding to the amino acid sequence set forth in SEQ ID NO: 1.

35. The method of claim 8, wherein the modifying is performed by a gene editing tool.

36-48. (canceled)

49. The method of claim 8, wherein the subject is afflicted with a cancer.

50. The method of claim 49, wherein the cancer is selected from the group consisting of melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers.

51. An HLA molecule comprising a modified HLA-binding pocket.

52-68. (canceled)

69. An nucleic acid or a set of nucleic acids encoding the HLA molecule of claim 51.

70-71. (canceled)

72. An HLA-antigen complex comprising the HLA molecule of claim 51 and an antigen.

73-76. (canceled)

77. A pharmaceutical composition comprising the HLA molecule of claim 51 and a pharmaceutically acceptable excipient.

78-89. (canceled)

90. A method of enriching a population of T cells obtained from a human subject comprising contacting the T cells with the HLA molecule of claim 51.


SEQUENCE LISTING (1 sequence)

SEQ ID NO: 1 - protein, 341 aa, synthetic construct
  source 1..341: note = HLA-A*24:02 amino acid sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV